(19) United States
(12) Patent Application Publication
(10) Pub. No.: US 2024/0218346 A1
(43) Pub. Date: Jul. 4, 2024

(54) PHOSPHOKETOLASE VARIANTS AND METHODS OF USE

(71) Applicant: Genomatica, Inc., San Diego, CA (US)

(72) Inventors: Hui ZHOU, Newton, MA (US); Nathan SCHMIDT, Roslindale, MA (US); Pichet PRAVESCHOTINUNT, Cambridge, MA (US); Brian CARVALHO, Somerville, MA (US)

(21) Appl. No.: 18/554,448

(22) PCT Filed: Apr. 8, 2022
(86) PCT No.: PCT/US2022/024042

§ 371 Date: Oct. 6, 2023

Related U.S. Application Data
(60) Provisional application No. 63/173,217, filed on Apr. 9, 2021.

(51) Int. Cl. C12N 9/88; C12N 15/63; C12P 1/00

(52) U.S. Cl. CPC C12N 9/88; C12N 15/63; C12P 1/00; C12Y 401/02009

(57) ABSTRACT

The disclosure provides polypeptides and encoding nucleic acids of engineered phosphoketolases. The disclosure also provides cells expressing an engineered phosphoketolase. The disclosure further provides methods for producing a bioderived compound comprising culturing cells expressing an engineered phosphoketolase.

### CROSS-REFERENCE TO RELATED APPLICATIONS

This application claims the benefit of U.S. Provisional Application No. 63/173,217, filed Apr. 9, 2021, the entire contents of which are incorporated by reference herein.

### SEQUENCE LISTING

The instant application contains a Sequence Listing which has been submitted in ASCII format via EFS-Web and is hereby incorporated by reference in its entirety. Said ASCII copy, created on Apr. 7, 2021, is named 199683-228509_PCT_SL.txt and is 39,582 bytes in size.

### FIELD OF DISCLOSURE

The present disclosure relates generally to phosphoketolase variants and methods of using such variants, and more specifically to phosphoketolase variants encoded by recombinant nucleic acids that have been introduced to a non-naturally occurring microbial organism for enhancing production of acetyl-phosphate, acetyl-CoA or a bioderived compound generated from acetyl-CoA.

### BACKGROUND DESCRIBED HEREIN

1,3-butanediol (1,3-BDO) is a four carbon diol traditionally produced from acetylene via its hydration. The resulting acetaldehyde is then converted to 3-hydroxybutyraldehdye which is subsequently reduced to form 1,3-BDO. More recently, acetylene has been replaced by the less expensive ethylene as a source of acetaldehyde. 1,3-BDO is commonly used as an organic solvent for food flavoring agents. It is also used as a co-monomer for polyurethane and polyester resins and is widely employed as a hypoglycemic agent. Optically active 1,3-BDO is a useful starting material for the synthesis of biologically active compounds and liquid crystals. Another use of 1,3-butanediol is that its dehydration affords 1,3-butadiene (Ichikawa et al. Journal of Molecular Catalysis A-Chemical 256:106-112 (2006); Ichikawa et al. Journal of Molecular Catalysis A-Chemical 231:181-189 (2005), which is useful in the manufacture synthetic rubbers (e.g., tires), latex, and resins. The reliance on petroleum based feedstocks for either acetylene or ethylene warrants the development of a renewable feedstock based route to 1,3-butanediol and to butadiene.

1,4-butanediol (1,4-BDO) is a valuable chemical for the production of high performance polymers, solvents, and fine chemicals. It is the basis for producing other high value chemicals such as tetrahydrofuran (THF) and gamma-butyrolactone (GBL). The value chain is comprised of three main segments including: (1) polymers, (2) THF derivatives, and (3) GBL derivatives. In the case of polymers, 1,4-BDO is a comonomer for polybutylene terephthalate (PBT) production. PBT is a medium performance engineering thermoplastic used in automotive, electrical, water systems, and small appliance applications. Conversion to THF, and subsequently to polytetramethylene ether glycol (PTMEG), provides an intermediate used to manufacture spandex products such as LYCRA® fibers. PTMEG is also combined with 1,4-BDO in the production of specialty polyester ethers (COPE). COPEs are high modulus elastomers with excellent mechanical properties and oil/environmental resistance, allowing them to operate at high and low temperature extremes. PTMEG and 1,4-BDO also make thermoplastic polyurethanes processed on standard thermoplastic extrusion, calendaring, and molding equipment, and are characterized by their outstanding toughness and abrasion resistance. The GBL produced from 1,4-BDO provides the feedstock for making pyrrolidones, as well as serving the agrochemical market. The pyrrolidones are used as high performance solvents for extraction processes of increasing use, including for example, in the electronics industry and in pharmaceutical production.

1,4-BDO is produced by two main petrochemical routes with a few additional routes also in commercial operation. One route involves reacting acetylene with formaldehyde, followed by hydrogenation. More recently 1,4-BDO processes involving butane or butadiene oxidation to maleic anhydride, followed by hydrogenation have been introduced. 1,4-BDO is used almost exclusively as an intermediate to synthesize other chemicals and polymers.

Over 25 billion pounds of butadiene (1,3-butadiene, BD) are produced annually and is applied in the manufacture of polymers such as synthetic rubbers and ABS resins, and chemicals such as hexamethylenediamine and 1,4-butanediol. For example, butadiene can be reacted with numerous other chemicals, such as other alkenes, e.g. styrene, to manufacture numerous copolymers, e.g. acrylonitrile 1,3-butadiene styrene (ABS), styrene-1,3-butadiene (SBR) rubber, styrene-1,3-butadiene latex. These materials are used in rubber, plastic, insulation, fiberglass, pipes, automobile and boat parts, food containers, and carpet backing. Butadiene is typically produced as a by-product of the steam cracking process for conversion of petroleum feedstocks such as naphtha, liquefied petroleum gas, ethane or natural gas to ethylene and other olefins. The ability to manufacture butadiene from alternative and/or renewable feedstocks would represent a major advance in the quest for more sustainable chemical production processes.

Crotyl alcohol, also referred to as 2-buten-1-ol, is a valuable chemical intermediate. It serves as a precursor to crotyl halides, esters, and ethers, which in turn are chemical intermediates in the production of monomers, fine chemicals, agricultural chemicals, and pharmaceuticals. Exemplary fine chemical products include sorbic acid, trimethylhydroquinone, crotonic acid and 3-methoxybutanol. Crotyl alcohol is also a precursor to 1,3-butadiene. Crotyl alcohol is currently produced exclusively from petroleum feedstocks. For example Japanese Patent 47-013009 and U.S. Pat. Nos. 3,090,815, 3,090,816, and 3,542,883 describe a method of producing crotyl alcohol by isomerization of 1,2-epoxybutane. The ability to manufacture crotyl alcohol from alternative and/or renewable feedstocks would represent a major advance in the quest for more sustainable chemical production processes.

3-Buten-2-ol (also referenced to as methyl vinyl carbinol (MVC)) is an intermediate that can be used to produce butadiene. There are significant advantages to use of 3-buten-2-ol over 1,3-BDO because there are fewer separation steps and only one dehydration step. 3-Buten-2-ol can also be used as a solvent, a monomer for polymer production, or a precursor to fine chemicals. Accordingly, the ability to manufacture 3-buten-2-ol from alternative and/or renewable feedstock would again present a significant advantage for sustainable chemical production processes.

Adipic acid, a dicarboxylic acid, has a molecular weight of 146.14. It can be used is to produce nylon 6,6, a linear polyamide made by condensing adipic acid with hexamethylenediamine. This is employed for manufacturing different kinds of fibers. Other uses of adipic acid include its use in plasticizers, unsaturated polyesters, and polyester polyols. Additional uses include for production of polyurethane, lubricant components, and as a food ingredient as a flavorant and gelling aid.

Historically, adipic acid was prepared from various fats using oxidation. Some current processes for adipic acid synthesis rely on the oxidation of KA oil, a mixture of cyclohexanone, the ketone or K component, and cyclohexanol, the alcohol or A component, or of pure cyclohexanol using an excess of strong nitric acid. There are several variations of this theme which differ in the routes for production of KA or cyclohexanol. For example, phenol is an alternative raw material in KA oil production, and the process for the synthesis of adipic acid from phenol has been described. The other versions of this process tend to use oxidizing agents other than nitric acid, such as hydrogen peroxide, air or oxygen.

In addition to hexamethylenediamine (HMDA) being used in the production of nylon-6,6 as described above, it is also utilized to make hexamethylene diisocyanate, a monomer feedstock used in the production of polyurethane. The diamine also serves as a cross-linking agent in epoxy resins. HMDA is presently produced by the hydrogenation of adiponitrile.

Caprolactam is an organic compound which is a lactam of 6-aminohexanoic acid (ε-aminohexanoic acid, 6-aminocaproic acid). It can alternatively be considered cyclic amide of caproic acid. One use of caprolactam is as a monomer in the production of nylon-6. Caprolactam can be synthesized from cyclohexanone via an oximation process using hydroxylammonium sulfate followed by catalytic rearrangement using the Beckmann rearrangement process step.

Methylacrylic acid (MAA) is a key precursor of methyl methacrylate (MMA), a chemical intermediate with a global demand in excess of 4.5 billion pounds per year, much of which is converted to polyacrylates. The conventional process for synthesizing methyl methacrylate (i.e., the acetone cyanohydrin route) involves the conversion of hydrogen cyanide (HCN) and acetone to acetone cyanohydrin which then undergoes acid assisted hydrolysis and esterification with methanol to give MAA. Difficulties in handling potentially deadly HCN along with the high costs of byproduct disposal (1.2 tons of ammonium bisulfate are formed per ton of MAA) have sparked a great deal of research aimed at cleaner and more economical processes. As a starting material, MAA can easily be converted into MAA via esterification with methanol.

Thus, there exists a need for the development of methods for effectively producing commercial quantities of bioderived compounds, such as 1,3-BDO, 1,4-BDO, butadiene, crotyl alcohol, MVC, adipate, HMDA, caprolactam, and MAA. The present disclosure satisfies this need and provides related advantages as well.

### SUMMARY OF INVENTION

In some embodiments, provided herein is an engineered phosphoketolase that is a variant of amino acid sequence SEQ ID NO: 1 or 2 or a functional fragment thereof. Such an engineered phosphoketolase includes one or more alterations at a position described in Tables 1, 2, 3, and/or 4. An engineered phosphoketolase described herein is capable of: (a) catalyzing the conversion of fructose-6-phosphate to acetyl-phosphate; (b) catalyzing the conversion of xylulose-5-phosphate to acetyl-phosphate; or (c) catalyzing the conversion of fructose-6-phosphate to acetyl-phosphate and xylulose-5-phosphate to acetyl-phosphate. In some embodiments, an engineered phosphoketolase described herein is capable of forming erythrose-4-phosphate and/or glyceraldehyde-3-phosphate.

In some embodiments, an engineered phosphoketolase described herein has an activity that is at least 0.5, at least 1.0, at least 1.5, or at least 2.0 fold higher than the activity of a wild-type phosphoketolase, such as a phosphoketolase having the amino acid sequence of SEQ ID NO: 1 or 2.

In some embodiments, an engineered phosphoketolase described herein has one or more amino acid alterations that include one or more conservative amino acid substitutions. In some embodiments, an engineered phosphoketolase provided herein has one or more amino acid alterations that include one or more non-conservative amino acid substitutions.

In some embodiments, an engineered phosphoketolase described herein has one or more amino acid alteration described in Table 1.

In some embodiments, an engineered phosphoketolase described herein has one or more amino acid alteration described in Table 2.

In some embodiments, an engineered phosphoketolase provided herein has at least two, three, four, five, six, seven, eight, nine or ten amino acid alterations described herein (e.g., Tables 1, 2, 3, and/or 4).

In some embodiments, provided herein is a recombinant nucleic acid encoding an engineered phosphoketolase described herein. In some embodiments, such a recombinant nucleic acid has a nucleotide sequence encoding an engineered phosphoketolase described herein operatively linked to a promoter. In some embodiments, also provided herein is a vector having such recombinant nucleic acid.

In some embodiments, provided herein is a non-naturally occurring microbial organism having a recombinant nucleic acid encoding an engineered phosphoketolase described herein. Such a microbial organism, in some embodiments, further includes an exogenous nucleic acid encoding: (a) an acetate kinase and an acetyl-CoA transferase, synthetase, or ligase, wherein the acetate kinase catalyzes the conversion of acetyl-phosphate to acetate and the acetyl-CoA transferase, synthetase, or ligase catalyzes the conversion of acetate to acetyl-CoA; (b) a phosphotransacetylase, wherein the phosphotransacetylase catalyzes the conversion of acetyl-phosphate to acetyl-CoA; (c) a pyruvate dehydrogenase, a pyruvate ferredoxin oxidoreductase, or a pyruvate:NADP+ oxidoreductase, wherein the pyruvate dehydrogenase, the pyruvate ferredoxin oxidoreductase, or the pyruvate:NADP+ oxidoreductase catalyzes the conversion of pyruvate to acetyl-CoA and carbon dioxide; or (d) a pyruvate formate lyase, wherein the pyruvate formate lyase catalyzes the conversion of pyruvate to acetyl-CoA and formate. Alternatively, or in addition, such a microbial organism, in some embodiments, further includes exogenous nucleic acids encoding a combination of enzymes that catalyze the conversion of glyceraldehyde-3-phosphate to pyruvate, wherein the combination of enzymes include a glyceraldehyde-3-phosphate dehydrogenase, a phosphoglycerate kinase, a phosphoglyceromutase, an enolase, a pyruvate kinase and a PTS-dependent substrate import.

In some embodiments, a microbial organism described herein includes an exogenous nucleic acid that is heterologous to the microbial organism. In some embodiments, a microbial organism described herein includes an exogenous nucleic acid that is homologous to the microbial organism.

In some embodiments, a microbial organism described herein includes a pathway capable of producing a bioderived compound from acetyl-CoA. Such a bioderived compound, in some embodiments, is an alcohol, a glycol, an organic acid, an alkene, a diene, an organic amine, an organic aldehyde, a vitamin, a nutraceutical or a pharmaceutical. Examples of alcohols include: (a) a biofuel alcohol, wherein said biofuel is a primary alcohol, a secondary alcohol, a diol or triol comprising C3 to C10 carbon atoms; (b) n-propanol or isopropanol; and (c) a fatty alcohol, wherein said fatty alcohol comprises C4 to C27 carbon atoms, C8 to C18 carbon atoms, C12 to C18 carbon atoms, or C12 to C14 carbon atoms. Examples of biofuel alcohols include: 1-propanol, isopropanol, 1-butanol, isobutanol, 1-pentanol, isopentenol, 2-methyl-1-butanol, 3-methyl-1-butanol, 1-hexanol, 3-methyl-1-pentanol, 1-heptanol, 4-methyl-1-hexanol, and 5-methyl-1-hexanol. In some embodiments, the diol is a propanediol or a butanediol, such as 1,4 butanediol, 1,3-butanediol or 2,3-butanediol. In some embodiments, the bioderived compound is selected from the group consisting of: (a) 1,4-butanediol or an intermediate thereto, wherein said intermediate is optionally 4-hydroxybutanoic acid (4-HB); (b) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol; (c) 1,3-butanediol or an intermediate thereto, wherein said intermediate is optionally 3-hydroxybutyrate (3-HB), 2,4-pentadienoate, crotyl alcohol or 3-buten-1-ol; (d) adipate, 6-aminocaproic acid, caprolactam, hexamethylenediamine, levulinic acid or an intermediate thereto, wherein said intermediate is optionally adipyl-CoA or 4-aminobutyryl-CoA; (e) methacrylic acid or an ester thereof, 3-hydroxyisobutyrate, 2-hydroxyisobutyrate, or an intermediate thereto, wherein said ester is optionally methyl methacrylate or poly(methyl methacrylate); (f) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto; (g) succinic acid or an intermediate thereto; and (h) a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

In some embodiments, a microbial organism described herein is in a substantially anaerobic culture medium.

In some embodiments, a microbial organism described herein is a species of bacteria, yeast, or fungus.

In some embodiments, a microbial organism described herein is capable of producing at least 10% more acetyl-phosphate, acetyl-CoA or a bioderived compound compared to a control microbial organism that does not include a recombinant nucleic acid encoding an engineered phosphoketolase described herein.

In some embodiments, provided herein is a method for producing a bioderived compound described herein that includes culturing a non-naturally occurring microbial organism described herein under conditions and for a sufficient period of time to produce the bioderived compound. Such a method, in some embodiments, also includes separating the bioderived from other components in the culture. Methods for performing such separating includes extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, provided herein is culture medium having the bioderived compound produced by a method provided herein, wherein the bioderived compound has a carbon-12, carbon-13 and carbon-14 isotope ratio that reflects an atmospheric carbon dioxide uptake source.

In some embodiments, provided herein is a bioderived compound produced according to a method described herein. Such a bioderived compound, in some embodiments, has an Fm value of at least 80%, at least 85%, at least 90%, at least 95% or at least 98%.

In some embodiments, provided herein is composition having a bioderived compound described herein and a compound other than the bioderived compound. Such a compound other than said bioderived compound, in some embodiments, is a trace amount of a cellular portion of a non-naturally occurring microbial organism having a bioderived compound pathway.

In some embodiments, provided herein is composition having a bioderived compound described herein of, or a cell lysate or culture supernatant thereof.

### BRIEF DESCRIPTION OF THE DRAWINGS

FIG. 1 shows an alignment of the fructose-6-phosphate phosphoketolase from Bifidobacterium adolescentis (SEQ ID NO: 1; also referred to as H2) and the xylulose-5-phosphate/fructose-6-phosphate phosphoketolase from Collinsella aerofaciens (SEQ ID NO: 2; also referred to as D3). A consensus sequence (SEQ ID NO: 5) is also depicted.

FIG. 2 provides a graph showing correlation between D-xylulose-5-phosphate phosphoketolase (XPK) activity and D-fructose-6-phosphate phosphoketolase (FPK) activity for each single variant hit from primary screening. The two sets refer to the two screening rounds.

FIG. 3 shows a box plot comparison for FPK activity from a variant screening library. Results for one negative control, two positive controls, and library variants are shown.

FIG. 4 shows a box plot comparison for XPK activity from a variant screening library. Results for one negative control, two positive controls, and library variants are shown.

### DETAILED DESCRIPTION DESCRIBED HEREIN

The subject matter described herein relates to enzyme variants that have desirable properties and are useful for producing desired products (e.g., acetyl-phosphate, acetyl-CoA or a bioderived compound). In some embodiments, the subject matter described herein relates to engineered phosphoketolases, which are enzyme variants that have markedly different structural and/or functional characteristics compared to a wild-type phosphoketolase that occurs in nature. Thus, the engineered phosphoketolases provided herein are not naturally occurring enzymes. Such engineered phosphoketolases provided are useful in an engineered cell, such as a microbial organism, that has been engineered to produce a desired product (e.g., acetyl-phosphate, acetyl-CoA or a bioderived compound). For example, as disclosed herein, a cell, such as a microbial organism, having a metabolic pathway can produce a desired product (e.g., acetyl-phosphate, acetyl-CoA or a bioderived compound). Engineered phosphoketolases having desirable characteristics as described herein can be introduced into a cell, such as microbial organism, that has a metabolic pathway that uses phosphoketolase activity to produce a desired product (e.g., acetyl-phosphate, acetyl-CoA or a bioderived compound). Thus, the engineered phosphoketolases provided herein can be utilized in engineered cells, such as microbial organisms, to produce a desired product.

### Conventions and Abbreviations

| Abbreviation | Convention |
|---|---|
| Ala; A | Alanine |
| Arg; R | Arginine |
| Asn; N | Asparagine |
| Asp; D | Aspartic acid |
| Cys; C | Cysteine |
| Glu; E | Glutamic acid |
| Gln; Q | Glutamine |
| Gly; G | Glycine |
| His; H | Histidine |
| Ile; I | Isoleucine |
| Leu; L | Leucine |
| Lys; K | Lysine |
| Met; M | Methionine |
| Phe; F | Phenylalanine |
| Pro; P | Proline |
| Ser; S | Serine |
| Thr; T | Threonine |
| Trp; W | Tryptophan |
| Tyr; Y | Tyrosine |
| Val; V | Valine |
| Xu5P | Xylulose-5-phosphate |
| α-GDH | α-glycerophosphate-3-phosphate dehydrogenase |
| E4P | D-erythrose-4-phosphate |
| E4PDH | D-erythrose-4-phosphate dehydrogenase |
| F6P | D-fructose-6-phosphate |
| FPK | D-fructose-6-phosphate phosphoketolase |
| GAP | D-glyceraldehyde-3-phosphate |
| NAD+ | Nicotinamide adenine dinucleotide |
| NADH | Nicotinamide adenine dinucleotide hydride |
| Pi | Phosphate |
| PK | Phosphoketolase |
| TPI | Triosephosphate isomerase |
| Xu5P | D-xylulose-5-phosphate |
| XPK | D-xylulose-5-phosphate phosphoketolase |

As used herein the term “about” means±10% of the stated value. The term “about” can mean rounded to the nearest significant digit. Thus, about 5% means 4.5% to 5.5%. Additionally, about in reference to a specific number also includes that exact number. For example, about 5% also includes exact 5%.

As used herein, the term “alteration” or grammatical equivalents thereof when used in reference to any peptide, polypeptide, protein, nucleic acid or polynucleotide described herein refers to a change in structure of an amino acid residue or nucleic acid base relative to the starting or reference residue or base. An alteration of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. An alteration of a nucleic acid base includes, for example, changing one naturally occurring base for a different naturally occurring base, such as changing an adenine to a thymine or a guanine to a cytosine or an adenine to a cytosine or a guanine to a thymine. An alteration of a nucleic acid base may result in an alteration of the encoding peptide, polypeptide or protein by changing the encoded amino acid residue or function of the peptide, polypeptide or protein. An alteration of a nucleic acid base may not result in an alteration of the amino acid sequence or function of encoded peptide, polypeptide or protein, also known as a silent mutation.

As used herein, the term “bioderived” means derived from or synthesized by a biological organism and can be considered a renewable resource since it can be generated by a biological organism. Such a biological organism, in particular the non-naturally occurring microbial organism disclosed herein, can utilize feedstock or biomass, such as, sugars (e.g., cellobiose, glucose, fructose, xylose, galactose (e.g., galactose from marine plant biomass), and sucrose), carbohydrates obtained from an agricultural, plant, bacterial, or animal source, and glycerol (e.g., crude glycerol byproduct from biodiesel manufacturing) for synthesis of a desired bioderived compound.

As used herein, the term “CoA” or “coenzyme A” is intended to mean an organic cofactor or prosthetic group (nonprotein portion of an enzyme) whose presence is required for the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “conservative substitution” refers to the replacement of one amino acid for another such that the replacement takes place within a family of amino acids that are related in their side chains. Alternatively, the term “non-conservative substitution” refers to the replacement of one amino acid residue for another such that the replaced residue is going from one family of amino acids to a different family of residues. Genetically encoded amino acids can be divided into four families: (1) acidic (negatively charged)=Asp (D), Glu (G); (2) basic (positively charged)=Lys (K), Arg (R), His (H); (3) non-polar (hydrophobic)=Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic=Ala (A), Val (V), Leu (L), Ile (I), Met (M), Phe (F); and (ii) moderately hydrophobic=Gly (G), Pro (P), Cys (C), Tyr (Y), Trp (W); and (4) uncharged polar=Asn (N), Gln (Q), Ser (S), Thr (T). In alternative fashion, the amino acid repertoire can be grouped as (1) acidic (negatively charged)=Asp (D), Glu (G); (2) basic (positively charged)=Lys (K), Arg (R), His (H), and (3) aliphatic=Gly (G), Ala (A), Val (V), Leu (L), Ile (I), Ser (S), Thr (T), with Ser (S) and Thr (T) optionally being grouped separately as aliphatic-hydroxyl; (4) aromatic=Phe (F), Tyr (Y), Trp (W); (5) amide=Asn (N), Glu (Q); and (6) sulfur-containing=Cys (C) and Met (M) (see, for example, Biochemistry, 4th ed., Ed. by L. Stryer, WH Freeman and Co., 1995, which is incorporated by reference herein in its entirety).

As used herein, the term “culture medium,” “medium,” “growth medium” or grammatical equivalents thereof refers to a liquid or solid (e.g., gelatinous) substance containing nutrients that supports the growth of a cell, including a microbial organism, such as the microbial organism described herein. Nutrients that support growth include, but are not limited to, the following: a substrate that supplies carbon, such as, but are not limited to, cellobiose, galactose, glucose, xylose, ethanol, acetate, arabinose, arabitol, sorbitol and glycerol; salts that provide essential elements including magnesium, nitrogen, phosphorus, and sulfur; a source for amino acids, such as peptone or tryptone; and a source for vitamin content, such as yeast extract. Culture medium can be a defined medium, in which quantities of all ingredients are known, or an undefined medium, in which the quantities of all ingredients are not known. Culture medium can also include substances other than nutrients needed for growth, such as a substance that only allows select cells to grow (e.g., antibiotic or antifungal), which are generally found in selective medium, or a substance that allows for differentiation of one microbial organism over another when grown on the same medium, which are generally found in differential or indicator medium. Such substances are well known to a person skilled in the art.

As used herein, the term “engineered” or “variant” when used in reference to any peptide, polypeptide, protein, nucleic acid or polynucleotide described herein refers to a sequence of amino acids or nucleic acids having at least one alteration at an amino acid residue or nucleic acid base as compared to a parent sequence. The parent sequence of amino acids or nucleic acids can be, for example, a wild-type sequence or a homolog thereof, or a modified variant of a wild-type sequence or homolog thereof.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid described herein can utilize either or both a heterologous or homologous encoding nucleic acid.

It is understood that, when more than one recombinant nucleic acid and/or exogenous nucleic acid is included into a microbial organism, the more than one recombinant nucleic acid and/or exogenous nucleic acid refers to the referenced encoding nucleic acid or biosynthetic activity, as discussed herein. It is further understood, as disclosed herein, that such more than one recombinant nucleic acids or exogenous nucleic acids can be introduced into the host microbial organism on separate nucleic acid molecules, on polycistronic nucleic acid molecules, or a combination thereof, and still be considered as more than one recombinant nucleic acid and/or exogenous nucleic acid. For example, as disclosed herein a microbial organism can be engineered to express two or more recombinant and/or exogenous nucleic acids encoding a desired pathway enzyme or protein. In the case where two recombinant and/or exogenous nucleic acids encoding an enzyme or protein having a desired activity are introduced into a host microbial organism, it is understood that the two recombinant and/or exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two recombinant and/or exogenous nucleic acids can be introduced into a host organism in any desired combination, for example, on a single plasmid, on separate plasmids, can be integrated into the host chromosome at a single site or multiple sites, and still be considered as two or more recombinant or exogenous nucleic acids, for example three exogenous nucleic acids. Thus, the number of referenced recombinant or exogenous nucleic acids or biosynthetic activities refers to the number of encoding nucleic acids or the number of biosynthetic activities, not the number of separate nucleic acids introduced into the host organism.

The term “Fm value” or “Fraction Modem value” when used in reference to a compound is a ratio of carbon-14 (¹⁴C) to carbon-12 (¹²C). Specifically, Fm value is computed from the expression: Fm=(S−B)/(M−B), where B, S and M represent the ¹⁴C/¹²C ratios of the blank, the sample and the modern reference, respectively. Fm value is a measurement of the deviation of the ¹⁴C/¹²C ratio of a sample from “Modem.” Modem is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for 613. An Fm=0% represents the entire lack of carbon-14 atoms in a material, thus indicating a fossil (for example, petroleum based) carbon source, whereas a Fm=100%, after correction for the post-1950 injection of carbon-14 into the atmosphere from nuclear bomb testing, indicates an entirely modern carbon source. The percent modern carbon (pMC) can be greater than 100% because of the continuing but diminishing effects of the 1950s nuclear testing programs, which resulted in a considerable enrichment of carbon-14 in the atmosphere. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old. Applications of carbon-14 dating techniques to quantify bio-based content of materials are well known in the art (see, e.g., Currie et al., Nuclear Instruments and Methods in Physics Research B, 172:281-287 (2000), and Colonna et al., Green Chemistry, 13:2543-2548 (2011)).

As used herein, the term “functional fragment” when used in reference to a peptide, polypeptide or protein is intended to refer to a portion of the peptide, polypeptide or protein that retains some or all of the activity (e.g., catalyzing the conversion of fructose-6-phosphate to acetyl-phosphate and/or xylulose-5-phosphate to acetyl-phosphate) of the original peptide, polypeptide or protein from which the fragment was derived. Such functional fragments include amino acid sequences that are about 500 to about 800, about 500 to about 775, about 500 to about 750, about 500 to about 725, about 500 to about 700, about 600 to about 800, about 600 to about 775, about 600 to about 750, about 600 to about 725, about 600 to about 700, about 700 to about 800, about 700 to about 775, about 700 to about 750, about 700 to about 725, about 750 to about 800, about 750 to about 775, about 725 to about 800, about 725 to about 775, about 725 to about 750 amino acids in length. These functional fragments can, for example, be truncations (e.g., C-terminal or N-terminal truncations) of a peptide, polypeptide, or protein. Functional fragments can also include one or more amino acid alteration described herein, such as an amino acid alteration of an engineered peptide described herein.

As used herein, the term “isolated” when used in reference to a molecule (e.g., peptide, polypeptide, protein, nucleic acid, polynucleotide, vector) or a cell (e.g., a yeast cell) refers to a molecule or cell that is substantially free of at least one component as the referenced molecule or cell is found in nature. The term includes a molecule or cell that is removed from some or all components as it is found in its natural environment. Therefore, an isolated molecule or cell can be partly or completely separated from other substances as it is found in nature or as it is grown, stored or subsisted in non-naturally occurring environments.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “non-naturally occurring” when used in reference to a microbial organism described herein is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, genetic alterations within coding regions and functional fragments thereof. Additional modifications include, for example, non-coding regulatory regions in which the modifications alter expression of a gene or operon. Exemplary metabolic polypeptides include enzymes or proteins within an acetyl-CoA or bioderived compound pathway described herein.

As use herein, the term “operatively linked” when used in reference to a nucleic acid encoding an engineered phosphoketolase refers to connection of a nucleotide sequence encoding an engineered phosphoketolase described herein to another nucleotide sequence (e.g., a promoter) is such a way as to allow for the connected nucleotide sequences to function (e.g., express the engineered phosphoketolase in the microbial organism).

As used herein, the term “pathway” when used in reference to production of a desired product (e.g., acetyl-phosphate, acetyl-CoA or a bioderived compound) refers to one or more polypeptides (e.g., proteins or enzymes) that catalyze the conversion of a substrate compound to a product compound and/or produce a co-substrate for the conversion of a substrate compound to a product compound. Such a product compound can be one of the bioderived compounds described herein, or an intermediate compound that can lead to the bioderived compound upon further conversion by other proteins or enzymes of the metabolic pathway. Accordingly, a metabolic pathway can be comprised of a series of metabolic polypeptides (e.g., two, three, four, five, six, seven, eight, nine, ten or more) that act upon a substrate compound to convert it to a given product compound through a series of intermediate compounds. The metabolic polypeptides of a metabolic pathway can be encoded by an exogenous nucleic acid as described herein or produced naturally by the host microbial organism.

As used herein, the term “recombinant” with respect to a nucleic acid, such as a nucleic acid comprising a gene that encodes a protein or polypeptide (e.g., an engineered phosphoketolase described herein), refers to: a nucleic acid that has been artificially supplied to a biological system; a nucleic acid that has been modified within a biological system, or a nucleic acid whose expression or regulation has been manipulated within a biological system. The recombinant nucleic acid can be supplied to the biological system, for example, by introduction of the nucleic acid into genetic material of a microbial organism, such as by integration into a microbial organism chromosome, or as non-chromosomal genetic material such as a plasmid. A recombinant nucleic acid that is introduced into or expressed in a microbial organism may be a nucleic acid that comes from a different organism or species from the microbial organism, or may be a synthetic nucleic acid, or may be a nucleic acid that is also endogenously expressed in the same organism or species as the microbial organism. A recombinant nucleic acid that is also endogenously expressed in the same organism or species as the microbial organism can be considered heterologous if: the sequence of the recombinant nucleic acid is modified relative to the endogenously expressed sequence, the sequence of a regulatory region such as a promoter that controls expression of the nucleic acid is modified relative to the regulatory region of the endogenously expressed sequence, the nucleic acid is expressed in an alternate location in the genome of the microbial organism relative to the endogenously expressed sequence, the nucleic acid is expressed in a different copy number in the microbial organism relative to the endogenously expressed sequence, and/or the nucleic acid is expressed as non-chromosomal genetic material such as a plasmid in the microbial organism.

As used herein, the term “promoter” when used in reference to a nucleic acid encoding an engineered phosphoketolase refers to a nucleotide sequence where transcription of a linked open reading frame (e.g., a nucleotide sequence encoding an engineered phosphoketolase) by an RNA polymerase begins. A promoter sequence can be located directly upstream or at the 5′ end of the transcription initiation site. RNA polymerase and the necessary transcription factors bind to a promoter sequence and initiate transcription. Promoter sequences define the direction of transcription and indicate which DNA strand will be transcribed, i.e. the sense strand.

As used herein, the term “substantially anaerobic” when used in reference to a culture or growth condition is intended to mean that the amount of dissolved oxygen in a liquid medium is less than about 10% of saturation. The term also is intended to include sealed chambers maintained with an atmosphere of less than about 1% oxygen that include liquid or solid medium.

As used herein, the term “vector” refer to a compound and/or composition that transduces, transforms, or infects a microbial organism, thereby causing the microbial organism to express nucleic acids and/or proteins other than those native to the microbial organism, or in a manner not native to the cell. Vectors can be constructed to include one or more biosynthetic pathway enzyme or protein, such as an engineered phosphoketolase described herein, encoded by a nucleotide sequence operably linked to expression control sequences (e.g., promoter) that are functional in the microbial organism (“expression vector”). Expression vectors applicable for use in the microbial organisms described herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more recombinant or exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of a recombinant or exogenous nucleic acid encoding an enzyme or protein involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid or its corresponding gene product (e.g., enzyme or protein). It is understood by those skilled in the art that the recombinant or exogenous nucleic acid is expressed in a sufficient amount to produce the desired product, and it is further understood that expression levels can be optimized to obtain sufficient expression using methods well known in the art and as disclosed herein.

Those skilled in the art will understand that the genetic alterations, including metabolic modifications exemplified herein, are described with reference to a suitable microbial organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microbial organism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms described herein having biosynthetic capability for a desired product, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microbial organism that encode an enzyme catalyzing a similar or substantially similar metabolic reaction, those skilled in the art also can utilize these evolutionally related genes. Similarly for a gene disruption, evolutionally related genes can also be disrupted or deleted in a microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5 1999) and the following parameters: Matrix: 0 BLOSUM62; gap open: 11; gap extension: 1; x_dropoff: 50; expect: 10.0; wordsize: 3; filter: on. Nucleotide sequence alignments can be performed using BLASTN version 2.0.6 (Sep. 16 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In some embodiments, provided herein is an engineered phosphoketolase that is a variant of a wild-type or parent phosphoketolase. Such an engineered phosphoketolase includes one or more alterations described herein and higher catalytic activity relative to the wild-type or parent phosphoketolase as described herein. The engineered phosphoketolase provided herein is capable of catalyzing the conversion of fructose-6-phosphate to acetyl-phosphate and/or xylulose-5-phosphate to acetyl-phosphate. Accordingly, in some embodiments, the engineered phosphoketolase provided herein is capable of catalyzing the conversion of fructose-6-phosphate to acetyl-phosphate. In some embodiments, an engineered phosphoketolase is a D-fructose-6-phosphate phosphoketolase (FPK). In some embodiments, an engineered phosphoketolase is a D-xylulose-5-phosphate phosphoketolase (XPK) In some embodiments, the engineered phosphoketolase provided herein is capable of catalyzing the conversion of xylulose-5-phosphate to acetyl-phosphate. In some embodiments, the engineered phosphoketolase provided herein is capable of catalyzing the conversion of fructose-6-phosphate to acetyl-phosphate and xylulose-5-phosphate to acetyl-phosphate. Such an engineered phosphoketolase provided herein can be classified as an enzyme that catalyzes the reaction EC 4.1.2.22 (fructose-6-phosphate phosphoketolase) and/or EC 4.1.2.9 (phosphoketolase). Accordingly, in some embodiments, an engineered phosphoketolase provided herein is capable of forming erythrose-4-phosphate and/or glyceraldehyde-3-phosphate. Exemplary enzymatic conversions of an engineered phosphoketolase provided herein include, but are not limited to, the conversion of fructose-6-phosphate to acetyl-phosphate and erythrose-4-phosphate, and/or the conversion of xylulose-5-phosphate to acetyl-phosphate and glyceraldehyde-3-phosphate.

Exemplary enzymatic reactions catalyzed by an engineered phosphoketolase described herein is represented by:

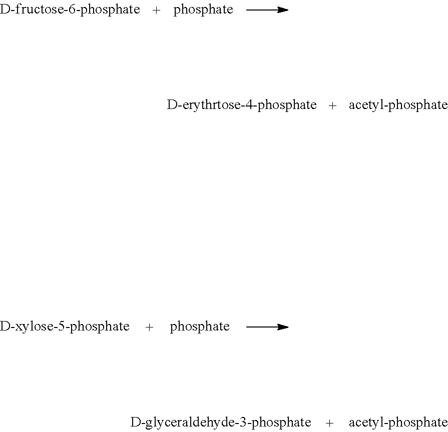

The active site of a phosphoketolase may be defined by generating the three-dimensional structure of the phosphoketolase and identifying the residues within a particular distance of a docked substrate. As used in this disclosure, a residue is within the active site of a phosphoketolase if it is within about 12 angstroms (Å) of a substrate docking site within the phosphoketolase.

As a non-limiting example, the residues at the following positions in SEQ ID NO: 1 are located within 12 angstroms (Å) from a docked fructose-6-phosphate: 63, 64, 65, 68, 98, 142, 143, 144, 156, 158, 219, 220, 221, 301, 322, 323, 436, 437, 438, 440, 441, 442, 443, 478, 499, 502, 504, 505, 540, 542, 546, 547, 548, 549, 550, 551, 553, 606, and 607 and may be considered part of the active site. Residues at positions that are not within 12 A from a docked fructose-6-phosphate (e.g., positions 38, 45, 49, 53, 60, 76, 77, 79, 87, 92, 103, 104, 117, 119, 122, 136, 171, 174, 176, 178, 194, 200, 201, 208, 235, 239, 248, 253, 254, 255, 257, 267, 276, 283, 287, 291, 292, 305, 308, 335, 339, 364, 365, 367, 372, 374, 377, 383, 391, 400, 403, 405, 407, 420, 447, 449, 462, 463, 468, 475, 483, 524, 526, 536, 559, 560, 567, 571, 572, 580, 582, 584, 593, 601, 611, 621, 627, 628, 641, 642, 648, 657, 662, 664, 665, 666, 670, 671, 679, 687, 688, 689, 712, 716, 731, 745, 757, 759, 765, 767, 771, 774, 776, 781, 786, 788, 794, 799, 804, and 815 in SEQ ID NO: 1) may not be considered part of the active site.

As a non-limiting example, the residues at the following positions in SEQ ID NO: 2 are located within 12 Å from a docked fructose-6-phosphate: 72, 73, 74, 107, 151, 152, 153, 165, 166, 167, 170, 228, 229, 230, 315, 336, 426, 449, 450, 451,453, 454, 455, 456, 492, 513, 516, 518, 519, 556, 560, 561, 562, 563, 564, 565, 567, 617, 619, and 620 and may be considered part of the active site. Residues at positions that are not within 12 Å from a docked fructose-6-phosphate (e.g., positions 4, 10, 14, 15, 16, 19, 26, 31, 43, 47, 49, 54, 59, 63, 65, 69, 85, 86, 88, 96, 112, 113, 115, 117, 123, 126, 131, 145, 183, 195,209,210,217,238,258,261,271,276,285,290,294,300,302, 306, 319, 322, 330, 346, 349, 354, 378, 379, 386, 391, 407, 411, 420, 431, 433, 443, 460, 462, 474, 481, 484, 487, 503, 510, 531, 549, 581, 582, 585, 586, 588, 591, 597, 604, 606, 611, 614, 616, 624, 626, 634, 640, 641, 648, 651, 652, 654, 658, 660, 675, 681, 684, 694, 696, 697, 698, 700, 701, 702, 704, 708, 709, 710, 714, 715, 716, 717, 726, 728, 736, 743, 770, 771, 779, 781, 783, 785, 788, 792, 795, 800, 802, 804, 808, 810, 812, 813, and 836 in SEQ ID NO: 2) may not be considered part of the active site.

An engineered phosphoketolase provided herein can be used to produce a desired bioderived compound, such as an alcohol, a glycol, an organic acid, an alkene, a diene, an organic amine, an organic aldehyde, a vitamin, a nutraceutical or a pharmaceutical, or other desired products, from acetyl-CoA in a cell, such as a microbial organism, containing a suitable metabolic pathway. For example, an engineered phosphoketolase provided herein can be used to produce acetyl-phosphate, which is then converted to acetyl-CoA using: (a) an acetate kinase and an acetyl-CoA transferase, synthetase, or ligase, wherein the acetate kinase catalyzes the conversion of acetyl-phosphate to acetate and the acetyl-CoA transferase, synthetase, or ligase catalyzes the conversion of acetate to acetyl-CoA; or (b) a phosphotransacetylase, wherein the phosphotransacetylase catalyzes the conversion of acetyl-phosphate to acetyl-CoA.

Exemplary reactions that an acetate kinase and an acetyl-CoA transferase, synthetase, or ligase catalyze to form acetyl-CoA using acetyl-phosphate, a product of an engineered phosphoketolase described herein, is represented by:
acetate kinase:

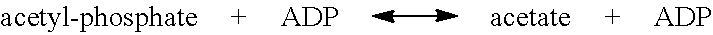

acetyl-CoA transferase, synthetase, or ligase:

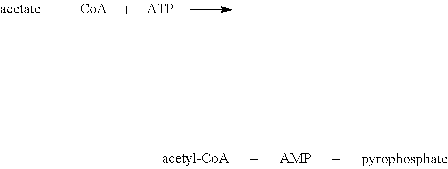

An exemplary reaction that a phosphotransacetylase catalyzes is the acetylation of a CoA to form acetyl-CoA using the substrate acetyl-phosphate, a product of an engineered phosphoketolase described herein, which represented by:

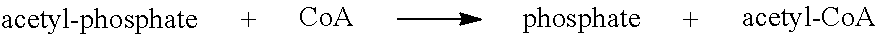

Alternatively or in addition, an engineered phosphoketolase provided herein can be used to produce erythrose-4-phosphate and/or glyceraldehyde-3-phosphate. The glyceraldehyde-3-phosphate produced by an engineered phosphoketolase provided herein can then be converted to acetyl-CoA via pyruvate using: a combination of enzymes that comprises a glyceraldehyde-3-phosphate dehydrogenase, a phosphoglycerate kinase, a phosphoglyceromutase, an enolase, a pyruvate kinase and a PTS-dependent substrate import, which produce pyruvate. The pyruvate produced by such a combination of enzymes can be converted to acetyl-CoA using: (a) a pyruvate dehydrogenase, a pyruvate ferredoxin oxidoreductase, or a pyruvate:NADP+ oxidoreductase, wherein the pyruvate dehydrogenase, the pyruvate ferredoxin oxidoreductase, or the pyruvate:NADP+ oxidoreductase catalyzes the conversion of pyruvate to acetyl-CoA and carbon dioxide; or (b) a pyruvate formate lyase, wherein the pyruvate formate lyase catalyzes the conversion of pyruvate to acetyl-CoA and formate. The acetyl-CoA produced by these combinations of enzymes can be used as the starting material for production of a bioderived compound, wherein the microbial organism further includes a pathway capable of producing a bioderived compound from acetyl-CoA. Thus, the engineered phosphoketolases provided herein are particularly useful to provide an improved enzymatic route and microbial organism to product a desired product (e.g., acetyl-phosphate, acetyl-CoA or a bioderived compound).

In some embodiments, provided herein is an engineered phosphoketolase comprising a variant of amino acid sequence SEQ ID NO:1 or 2, wherein the engineered phosphoketolase comprises one or more alterations at a position described in Tables 1, 2, 3, and/or 4. In some embodiments, the engineered phosphoketolase comprises one or more alterations at a position described in Table 1. In some embodiments, the engineered phosphoketolase comprises one or more alterations at a position described in Table 2. In some embodiments, the engineered phosphoketolase comprises one or more alterations at a position described in Table 3. In some embodiments, the engineered phosphoketolase comprises one or more alterations at a position described in Table 4.

In some embodiments, an engineered phosphoketolase provided herein is capable of: (a) catalyzing the conversion of fructose-6-phosphate to acetyl-phosphate; (b) catalyzing the conversion of xylulose-5-phosphate to acetyl-phosphate; or (c) catalyzing the conversion of fructose-6-phosphate to acetyl-phosphate and xylulose-5-phosphate to acetyl-phosphate. Accordingly, in some embodiments, an engineered phosphoketolase provided herein catalyzes the conversion of fructose-6-phosphate to acetyl-phosphate. In some embodiments, an engineered phosphoketolase provided herein catalyzes the conversion of xylulose-5-phosphate to acetyl-phosphate. In some embodiments, an engineered phosphoketolase provided herein catalyzes the conversion of fructose-6-phosphate to acetyl-phosphate and xylulose-5-phosphate to acetyl-phosphate.

Depending upon whether an engineered phosphoketolase provided herein catalyzes the conversion of fructose-6-phosphate or xylulose-5-phosphate, an engineered phosphoketolase provided herein is also capable of producing erythrose-4-phosphate and/or glyceraldehyde-3-phosphate. Accordingly, in some embodiments, an engineered phosphoketolase provided herein catalyzes the conversion of fructose-6-phosphate to acetyl-phosphate and erythrose-4-phosphate. In some embodiments, an engineered phosphoketolase that catalyzes conversion of fructose-6-phosphate to acetyl-phosphate and erythrose-4-phosphate is referred to as a D-fructose-6-phosphate phosphoketolase or fructose-6-phosphate phosphoketolase (FPK). In some embodiments, an engineered phosphoketolase provided herein catalyzes the conversion of xylulose-5-phosphate to acetyl-phosphate and glyceraldehyde-3-phosphate. In some embodiments, an engineered phosphoketolase that catalyzes conversion of xylulose-5-phosphate to acetyl-phosphate and glyceraldehyde-3-phosphate is referred to as a D-xylulose-5-phosphate phosphoketolase or xylulose-5-phosphate phosphoketolase (XPK). In some embodiments, an engineered phosphoketolase provided herein catalyzes the conversion of fructose-6-phosphate to acetyl-phosphate and erythrose-4-phosphate as well as xylulose-5-phosphate to acetyl-phosphate and glyceraldehyde-3-phosphate. In some embodiments, an engineered phosphoketolase that catalyzes conversion of fructose-6-phosphate to acetyl-phosphate and erythrose-4-phosphate as well as xylulose-5-phosphate to acetyl-phosphate and glyceraldehyde-3-phosphate is a FPK and a XPK.

It is understood that the engineered phosphoketolases, such as polypeptide variants of phosphoketolases having the amino acid sequence of SEQ ID NO: 1 or SEQ ID NO: 2, as described herein, can carry out a similar enzymatic reaction as the parent phosphoketolase as discussed above. It is further understood that the polypeptide variants of the phosphoketolase enzyme can include variants that provide a beneficial characteristic to the engineered phosphoketolase, including but not limited to, increased activity (see, e.g., Example 1). In some embodiments, the engineered phosphoketolase can exhibit an activity that is at least the same or higher than a wild-type or parent phosphoketolase, that is, it has activity that is higher than a phosphoketolase without the variant at the same amino acid position(s). For example, the engineered phosphoketolases provided here can have at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, at least 10, or even higher fold activity over a wild-type or parent phosphoketolase (see, e.g., Example 1). In some embodiments, an engineered phosphoketolase provided herein has an activity that is at least 0.5, at least 1.0, at least 1.5, or at least 2.0 fold higher than the activity of a phosphoketolase consisting of the amino acid sequence of SEQ ID NO: 1 or 2. In some embodiments, an engineered phosphoketolase provided herein has an activity that is at least 0.5 fold higher. In some embodiments, an engineered phosphoketolase provided herein has an activity that is at least 1.0 fold higher. In some embodiments, an engineered phosphoketolase provided herein has an activity that is at least 1.5 fold higher. In some embodiments, an engineered phosphoketolase provided herein has an activity that is at least 2.0 fold higher. It is understood that activity refers to the ability of an engineered phosphoketolase described herein to convert a substrate to a product relative to a wild-type or parent phosphoketolase under the same assay conditions, such as those described herein (see, e.g., Example 1).

In some embodiments, the activity of a phosphoketolase described herein is measured as the catalytic constant (k_{cat}) value or turnover number. In some embodiments, the k_{cat} is at least 0.1 s⁻¹, at least 0.2 s⁻¹, at least 0.3 s⁻¹, at least 0.4 s⁻¹, at least 0.5 s⁻¹, at least 0.6 s⁻¹, at least 0.7 s⁻¹, at least 0.8 s⁻¹, at least 0.9 s⁻¹, at least 1 s⁻¹, at least 2 s⁻¹, at least 3 s⁻¹, at least 4 s⁻¹, at least 5 s⁻¹, at least 6 s⁻¹, at least 7 s⁻¹, at least 8 s⁻¹, at least 9 s⁻¹, at least 10 s⁻¹, at least 11 s⁻¹, at least 12 s⁻¹, at least 13 s⁻¹, at least 14 s⁻¹, at least 15 s⁻¹, at least 16 s⁻¹, at least 17 s⁻¹, at least 18 s⁻¹, at least 19 s⁻¹, at least 20 s⁻¹, at least 21 s⁻¹, at least 22 s⁻¹, at least 23 s⁻¹, at least 24 s⁻¹, at least 25 s⁻¹, at least 26 s⁻¹, at least 27 s⁻¹, at least 28 s⁻¹, at least 29 s⁻¹, at least 30 s⁻¹, at least 31 s⁻¹, at least 32 s⁻¹, at least 33 s⁻¹, at least 34 s⁻¹, at least 35 s⁻¹, at least 36 s⁻¹, at least 37 s⁻¹, at least 38 s⁻¹, at least 39 s⁻¹, at least 40 s⁻¹, at least 41 s⁻¹, at least 42 s⁻¹, at least 43 s⁻¹, at least 44 s⁻¹, at least 45 s⁻¹, at least 46 s⁻¹, at least 47 s⁻¹, at least 48 s⁻¹, at least 49 s⁻¹, at least 50 s⁻¹, at least 51 s⁻¹, at least 52 s⁻¹, at least 53 s⁻¹, at least 54 s⁻¹, at least 55 s⁻¹, at least 56 s⁻¹, at least 57 s⁻¹, at least 58 s⁻¹, at least 59 s⁻¹, at least 60 s⁻¹, at least 61 s⁻¹, at least 62 s⁻¹, at least 63 s⁻¹, at least 64 s⁻¹, at least 65 s⁻¹, at least 66 s⁻¹, at least 67 s⁻¹, at least 68 s⁻¹, at least 69 s⁻¹, at least 70 s⁻¹, at least 71 s⁻¹, at least 72 s⁻¹, at least 73 s⁻¹, at least 74 s⁻¹, at least 75 s⁻¹, at least 76 s⁻¹, at least 77 s⁻¹, at least 78 s⁻¹, at least 79 s⁻¹, at least 80 s⁻¹, at least 81 s⁻¹, at least 82 s⁻¹, at least 83 s⁻¹, at least 84 s⁻¹, at least 85 s 1, at least 86 s⁻¹, at least 87 s⁻¹, at least 88 s⁻¹, at least 89 s⁻¹, at least 90 s⁻¹, at least 91 s⁻¹, at least 92 s⁻¹, at least 93 s⁻¹, at least 94 s⁻¹, at least 95 s⁻¹, at least 96 s⁻¹, at least 97 s⁻¹, at least 98 s⁻¹, at least 99 s⁻¹, at least 100 s⁻¹, at least 500 s⁻¹, or at least 1000 s⁻¹, In some embodiments, the K_{cat} is between 1 s⁻¹ and 100 s⁻¹, between 5 s⁻¹ and 50 s⁻¹, or between 10 s⁻¹ and 50 s⁻¹.

In some embodiments, the activity of a phosphoketolase described herein is measured as the Michaelis constant (Kₘ). In some embodiments, the Kₘ is less than 0.1 mM, less than 0.2 mM, less than 0.3 mM, less than 0.4 mM, less than 0.5 mM, less than 0.6 mM, less than 0.7 mM, less than 0.8 mM, less than 0.9 mM, less than 1 mM, less than 2 mM, less than 3 mM, less than 4 mM, less than 5 mM, less than 6 mM, less than 7 mM, less than 8 mM, less than 9 mM, less than 10 mM, less than 11 mM, less than 12 mM, less than 13 mM, less than 14 mM, less than 15 mM, less than 16 mM, less than 17 mM, less than 18 mM, less than 19 mM, less than 20 mM, less than 21 mM, less than 22 mM, less than 23 mM, less than 24 mM, less than 25 mM, less than 26 mM, less than 27 mM, less than 28 mM, less than 29 mM, less than 30 mM, less than 31 mM, less than 32 mM, less than 33 mM, less than 34 mM, less than 35 mM, less than 36 mM, less than 37 mM, less than 38 mM, less than 39 mM, less than 40 mM, less than 41 mM, less than 42 mM, less than 43 mM, less than 44 mM, less than 45 mM, less than 46 mM, less than 47 mM, less than 48 mM, less than 49 mM, less than 50 mM, less than 51 mM, less than 52 mM, less than 53 mM, less than 54 mM, less than 55 mM, less than 56 mM, less than 57 mM, less than 58 mM, less than 59 mM, less than 60 mM, less than 61 mM, less than 62 mM, less than 63 mM, less than 64 mM, less than 65 mM, less than 66 mM, less than 67 mM, less than 68 mM, less than 69 mM, less than 70 mM, less than 71 mM, less than 72 mM, less than 73 mM, less than 74 mM, less than 75 mM, less than 76 mM, less than 77 mM, less than 78 mM, less than 79 mM, less than 80 mM, less than 81 mM, less than 82 mM, less than 83 mM, less than 84 mM, less than 85 mM, less than 86 mM, less than 87 mM, less than 88 mM, less than 89 mM, less than 90 mM, less than 91 mM, less than 92 mM, less than 93 mM, less than 94 mM, less than 95 mM, less than 96 mM, less than 97 mM, less than 98 mM, less than 99 mM, less than 100 mM, less than 500 mM, or less than 1000 mM, In some embodiments, the Kₘ is between 0.5 mM and 10 mM, between 1 mM and 10 mM, between 2 mM and 10 mM, between 3 mM and 10 mM, between 4 mM and 10 mM, between 5 mM and 10 mM, between 6 mM and 10 mM, between 7 mM and 10 mM, between 8 mM and 10 mM, or between 9 mM and 10 mM.

In some embodiments, the activity of a phosphoketolase described herein is measured as the catalytic efficiency (k_{cat}/kₘ). In some embodiments, the catalytic efficiency is measured in units of liter/(millimole*second). In some embodiments, the catalytic efficiency is greater than 0.1, greater than 0.2, greater than 0.3, greater than 0.4, greater than 0.5, greater than 0.6, greater than 0.7, greater than 0.8, greater than 0.9, greater than 1, greater than 2, greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 31, greater than 32, greater than 33, greater than 34, greater than 35, greater than 36, greater than 37, greater than 38, greater than 39, greater than 40, greater than 41, greater than 42, greater than 43, greater than 44, greater than 45, greater than 46, greater than 47, greater than 48, greater than 49, greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, greater than 99, greater than 100, greater than 500, greater than 1000. In some embodiments, the catalytic efficiency (k_{cat}/kₘ) is between 1 and 30 liter/(millimole*second), between 5 and 30 liter/(millimole*second), between 1 and 10 liter/(millimole*second), between 10 and 30 liter/(millimole*second), or between 20 and 30 liter/(millimole*second).

In some embodiments, an engineered phosphoketolase provided herein is a variant of a reference polypeptide, wherein the reference polypeptide has an amino acid sequence of SEQ ID NO: 1 or 2, and the engineered phosphoketolase has one or more alterations at a position described in Tables 1, 2, 3 and/or 4 relative to SEQ ID NO: 1 or 2. Accordingly, in some embodiments, an engineered phosphoketolase provided herein is a variant of SEQ ID NO: 1, and has one or more alterations at a position described in Tables 1, 3 and/or 4 relative to SEQ ID NO: 1. In some embodiments, an engineered phosphoketolase provided herein is a variant of SEQ ID NO: 2, and has one or more alterations at a position described in Tables 2, 3 and/or 4 relative to SEQ ID NO: 2.

In some embodiments, an engineered phosphoketolase provided herein has an amino acid sequence that is a variant of SEQ ID NO: 1 or 2 that includes one or more alterations as described in Tables 1, 2, 3, and/or 4, wherein the portion, other than the one or more alterations described in Tables 1, 2, 3, and/or 4, of the engineered phosphoketolase has at least 65%, 70%, 75%, 80%, 85%, 90%, 95%, 98% or 99% sequence identity, or is identical, to an amino acid sequence referenced as SEQ ID NO:1 or 2. Accordingly, in some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 65% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 70% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 75% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 80% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 85% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 90% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 95% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 98% identical to SEQ ID NO: 1. In some embodiments, an engineered phosphoketolase provided herein has an amino acid sequence that includes one or more alterations as described in Tables 1, 3, and/or 4 and the portion, other than the alteration described in Tables 1, 3, and/or 4, of the engineered phosphoketolase has at least 99% identical to SEQ ID NO: 1. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 65% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 70% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 75% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 80% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 85% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 90% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 95% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 98% identical to SEQ ID NO:2. In some embodiments, an engineered phosphoketolase provided herein has an amino acid sequence that includes one or more alterations as described in Tables 2, 3, and/or 4 and the portion, other than the alteration described in Tables 2, 3, and/or 4, of the engineered phosphoketolase has at least 99% identical to SEQ ID NO:2.

Sequence identity, homology or similarity refers to sequence similarity between two polypeptides or between two nucleic acid molecules. Identity can be determined by comparing a position in each sequence which may be aligned for purposes of comparison. When a position in the compared sequence is occupied by the same base or amino acid, then the molecules are identical at that position. A degree of homology between sequences is a function of the number of matching or homologous positions shared by the sequences. A polypeptide or polypeptide region (or a polynucleotide or polynucleotide region) has a certain percentage (for example, 65%, 70%, 75%, 80%, 85%, 90%, 95%, 98% or 99%) of “sequence identity” to another sequence means that, when aligned, that percentage of amino acids (or nucleotide bases) are the same in comparing the two sequences. The alignment of two sequences to determine their percent sequence identity can be done using software programs known in the art, such as, for example, those described in Ausubel et al., Current Protocols in Molecular Biology, John Wiley and Sons, Baltimore, MD (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information (see also Altschul et al., “J. Mol. Biol. 215:403-410 (1990)).

In some embodiments, an engineered phosphoketolase provided herein includes one or more amino acid substitutions at a residue corresponding to position 49, 60, 63, 64, 65, 77, 104, 136, 156, 158, 174, 200, 208, 219, 220, 221, 239, 257, 267, 276, 305, 323, 335, 364, 365, 372, 377, 407, 420, 436, 438, 440, 442, 447, 478, 483, 499, 504, 505, 542, 547, 548, 549, 567, 572, 584, 593, 611, 621, 627, 641, 687, 759, 765, 767, 781, 786, 788, 799, and/or 804 in SEQ ID NO: 1.

In some embodiments, an engineered phosphoketolase provided herein includes one or more amino acid substitutions at a residue corresponding to position 49, 60, 63, 77, 104, 136, 174, 200, 208, 221, 239, 257, 267, 276, 305, 323, 364, 365, 372, 377, 420, 442, 447, 483, 499, 504, 548, 567, 572, 584, 593, 611, 621, 627, 641, 687, 759, 765, 781, 786, 788, 799, and/or 804 in SEQ ID NO: 1.

In some embodiments, an engineered phosphoketolase provided herein includes one or more amino acid substitutions at a residue corresponding to position 63, 64, 65, 136, 156, 158, 219, 220, 221, 267, 276, 305, 335, 364, 372, 407, 420, 436, 438, 440, 442, 478, 504, 505, 542, 547, 548, 549, 572, 584, 593, 627, 641, 687, 767, and/or 781 in SEQ ID NO: 1.

In some embodiments, an engineered phosphoketolase provided herein includes one or more amino acid substitutions at a residue corresponding to position 63, 136, 221, 267, 276, 305, 364, 372, 420, 504, 572, 584, 593, 627, 641, 687, and/or 781 in SEQ ID NO: 1.

In some embodiments, an engineered phosphoketolase provided herein includes one or more amino acid substitutions at a residue corresponding to position 60, 64, 104, 136, 158, 174, 221, 267, 335, 365, 407, 436, 447, 499, 548, 572, 593, 621, 687, 765, 767, 781, 786, and/or 788 in SEQ ID NO: 1.

In some embodiments, an engineered phosphoketolase provided herein includes one or more amino acid substitutions at a residue corresponding to position 63, 221, 323, 442, 499, 504, and/or 548 in SEQ ID NO: 1.

In some embodiments, an engineered phosphoketolase provided herein includes one or more amino acid substitutions at a residue corresponding to position 63, 64, 65, 156, 158, 219, 220, 221, 436, 438, 440, 442, 478, 504, 505, 542, 547, 548, and/or 549 in SEQ ID NO: 1.

In some embodiments, an engineered phosphoketolase provided herein includes one or more amino acid substitutions at a residue corresponding to position 43, 69, 73, 113, 145, 167, 183, 230, 248, 266, 276, 306, 336, 337, 349, 379, 420, 449, 451, 460, 513, 518, 560, 562, 581, 585, 606, 614, 620, 634, 700, 773, 779, 781, 795, 800, 802, and/or 813 in SEQ ID NO: 2.

In some embodiments, an engineered phosphoketolase provided herein includes one or more amino acid substitutions at a residue corresponding to position 43, 349, 420, and/or 795 in SEQ ID NO: 2.

In some embodiments, an engineered phosphoketolase provided herein includes one or more amino acid substitutions at a residue corresponding to position 43, 69, 73, 113, 145, 167, 183, 230, 276, 306, 336, 349, 379, 420, 449, 451, 460, 513, 560, 562, 585, 606, 614, 620, 634, 700, 779, 781, 795, 800, and/or 802 in SEQ ID NO: 2.

In some embodiments, an engineered phosphoketolase provided herein includes one or more amino acid substitutions at a residue corresponding to position 69, 73, 113, 145, 167, 183, 230, 276, 349, 379, 420, 449, 460, 513, 562, 585, 606, 634, 700, 779, 795, 800, and/or 802 in SEQ ID NO: 2.

In some embodiments, an engineered phosphoketolase provided herein includes one or more amino acid substitutions at a residue corresponding to position 73, 167, 230, 336, 449, 451, 513, 560, 562, and/or 620 in SEQ ID NO: 2.

In some embodiments, an engineered phosphoketolase provided herein includes one or more alterations at a position described in Tables 1, 2, 3, and/or 4, wherein the one or more amino acid alterations are conservative amino acid substitutions. In some embodiments, an engineered phosphoketolase provided herein includes one or more conservative amino acid substitutions relative to an alteration described in Tables 1, 2, 3, and/or 4. As a non-limiting example, a conservative amino acid substitution relative to the M49L substitution in SEQ ID NO: 1 may include substitution of M49 for another non-polar (hydrophobic) amino acid (e.g., Cys (C), Ala (A), Val (V), Ile (I), Pro (P), Phe (F), Met (M), Trp (W), Gly (G), or Tyr (Y)). In some embodiments, an engineered phosphoketolase provided herein includes one or more alterations at a position described in Tables 1, 2, 3, and/or 4, wherein the one or more amino acid alterations are non-conservative amino acid substitutions. In some embodiments, an engineered phosphoketolase provided herein includes one or more alterations at a position described in Table 1. In some embodiments, an engineered phosphoketolase provided herein includes one or more alterations at a position described in Table 2. In some embodiments, an engineered phosphoketolase provided herein includes one or more alterations at a position described in Table 3. In some embodiments, an engineered phosphoketolase provided herein includes one or more alterations at a position described in Table 4. In some embodiments, an engineered phosphoketolase provided herein includes a conservative amino acid substitution and/or non-conservative amino acid substitution in 1 to 10 amino acid positions as set forth in Tables 1, 2, 3, and/or 4.

In some embodiments, an engineered phosphoketolase provided herein can further include a conservative amino acid substitution in from 1 to 50 amino acid positions, or alternatively from 2 to 50 amino acid positions, or alternatively from 3 to 50 amino acid positions, or alternatively from 4 to 50 amino acid positions, or alternatively from 5 to 50 amino acid positions, or alternatively from 6 to 50 amino acid positions, or alternatively from 7 to 50 amino acid positions, or alternatively from 8 to 50 amino acid positions, or alternatively from 9 to 50 amino acid positions, or alternatively from 10 to 50 amino acid positions, or alternatively from 15 to 50 amino acid positions, or alternatively from 20 to 50 amino acid positions, or alternatively from 30 to 50 amino acid positions, or alternatively from 40 to 50 amino acid positions, or alternatively from 45 to 50 amino acid positions, or any integer therein, wherein the positions are other than the variant amino acid positions set forth in Tables 1, 2, 3 and/or 4. In some aspects, such a conservative amino acid sequence is a chemically conservative or an evolutionary conservative amino acid substitution. Methods of identifying conservative amino acids are well known to one of skill in the art, any one of which can be used to generate the isolated polypeptides described herein.

An engineered phosphoketolase provided herein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 alterations relative to a wild-type or parent phosphoketolase. An engineered phosphoketolase provided herein may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 alterations relative to a wild-type or parent phosphoketolase. The one or more alterations may be located at one or more positions corresponding to the one or more positions described in Tables 1-4. The one or more alterations may be located at one or more positions corresponding to one or more positions in SEQ ID NO: 1 and/or SEQ ID NO: 2. As used herein, the phrase “a residue corresponding to position X in SEQ ID NO: Y” refers to a residue at a corresponding position following an alignment of two sequences. For example, the residue in SEQ ID NO: 1 corresponding to position 49 in SEQ ID NO: 2 is the residue at position 47 in SEQ ID NO: 1. In some embodiments, a reference sequence is a phosphoketolase that is not SEQ ID NO: 1 or SEQ ID NO: 2.

An engineered phosphoketolase provided herein can include any combination of the alterations set forth in Tables 1, 2, 3, and/or 4. One alteration alone, or in combination, can produce an engineered phosphoketolase that retains or improves the activity as described herein relative to a reference polypeptide, for example, the wild-type (native) phosphoketolase. In some embodiments, an engineered phosphoketolase provided herein includes at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 alterations as set forth in Tables 1, 2, 3, and/or 4, including up to an alteration at all of the positions identified in Tables 1, 2, 3, and/or 4. In some embodiments, an engineered phosphoketolase provided herein includes at least 2 alterations as set forth in Tables 1, 2, 3, and/or 4. In some embodiments, an engineered phosphoketolase provided herein includes at least 3 alterations as set forth in Tables 1, 2, 3, and/or 4. In some embodiments, an engineered phosphoketolase provided herein includes at least 4 alterations as set forth in Tables 1, 2, 3, and/or 4. In some embodiments, an engineered phosphoketolase provided herein includes at least 5 alterations as set forth in Tables 1, 2, 3, and/or 4. In some embodiments, an engineered phosphoketolase provided herein includes at least 6 alterations as set forth in Tables 1, 2, 3, and/or 4. In some embodiments, an engineered phosphoketolase provided herein includes at least 7 alterations as set forth in Tables 1, 2, 3, and/or 4. In some embodiments, an engineered phosphoketolase provided herein includes at least 8 alterations as set forth in Tables 1, 2, 3, and/or 4. In some embodiments, an engineered phosphoketolase provided herein includes at least 9 alterations as set forth in Tables 1, 2, 3, and/or 4. In some embodiments, an engineered phosphoketolase provided herein includes at least 10 alterations as set forth in Tables 1, 2, 3, and/or 4.

In some embodiments, the one or more amino acid alterations of the engineered phosphoketolase is an alteration described in Table 1. For example, in some embodiments, the one or more amino acid alternations result in an engineered phosphoketolase comprising: a) L at a residue corresponding to position 49 in SEQ ID NO: 1; b) P at a residue corresponding to position 60 in SEQ ID NO: 1; c) L at a residue corresponding to position 63 in SEQ ID NO: 1; d) A or S at a residue corresponding to position 64 in SEQ ID NO: 1; e) Y at a residue corresponding to position 65 in SEQ ID NO: 1; f) A at a residue corresponding to position 77 in SEQ ID NO: 1; g) L or V at a residue corresponding to position 104 in SEQ ID NO: 1; h) F at a residue corresponding to position 136 in SEQ ID NO: 1; i) A at a residue corresponding to position 156 in SEQ ID NO: 1; j) I, V, M, or A at a residue corresponding to position 158 in SEQ ID NO: 1; k) D at a residue corresponding to position 174 in SEQ ID NO:1; l) F at a residue corresponding to position 200 in SEQ ID NO: 1; m) A at a residue corresponding to position 208 in SEQ ID NO: 1; n) C, N, D, S, M, or E at a residue corresponding to position 219 in SEQ ID NO: 1; o) M at a residue corresponding to position 220 in SEQ ID NO: 1; p) Q, C, T, V, R, N, M, H, E, or G at a residue corresponding to position 221 in SEQ ID NO: 1; q) R at a residue corresponding to position 239 in SEQ ID NO: 1; r) P at a residue corresponding to position 257 in SEQ ID NO: 1; s) A at a residue corresponding to position 267 in SEQ ID NO: 1; t) R or K at a residue corresponding to position 276 in SEQ ID NO: 1; u) G at a residue corresponding to position 305 in SEQ ID NO: 1; v) I at a residue corresponding to position 323 in SEQ ID NO: 1; w) L at a residue corresponding to position 335 in SEQ ID NO: 1; x) L at a residue corresponding to position 364 in SEQ ID NO: 1; y) A at a residue corresponding to position 365 in SEQ ID NO: 1; z) M at a residue corresponding to position 372 in SEQ ID NO: 1; aa) H at a residue corresponding to position 377 in SEQ ID NO: 1; bb) R at a residue corresponding to position 407 in SEQ ID NO: 1; cc) L at a residue corresponding to position 420 in SEQ ID NO: 1; dd) A, V, S, C, or E at a residue corresponding to position 436 in SEQ ID NO: 1; ee) D or T at a residue corresponding to position 438 in SEQ ID NO: 1; ff) I or K at a residue corresponding to position 440 in SEQ ID NO: 1; gg) Q, A, D, T, H, M, V, C, S, or I at a residue corresponding to position 442 in SEQ ID NO: 1; hh) V at a residue corresponding to position 447 in SEQ ID NO: 1; ii) I or M at a residue corresponding to position 478 in SEQ ID NO: 1; j) C at a residue corresponding to position 483 in SEQ ID NO: 1; kk) F at a residue corresponding to position 499 in SEQ ID NO: 1; ll) A at a residue corresponding to position 504 in SEQ ID NO: 1; mm) M at a residue corresponding to position 505 in SEQ ID NO: 1; nn) A, G, W, or I at a residue corresponding to position 542 in SEQ ID NO:1; oo) E or A at a residue corresponding to position 547 in SEQ ID NO: 1; pp) T, I, E, V, M, N, P, or G at a residue corresponding to position 548 in SEQ ID NO: 1; qq) Y at a residue corresponding to position 549 in SEQ ID NO: 1; rr) S at a residue corresponding to position 567 in SEQ ID NO: 1; ss) D or E at a residue corresponding to position 572 in SEQ ID NO: 1; tt) T, S, or C at a residue corresponding to position 584 in SEQ ID NO: 1; uu) L at a residue corresponding to position 593 in SEQ ID NO: 1; vv) Q at a residue corresponding to position 611 in SEQ ID NO: 1; ww) H at a residue corresponding to position 621 in SEQ ID NO: 1; xx) G at a residue corresponding to position 627 in SEQ ID NO: 1; yy) P at a residue corresponding to position 641 in SEQ ID NO: 1; zz) H at a residue corresponding to position 687 in SEQ ID NO: 1; aaa) L at a residue corresponding to position 759 in SEQ ID NO: 1; bbb) R at a residue corresponding to position 765 in SEQ ID NO: 1; ccc) P at a residue corresponding to position 767 in SEQ ID NO: 1; ddd) Q, K, or R at a residue corresponding to position 781 in SEQ ID NO: 1; eee) K or R at a residue corresponding to position 786 in SEQ ID NO: 1; fff) Y at a residue corresponding to position 788 in SEQ ID NO: 1; ggg) I at a residue corresponding to position 799 in SEQ ID NO: 1; and/or hhh) W at a residue corresponding to position 804 in SEQ ID NO: 1. In some embodiments, the one or more amino acid alterantions result in an engeneered phosphoketolase comprising: a) L at a residue corresponding to position 49 in SEQ ID NO: 1; b) P at a residue corresponding to position 60 in SEQ ID NO: 1; c) L at a residue corresponding to position 63 in SEQ ID NO: 1; d) A at a residue corresponding to position 77 in SEQ ID NO: 1; e) L or V at a residue corresponding to position 104 in SEQ ID NO: 1; f) F at a residue corresponding to position 136 in SEQ ID NO: 1; g) D at a residue corresponding to position 174 in SEQ ID NO: 1; h) F at a residue corresponding to position 200 in SEQ ID NO: 1; i) A at a residue corresponding to position 208 in SEQ ID NO: 1; j) Q, C, N, or T at a residue corresponding to position 221 in SEQ ID NO: 1; k) R at a residue corresponding to position 239 in SEQ ID NO: 1; l) P at a residue corresponding to position 257 in SEQ ID NO: 1; m) A at a residue corresponding to position 267 in SEQ ID NO: 1; n) R at a residue corresponding to position 276 in SEQ ID NO: 1; o) G at a residue corresponding to position 305 in SEQ ID NO: 1; p) I at a residue corresponding to position 323 in SEQ ID NO: 1; q) L at a residue corresponding to position 364 in SEQ ID NO: 1; r) A at a residue corresponding to position 365 in SEQ ID NO: 1; s) M at a residue corresponding to position 372 in SEQ ID NO: 1; t) H at a residue corresponding to position 377 in SEQ ID NO: 1; u) L at a residue corresponding to position 420 in SEQ ID NO: 1; v) Q at a residue corresponding to position 442 in SEQ ID NO: 1; w) V at a residue corresponding to position 447 in SEQ ID NO: 1; x) F at a residue corresponding to position 499 in SEQ ID NO: 1; y) C at a residue corresponding to position 483 in SEQ ID NO: 1; z) A at a residue corresponding to position 504 in SEQ ID NO: 1; aa) T at a residue corresponding to position 548 in SEQ ID NO: 1; bb) S at a residue corresponding to position 567 in SEQ ID NO: 1; cc) D or E at a residue corresponding to position 572 in SEQ ID NO: 1; dd) T, S, or C at a residue corresponding to position 584 in SEQ ID NO: 1; ee) L at a residue corresponding to position 593 in SEQ ID NO: 1; ff) Q at a residue corresponding to position 611 in SEQ ID NO: 1; gg) H at a residue corresponding to position 621 in SEQ ID NO: 1; hh) G at a residue corresponding to position 627 in SEQ ID NO: 1; ii) P at a residue corresponding to position 641 in SEQ ID NO: 1; jj) H at a residue corresponding to position 687 in SEQ ID NO: 1; kk) L at a residue corresponding to position 759 in SEQ ID NO: 1; 11) R at a residue corresponding to position 765 in SEQ ID NO: 1; mm) Q or K at a residue corresponding to position 781 in SEQ ID NO: 1; nn) K or R at a residue corresponding to position 786 in SEQ ID NO: 1; oo) Y at a residue corresponding to position 788 in SEQ ID NO: 1; pp) I at a residue corresponding to position 799 in SEQ ID NO: 1; and/or qq) W at a residue corresponding to position 804 in SEQ ID NO: 1. In some embodiments, the one or more amino acid alternations result in an engineered phosphoketolase comprising: a) L at a residue corresponding to position 63 in SEQ ID NO: 1; b) A or S at a residue corresponding to position 64 in SEQ ID NO: 1; c) Y at a residue corresponding to position 65 in SEQ ID NO: 1; d) F at a residue corresponding to position 136 in SEQ ID NO: 1; e) A at a residue corresponding to position 156 in SEQ ID NO: 1; f) 1, V, M, or A at a residue corresponding to position 158 in SEQ ID NO: 1; g) C, N, D, S, M, or E at a residue corresponding to position 219 in SEQ ID NO: 1; h) M at a residue corresponding to position 220 in SEQ ID NO: 1; i) V, R, N, M, H, E, G, C, or T at a residue corresponding to position 221 in SEQ ID NO: 1; j) A at a residue corresponding to position 267 in SEQ ID NO: 1; k) K, R at a residue corresponding to position 276 in SEQ ID NO:1; l) G at a residue corresponding to position 305 in SEQ ID NO: 1; m) L at a residue corresponding to position 335 in SEQ ID NO: 1; n) L at a residue corresponding to position 364 in SEQ ID NO: 1; o) M at a residue corresponding to position 372 in SEQ ID NO: 1; p) R at a residue corresponding to position 407 in SEQ ID NO: 1; q) L at a residue corresponding to position 420 in SEQ ID NO: 1; r) A, V, S, C, or E at a residue corresponding to position 436 in SEQ ID NO: 1; s) D, T at a residue corresponding to position 438 in SEQ ID NO: 1; t) I, K at a residue corresponding to position 440 in SEQ ID NO: 1; u) A, D, T, H, M, V, C, S, or I at a residue corresponding to position 442 in SEQ ID NO: 1; v) I, M at a residue corresponding to position 478 in SEQ ID NO: 1; w) A at a residue corresponding to position 504 in SEQ ID NO:1; x) M at a residue corresponding to position 505 in SEQ ID NO: 1; y) A, G, W, or I at a residue corresponding to position 542 in SEQ ID NO: 1; z) E, A at a residue corresponding to position 547 in SEQ ID NO: 1; aa) T, I, E, V, M, N, P, or G, at a residue corresponding to position 548 in SEQ ID NO: 1; bb) Y at a residue corresponding to position 549 in SEQ ID NO: 1; cc) E at a residue corresponding to position 572 in SEQ ID NO: 1; dd) S or T at a residue corresponding to position 584 in SEQ ID NO: 1; ee) L at a residue corresponding to position 593 in SEQ ID NO:1; ff) G at a residue corresponding to position 627 in SEQ ID NO: 1; gg) P at a residue corresponding to position 641 in SEQ ID NO: 1; hh) H at a residue corresponding to position 687 in SEQ ID NO: 1; ii) P at a residue corresponding to position 767 in SEQ ID NO: 1; and/or jj) R, K, or Q at a residue corresponding to position 781 in SEQ ID NO: 1. In some embodiments, the one or more amino acid alternations result in an engineered phosphoketolase comprising: a) L at a residue corresponding to position 63 in SEQ ID NO: 1; b) F at a residue corresponding to position 136 in SEQ ID NO: 1; c) C or T at a residue corresponding to position 221 in SEQ ID NO: 1; d) A at a residue corresponding to position 267 in SEQ ID NO: 1; e) R at a residue corresponding to position 276 in SEQ ID NO: 1; f) G at a residue corresponding to position 305 in SEQ ID NO: 1; g) L at a residue corresponding to position 364 in SEQ ID NO: 1; h) M at a residue corresponding to position 372 in SEQ ID NO: 1; i) L at a residue corresponding to position 420 in SEQ ID NO: 1; j) A at a residue corresponding to position 504 in SEQ ID NO: 1; k) D or E at a residue corresponding to position 572 in SEQ ID NO: 1; l) T, S, or C at a residue corresponding to position 584 in SEQ ID NO: 1; m) L at a residue corresponding to position 593 in SEQ ID NO: 1; n) G at a residue corresponding to position 627 in SEQ ID NO: 1; o) P at a residue corresponding to position 641 in SEQ ID NO: 1; p) H at a residue corresponding to position 687 in SEQ ID NO: 1; and/or q) Q or K at a residue corresponding to position 781 in SEQ ID NO: 1. In some embodiments, the one or more amino acid alternations result in an engineered phosphoketolase comprising: a) P at a residue corresponding to position 60 in SEQ ID NO: 1; b) A at a residue corresponding to position 64 in SEQ ID NO: 1; c) V or L at a residue corresponding to position 104 in SEQ ID NO: 1; d) F at a residue corresponding to position 136 in SEQ ID NO: 1; e) I at a residue corresponding to position 158 in SEQ ID NO: 1; f) D at a residue corresponding to position 174 in SEQ ID NO: 1; g) Q or H at a residue corresponding to position 221 in SEQ ID NO: 1; h) A at a residue corresponding to position 267 in SEQ ID NO: 1; i) L at a residue corresponding to position 335 in SEQ ID NO: 1; j) A at a residue corresponding to position 365 in SEQ ID NO: 1; k) R at a residue corresponding to position 407 in SEQ ID NO: 1; l) A at a residue corresponding to position 436 in SEQ ID NO: 1; m) V at a residue corresponding to position 447 in SEQ ID NO: 1; n) F at a residue corresponding to position 499 in SEQ ID NO: 1; o) G, N, T, or I at a residue corresponding to position 548 in SEQ ID NO: 1; p) D at a residue corresponding to position 572 in SEQ ID NO: 1; q) L at a residue corresponding to position 593 in SEQ ID NO: 1; r) H at a residue corresponding to position 621 in SEQ ID NO: 1; s) H at a residue corresponding to position 687 in SEQ ID NO: 1; t) R at a residue corresponding to position 765 in SEQ ID NO: 1; u) P at a residue corresponding to position 767 in SEQ ID NO: 1; v) R or Q at a residue corresponding to position 781 in SEQ ID NO: 1; w) K or R at a residue corresponding to position 786 in SEQ ID NO: 1; and/or x) Y at a residue corresponding to position 788 in SEQ ID NO: 1. In some embodiments, the one or more amino acid alternations result in an engineered phosphoketolase comprising: a) L at a residue corresponding to position 63 in SEQ ID NO: 1; b) C, T, N, or Q at a residue corresponding to position 221 in SEQ ID NO: 1; c) I at a residue corresponding to position 323 in SEQ ID NO: 1; d) Q at a residue corresponding to position 442 in SEQ ID NO: 1; e) F at a residue corresponding to position 499 in SEQ ID NO: 1; f) A at a residue corresponding to position 504 in SEQ ID NO: 1; and/or g) T at a residue corresponding to position 548 in SEQ ID NO: 1. In some embodiments, the one or more amino acid alternations result in an engineered phosphoketolase comprising: a) L at position 63 in SEQ ID NO: 1; b) A or S at position 64 in SEQ ID NO: 1; c) Y at position 65 in SEQ ID NO: 1; d) Y at position 156 in SEQ ID NO: 1; e) I, V, M, or A at position 158 in SEQ ID NO: 1; f) C, N, D, S, M, or E at position 219 in SEQ ID NO: 1; g) M at position 220 in SEQ ID NO: 1; h) V, R, N, M, H, E, G, C, or T at position 221 in SEQ ID NO: 1; i) A, V, S, C, or E at position 436 in SEQ ID NO: 1; j) D or T at position 438 in SEQ ID NO: 1; k) I or K at position 440 in SEQ ID NO: 1; l) A, D, T, H, M, V, C, S, or I at position 442 in SEQ ID NO: 1; m) I or M at position 478 in SEQ ID NO: 1; n) A at position 504 in SEQ ID NO: 1; o) M at position 505 in SEQ ID NO: 1; p) A, G, W, or I at position 542 in SEQ ID NO: 1; q) E or A at position 547 in SEQ ID NO: 1; r) T, I, E, V, M, N, P, or G at position 548 in SEQ ID NO: 1; and/or s) Y at position 549 in SEQ ID NO: 1.

In some embodiments, the one or more amino acid alterations of the engineered phosphoketolase is an alteration described in Table 2. For example, in some embodiments, the one or more amino acid alternations result in an engineered phosphoketolase comprising: a) D at a residue corresponding to position 43 in SEQ ID NO: 2; b) P at a residue corresponding to position 69 in SEQ ID NO: 2; c) A at a residue corresponding to position 73 in SEQ ID NO: 2; d) V or L at a residue corresponding to position 113 in SEQ ID NO: 2; e) F at a residue corresponding to position 145 in SEQ ID NO: 2; f) I at a residue corresponding to position 167 in SEQ ID NO: 2; g) D at a residue corresponding to position 183 in SEQ ID NO: 2; h) N, Q or H at a residue corresponding to position 230 in SEQ ID NO: 2; i) R at a residue corresponding to position 248 in SEQ ID NO: 2; j) P at a residue corresponding to position 266 in SEQ ID NO: 2; k) A at a residue corresponding to position 276 in SEQ ID NO: 2; l) W at a residue corresponding to position 306 in SEQ ID NO: 2; m) S at a residue corresponding to position 336 in SEQ ID NO: 2; n) I at a residue corresponding to position 337 in SEQ ID NO: 2; o) L at a residue corresponding to position 349 in SEQ ID NO: 2; p) A at a residue corresponding to position 379 in SEQ ID NO: 2; q) R at a residue corresponding to position 420 in SEQ ID NO: 2; r) A at a residue corresponding to position 449 in SEQ ID NO: 2; s) I at a residue corresponding to position 451 in SEQ ID NO: 2; t) V at a residue corresponding to position 460 in SEQ ID NO: 2; u) F at a residue corresponding to position 513 in SEQ ID NO: 2; v) A at a residue corresponding to position 518 in SEQ ID NO: 2; w) K, L, T, M, Y, Q, G, or E at a residue corresponding to position 560 in SEQ ID NO: 2; x) H, G, F, W, C, N, T, or I at a residue corresponding to position 562 in SEQ ID NO: 2; y) S at a residue corresponding to position 581 in SEQ ID NO: 2; z) D at a residue corresponding to position 585 in SEQ ID NO: 2; aa) L at a residue corresponding to position 606 in SEQ ID NO: 2; bb) V at a residue corresponding to position 614 in SEQ ID NO: 2; cc) K at a residue corresponding to position 620 in SEQ ID NO: 2; dd) H at a residue corresponding to position 634 in SEQ ID NO: 2; ee) H at a residue corresponding to position 700 in SEQ ID NO: 2; ff) L at a residue corresponding to position 773 in SEQ ID NO: 2; gg) R at a residue corresponding to position 779 in SEQ ID NO: 2; hh) P at a residue corresponding to position 781 in SEQ ID NO: 2; ii) R or Q at a residue corresponding to position 795 in SEQ ID NO: 2; j) K or R at a residue corresponding to position 800 in SEQ ID NO: 2; kk) Y at a residue corresponding to position 802 in SEQ ID NO: 2; and/or ll) I at a residue corresponding to position 813 in SEQ ID NO: 2. The engineered phosphoketolase of claim 27, wherein the one or more amino acid alternations result in an engineered phosphoketolase comprising: a) D at a residue corresponding to position 43 in SEQ ID NO: 2; b) L at a residue corresponding to position 349 in SEQ ID NO: 2; c) R at a residue corresponding to position 420 in SEQ ID NO: 2; and/or d) R at a residue corresponding to position 795 in SEQ ID ON: 2. The engineered phosphoketolase of claim 27, wherein the one or more amino acid alternations result in an engineered phosphoketolase comprising: a) D at a residue corresponding to position 43 in SEQ ID NO: 2; b) P at a residue corresponding to position 69 in SEQ ID NO: 2; c) A at a residue corresponding to position 73 in SEQ ID NO: 2; d) V or L at a residue corresponding to position 113 in SEQ ID NO: 2; e) F at a residue corresponding to position 145 in SEQ ID NO: 2; f) 1 at a residue corresponding to position 167 in SEQ ID NO: 2; g) D at a residue corresponding to position 183 in SEQ ID NO: 2; h) Q or H at a residue corresponding to position 230 in SEQ ID NO: 2; i) A at a residue corresponding to position 276 in SEQ ID NO: 2; j) W at a residue corresponding to position 306 in SEQ ID NO: 2; k) S at a residue corresponding to position 336 in SEQ ID NO: 2; l) L at a residue corresponding to position 349 in SEQ ID NO: 2; m) A at a residue corresponding to position 379 in SEQ ID NO: 2; n) R at a residue corresponding to position 420 in SEQ ID NO: 2; o) A at a residue corresponding to position 449 in SEQ ID NO: 2; p) I at a residue corresponding to position 451 in SEQ ID NO: 2; q) V at a residue corresponding to position 460 in SEQ ID NO: 2; r) F at a residue corresponding to position 513 in SEQ ID NO: 2; s) K, L, T, M, Y, Q, G, or E at a residue corresponding to position 560 in SEQ ID NO: 2; t) H, G, F, W, C, N, T, or I at a residue corresponding to position 562 in SEQ ID NO: 2; u) D at a residue corresponding to position 585 in SEQ ID NO: 2; v) L at a residue corresponding to position 606 in SEQ ID NO: 2; w) V at a residue corresponding to position 614 in SEQ ID NO: 2; x) K at a residue corresponding to position 620 in SEQ ID NO: 2; y) H at a residue corresponding to position 634 in SEQ ID NO: 2; z) H at a residue corresponding to position 700 in SEQ ID NO: 2; aa) R at a residue corresponding to position 779 in SEQ ID NO: 2; bb) P at a residue corresponding to position 781 in SEQ ID NO: 2; cc) R or Q at a residue corresponding to position 795 in SEQ ID NO: 2; dd) K or R at a residue corresponding to position 800 in SEQ ID NO: 2; and/or ee) Y at a residue corresponding to position 802 in SEQ ID NO: 2. The engineered phosphoketolase of claim 27, wherein the one or more amino acid alternations result in an engineered phosphoketolase comprising: a) P at a residue corresponding to position 69 in SEQ ID NO: 2; b) A at a residue corresponding to position 73 in SEQ ID NO: 2; c) L or V at a residue corresponding to position 113 in SEQ ID NO: 2; d) F at a residue corresponding to position 145 in SEQ ID NO: 2; e) I at a residue corresponding to position 167 in SEQ ID NO: 2; f) D at a residue corresponding to position 183 in SEQ ID NO: 2; g) Q or H at a residue corresponding to position 230 in SEQ ID NO: 2; h) A at a residue corresponding to position 276 in SEQ ID NO: 2; i) L at a residue corresponding to position 349 in SEQ ID NO: 2; j) A at a residue corresponding to position 379 in SEQ ID NO: 2; k) R at a residue corresponding to position 420 in SEQ ID NO: 2; l) A at a residue corresponding to position 449 in SEQ ID NO: 2; m) V at a residue corresponding to position 460 in SEQ ID NO: 2; n) F at a residue corresponding to position 513 in SEQ ID NO: 2; o) T, N, I, or G at a residue corresponding to position 562 in SEQ ID NO: 2; p) D at a residue corresponding to position 585 in SEQ ID NO: 2; q) L at a residue corresponding to position 606 in SEQ ID NO: 2; r) H at a residue corresponding to position 634 in SEQ ID NO: 2; s) H at a residue corresponding to position 700 in SEQ ID NO: 2; t) R at a residue corresponding to position 779 in SEQ ID NO: 2; u) Q or R at a residue corresponding to position 795 in SEQ ID NO: 2; v) K or R at a residue corresponding to position 800 in SEQ ID NO: 2; and/or w) Y at a residue corresponding to position 802 in SEQ ID NO: 2. In some embodiments, the one or more amino acid alternations result in an engineered phosphoketolase comprising one or more amino acid alternations result in an engineered phosphoketolase comprising: a) A at a residue corresponding to position 73 in SEQ ID NO: 2; b) I at a residue corresponding to position 167 in SEQ ID NO: 2; c) Q or H at a residue corresponding to position 230 in SEQ ID NO: 2; d) S at a residue corresponding to position 336 in SEQ ID NO: 2; e) A at a residue corresponding to position 449 in SEQ ID NO: 2; f) I at a residue corresponding to position 451 in SEQ ID NO: 2; g) F at a residue corresponding to position 513 in SEQ ID NO: 2; h) K, L, T, M, Y, Q, G, or E at a residue corresponding to position 560 in SEQ ID NO: 2; i) H, G, F, W, C, N, T, or I at a residue corresponding to position 562 in SEQ ID NO: 2; and/or j) K at a residue corresponding to position 620 in SEQ ID NO: 2.

Methods of generating and assaying the engineered phosphoketolases described herein are well known to one of skill in the art. Examples of such methods are described in Example 1. Any of a variety of methods can be used to generate an engineered phosphoketolase disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, M D (1999); Gillman et al., Directed Evolution Library Creation: Methods and Protocols (Methods in Molecular Biology) Springer, 2nd ed (2014)). One non-limiting example of a method for preparing an engineered phosphoketolase is to express recombinant nucleic acids encoding the engineered phosphoketolase in a suitable microbial organism, such as a bacterial cell, a yeast cell, or other suitable cell, using methods well known in the art.

In some embodiments, an engineered phosphoketolase provided herein is an isolated phosphoketolase. An isolated engineered phosphoketolases provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, the provided herein is a recombinant nucleic acid that has a nucleotide sequence encoding an engineered phosphoketolase described herein. Accordingly, in some embodiments, provided herein is a recombinant nucleic acid selected from (a) a nucleic acid molecule encoding an engineered phosphoketolase comprising a variant of amino acid sequence SEQ ID NO:1 or 2, wherein the engineered phosphoketolase comprises one or more alterations at a position described in Tables 1, 2, 3, and/or 4; (b) a recombinant nucleic acid that hybridizes to an isolated nucleic acid of (a) under highly stringent hybridization conditions; and (c) a recombinant nucleic acid that is complementary to (a) or (b).

In some embodiments, provided herein is a recombinant nucleic acid encoding an engineered phosphoketolase comprising a variant of amino acid sequence SEQ ID NO:1 or 2, wherein the engineered phosphoketolase comprises one or more alterations at a position described in Tables 1, 2, 3, and/or 4. In some embodiments, the recombinant nucleic acid encodes an engineered phosphoketolase comprising one or more alterations at a position described in Table 1. In some embodiments, the recombinant nucleic acid encodes an engineered phosphoketolase comprising one or more alterations at a position described in Table 2. In some embodiments, the recombinant nucleic acid encodes an engineered phosphoketolase comprising one or more alterations at a position described in Table 3. In some embodiments, the recombinant nucleic acid encodes an engineered phosphoketolase comprising one or more alterations at a position described in Table 4.

In some embodiments, provided herein is a recombinant nucleic acid that hybridizes under highly stringent hybridization conditions to a isolated nucleic acid encoding an engineered phosphoketolase described herein. Accordingly, in some embodiments, the recombinant nucleic acid is an isolated nucleic acid that hybridizes under highly stringent hybridization conditions to a nucleic acid that encodes an engineered phosphoketolase comprising one or more alterations at a position described in Table 1. In some embodiments, the recombinant nucleic acid molecule is an isolated nucleic acid that hybridizes under highly stringent hybridization conditions to a nucleic acid that encodes an engineered phosphoketolase comprising one or more alterations at a position described in Table 2. In some embodiments, the recombinant nucleic acid is an isolated nucleic acid that hybridizes under highly stringent hybridization conditions to a nucleic acid that encodes an engineered phosphoketolase comprising one or more alterations at a position described in Table 3. In some embodiments, the recombinant nucleic acid is an isolated nucleic acid that hybridizes under highly stringent hybridization conditions to a recombinant nucleic acid that encodes an engineered phosphoketolase comprising one or more alterations at a position described in Table 4.

In some embodiments, provided herein is a recombinant nucleic acid encoding an engineered phosphoketolase comprising an amino acid sequence that is a variant of SEQ ID NO: 1 or 2 that includes one or more alterations as described in Tables 1, 2, 3, and/or 4, wherein the portion, other than the alteration described in Tables 1, 2, 3, and/or 4, of the engineered phosphoketolase has at least 65%, 70%, 75%, 80%, 85%, 90%, 95%, 98% or 99% sequence identity, or is identical, to an amino acids sequence referenced as SEQ ID NO:1 or 2. Accordingly, in some embodiments, a recombinant nucleic acid encodes an engineered phosphoketolase comprising an amino acid sequence that includes one or more alterations as described in Tables 1, 3, and/or 4 and the portion, other than the alteration described in Tables 1, 3, and/or 4, of the engineered phosphoketolase has at least 65% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 70% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 75% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 80% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 85% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 90% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 95% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 98% identical to SEQ ID NO: 1. In some embodiments, a recombinant nucleic acid encodes an engineered phosphoketolase that has an amino acid sequence that includes one or more alterations as described in Tables 1, 3, and/or 4 and the portion, other than the alteration described in Tables 1, 3, and/or 4, of the engineered phosphoketolase has at least 99% identical to SEQ ID NO: 1. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 65% identical to SEQ ID NO:2. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 70% identical to SEQ ID NO:2. In some embodiments, a recombinant nucleic acid molecule encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 75% identical to SEQ ID NO:2. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 80% identical to SEQ ID NO:2. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 85% identical to SEQ ID NO:2. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 90% identical to SEQ ID NO:2. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 95% identical to SEQ ID NO:2. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 98% identical to SEQ ID NO:2. In some embodiments, a recombinant nucleic acid encodes an engineered phosphoketolase that has an amino acid sequence that includes one or more alterations as described in Tables 2, 3, and/or 4 and the portion, other than the alteration described in Tables 2, 3, and/or 4, of the engineered phosphoketolase has at least 99% identical to SEQ ID NO:2.

In some embodiments, a recombinant nucleic acid encodes an engineered phosphoketolase that includes one or more alterations at a position described in Tables 1, 2, 3, and/or 4, wherein the one or more amino acid alterations are conservative amino acid substitutions. In some embodiments, a recombinant nucleic acid encodes an engineered phosphoketolase that includes one or more alterations at a position described in Tables 1, 2, 3, and/or 4, wherein the one or more amino acid alterations are non-conservative amino acid substitutions. In some embodiments, a recombinant nucleic acid encodes an engineered phosphoketolase that includes one or more alterations at a position described in Table 1. In some embodiments, a recombinant nucleic acid encodes an engineered phosphoketolase that includes one or more alterations at a position described in Table 2. In some embodiments, a recombinant nucleic acid encodes an engineered phosphoketolase that includes one or more alterations at a position described in Table 3. In some embodiments, a recombinant nucleic acid encodes an engineered phosphoketolase that includes one or more alterations at a position described in Table 4. In some embodiments, a recombinant nucleic acid encodes an engineered phosphoketolase that includes a conservative amino acid substitution and/or non-conservative amino acid substitution in 1 to 10 amino acid positions as set forth in Tables 1, 2, 3, and/or 4.

In some embodiments, a recombinant nucleic acid encodes an engineered phosphoketolase that includes a conservative amino acid substitution in from 1 to 50 amino acid positions, or alternatively from 2 to 50 amino acid positions, or alternatively from 3 to 50 amino acid positions, or alternatively from 4 to 50 amino acid positions, or alternatively from 5 to 50 amino acid positions, or alternatively from 6 to 50 amino acid positions, or alternatively from 7 to 50 amino acid positions, or alternatively from 8 to 50 amino acid positions, or alternatively from 9 to 50 amino acid positions, or alternatively from 10 to 50 amino acid positions, or alternatively from 15 to 50 amino acid positions, or alternatively from 20 to 50 amino acid positions, or alternatively from 30 to 50 amino acid positions, or alternatively from 40 to 50 amino acid positions, or alternatively from 45 to 50 amino acid positions, or any integer therein, wherein the positions are other than the variant amino acid positions set forth in Tables 1, 2, 3 and/or 4. In some aspects, such a conservative amino acid sequence is a chemically conservative or an evolutionary conservative amino acid substitution. Methods of identifying conservative amino acids are well known to one of skill in the art, any one of which can be used to generate the isolated polypeptides described herein.

A recombinant nucleic acid provided herein can encode an engineered phosphoketolase that include any combination of the alterations set forth in Tables 1, 2, 3, and/or 4. One alteration alone, or in combination, can produce an engineered phosphoketolase that retains or improves the activity as described herein relative to a reference polypeptide, for example, the wild-type (native) phosphoketolase. In some embodiments, a recombinant nucleic acid encodes an engineered phosphoketolase that includes at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 alterations as set forth in Tables 1, 2, 3, and/or 4, including up to an alteration at all of the positions identified in Tables 1, 2, 3, and/or 4. In some embodiments, a recombinant nucleic acid encodes an engineered phosphoketolase that includes at least 2 alterations as set forth in Tables 1, 2, 3, and/or 4. In some embodiments, a recombinant nucleic acid encodes an engineered phosphoketolase that includes at least 3 alterations as set forth in Tables 1, 2, 3, and/or 4. In some embodiments, a recombinant nucleic acid encodes an engineered phosphoketolase that includes at least 4 alterations as set forth in Tables 1, 2, 3, and/or 4. In some embodiments, a recombinant nucleic acid encodes an engineered phosphoketolase that includes at least 5 alterations as set forth in Tables 1, 2, 3, and/or 4. In some embodiments, a recombinant nucleic acid encodes an engineered phosphoketolase that includes at least 6 alterations as set forth in Tables 1, 2, 3, and/or 4. In some embodiments, a recombinant nucleic acid encodes an engineered phosphoketolase that includes at least 7 alterations as set forth in Tables 1, 2, 3, and/or 4. In some embodiments, a recombinant nucleic acid encodes an engineered phosphoketolase that includes at least 8 alterations as set forth in Tables 1, 2, 3, and/or 4. In some embodiments, a recombinant nucleic acid encodes an engineered phosphoketolase that includes at least 9 alterations as set forth in Tables 1, 2, 3, and/or 4. In some embodiments, a recombinant nucleic acid encodes an engineered phosphoketolase that includes at least 10 alterations as set forth in Tables 1, 2, 3, and/or 4.

In some embodiments, provided herein is a recombinant nucleic acid that includes a nucleotide sequence encoding an engineered phosphoketolase described herein that is operatively linked to a promoter. Such a promoter can express the engineered phosphoketolase in a microbial organism as described herein.

In some embodiments, provided herein is a vector containing a recombinant nucleic acid described herein. In some embodiments, the vector is an expression vector. In some embodiments, the vector comprises double stranded DNA.

A recombinant nucleic acid encoding an engineered phosphoketolase described herein also includes a nucleic acid that hybridizes to a nucleic acid disclosed herein or a nucleic acid that hybridizes to a nucleic acid that encodes an amino acid sequence disclosed. Hybridization conditions can include highly stringent, moderately stringent, or low stringency hybridization conditions that are well known to one of skill in the art such as those described herein. Similarly, a recombinant nucleic acid that can be used in the compositions and methods described herein can be described as having a certain percent sequence identity to a nucleic acid disclosed herein or a nucleic acid that hybridizes to a nucleic acid molecule that encodes an amino acid sequence disclosed herein. For example, the nucleic acid can have at least 65%, 70%, 75%, 80%, 85%, 90%, 91%, 92%, 93%, 94%, 95%, 96%, 97%, 98% or 99% sequence identity, or be identical, to a nucleotide described herein.

Stringent hybridization refers to conditions under which hybridized polynucleotides are stable. As known to those of skill in the art, the stability of hybridized polynucleotides is reflected in the melting temperature (Tₘ) of the hybrids. In general, the stability of hybridized polynucleotides is a function of the salt concentration, for example, the sodium ion concentration, and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleotide sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C. Hybridization conditions other than highly stringent hybridization conditions can also be used to describe the nucleotide sequences disclosed herein. For example, the phrase moderately stringent hybridization refers to conditions equivalent to hybridization in 50% formamide, 5× Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 10% formamide, 5× Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamine tetraacetic acid (EDTA)) contains 3M sodium chloride, 0.2M sodium phosphate, and 0.025 M (EDTA). Other suitable low, moderate and high stringency hybridization buffers and conditions are well known to those of skill in the art and are described, for example, in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered phosphoketolase described herein can have at least a certain sequence identity to a nucleotide sequence disclosed herein. Accordingly, in some aspects described herein, a recombinant nucleic acid encoding an engineered phosphoketolase has a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid disclosed herein or a nucleic acid that hybridizes to a nucleic acid that encodes an amino acid sequence disclosed herein.

It is understood that a recombinant nucleic acid described herein or an engineered phosphoketolase described here can exclude a wild-type parental sequence, for example a parental sequence, such as SEQ ID NOS: 1 or 2. One skilled in the art will readily understand the meaning of a parental wild-type sequence based on what is well known in the art. It is further understood that such a recombinant nucleic acid described herein can exclude a nucleotide sequence encoding a naturally occurring amino acid sequence as found in nature. Similarly, an engineered phosphoketolase described herein can exclude an amino acid sequence as found in nature. Thus, in some embodiments, the recombinant nucleic acid or engineered phosphoketolase described herein is as set forth herein, with the proviso that the encoded amino acid sequence is not the wild-type parental sequence or a naturally occurring amino acid sequence and/or that the nucleotide sequence is not a wild-type or naturally occurring nucleotide sequence. A naturally occurring amino acid or nucleotide sequence is understood by those skilled in the art as relating to a sequence that is found in a naturally occurring organism as found in nature. Thus, a nucleotide or amino acid sequence that is not found in the same state or having the same nucleotide or encoded amino acid sequence as in a naturally occurring organism is included within the meaning of a recombinant nucleotide and/or amino acid sequence described herein. For example, a nucleotide or amino acid sequence that has been altered at one or more nucleotide or amino acid positions from a parent sequence, including variants as described herein, are included within the meaning of a nucleotide or amino acid sequence described herein that is not naturally occurring. A recombinant nucleic acid described herein excludes a naturally occurring chromosome that contains the nucleotide sequence, and can further exclude other molecules, as found in a naturally occurring cell, such as DNA binding proteins, for example, proteins such as histones that bind to chromosomes within a eukaryotic cell.

Thus, a recombinant nucleic acid described here has physical and chemical differences compared to a naturally occurring nucleic acid. A recombinant or non-naturally occurring nucleic acid described herein does not contain or does not necessarily have some or all of the chemical bonds, either covalent or non-covalent bonds, of a naturally occurring nucleic acid as found in nature. A recombinant nucleic acid described herein thus differs from a naturally occurring nucleic acid, for example, by having a different chemical structure than a naturally occurring nucleic acid as found in a chromosome. A different chemical structure can occur, for example, by cleavage of phosphodiester bonds that release a recombinant nucleic acid from a naturally occurring chromosome. A recombinant nucleic acid described herein can also differ from a naturally occurring nucleic acid by isolating or separating the nucleic acid from proteins that bind to chromosomal DNA in either prokaryotic or eukaryotic cells, thereby differing from a naturally occurring nucleic acid by different non-covalent bonds. With respect to nucleic acids of prokaryotic origin, a non-naturally occurring nucleic acid described herein does not necessarily have some or all of the naturally occurring chemical bonds of a chromosome, for example, binding to DNA binding proteins such as polymerases or chromosome structural proteins, or is not in a higher order structure such as being supercoiled. With respect to nucleic acids of eukaryotic origin, a non-naturally occurring nucleic acid described herein also does not contain the same internal nucleic acid chemical bonds or chemical bonds with structural proteins as found in chromatin. For example, a non-naturally occurring nucleic acid described herein is not chemically bonded to histones or scaffold proteins and is not contained in a centromere or telomere. Thus, the non-naturally occurring nucleic acids described herein are chemically distinct from a naturally occurring nucleic acid because they either lack or contain different van der Waals interactions, hydrogen bonds, ionic or electrostatic bonds, and/or covalent bonds from a nucleic acid as found in nature. Such differences in bonds can occur either internally within separate regions of the nucleic acid (that is cis) or such difference in bonds can occur in trans, for example, interactions with chromosomal proteins. In the case of a nucleic acid of eukaryotic origin, a cDNA is considered to be a recombinant or non-naturally occurring nucleic acid since the chemical bonds within a cDNA differ from the covalent bonds, that is the sequence, of a gene on chromosomal DNA. Thus, it is understood by those skilled in the art that recombinant or non-naturally occurring nucleic acid is distinct from a naturally occurring nucleic acid.

In some embodiments, provided herein is a method of constructing a host strain that can include, among other steps, introducing a vector disclosed herein into a microbial organism, for example, that is capable of expressing an amino acid sequence encoded by the vector and/or is capable of fermentation. Vectors described herein can be introduced stably or transiently into a microbial organism using techniques well known in the art including, but not limited to, conjugation, electroporation, chemical transformation, transduction, transfection, and ultrasound transformation. Additional methods are disclosed herein, any one of which can be used in the method described herein.

In some embodiments, provided herein is a microbial organism, in particular a non-naturally occurring microbial organism, that comprises a polypeptide described herein, that is, an engineered phosphoketolase described herein. Thus, provided herein is a non-naturally occurring microbial organism having a recombinant nucleic acid encoding an engineered phosphoketolase described herein. Accordingly, in some embodiments, provided herein is microbial organism (e.g., host microbial organism) that comprises a recombinant polynucleotide encoding an engineered phosphoketolase, wherein the engineered phosphoketolase comprises one or more amino acid alterations at a residue corresponding to position 49, 60, 63, 64, 65, 77, 104, 136, 156, 158, 174, 200, 208, 219, 220, 221, 239, 257, 267, 276, 305, 323, 335, 364, 365, 372, 377, 407, 420, 436, 438, 440, 442, 447, 478, 483, 499, 504, 505, 542, 547, 548, 549, 567, 572, 584, 593, 611, 621, 627, 641, 687, 759, 765, 767, 781, 786, 788, 799, and/or 804 in SEQ ID NO: 1. In some embodiments, provided herein is microbial organism (e.g., host microbial organism) that comprises a recombinant polynucleotide encoding an engineered phosphoketolase, wherein the engineered phosphoketolase comprises one or more amino acid alterations at a residue corresponding to position 43, 69, 73, 113, 145, 167, 183, 230, 248, 266, 276, 306, 336, 337, 349, 379, 420, 449, 451, 460, 513, 518, 560, 562, 581, 585, 606, 614, 620, 634, 700, 773, 779, 781, 795, 800, 802, and/or 813 in SEQ ID NO: 2.

Optionally, the non-naturally occurring microbial organism can include one or more exogenous nucleic acids encoding one or more enzymes for production of acetyl-CoA. Accordingly, in some embodiments, the non-naturally occurring microbial organism can include an exogenous nucleic acid encoding (a) an acetate kinase and an acetyl-CoA transferase, synthetase, or ligase, wherein the acetate kinase catalyzes the conversion of acetyl-phosphate to acetate and the acetyl-CoA transferase, synthetase, or ligase catalyzes the conversion of acetate to acetyl-CoA; (b) a phosphotransacetylase, wherein the phosphotransacetylase catalyzes the conversion of acetyl-phosphate to acetyl-CoA; (c) a pyruvate dehydrogenase, a pyruvate ferredoxin oxidoreductase, or a pyruvate:NADP+ oxidoreductase, wherein the pyruvate dehydrogenase, the pyruvate ferredoxin oxidoreductase, or the pyruvate:NADP+ oxidoreductase catalyzes the conversion of pyruvate to acetyl-CoA and carbon dioxide; or (d) a pyruvate formate lyase, wherein the pyruvate formate lyase catalyzes the conversion of pyruvate to acetyl-CoA and formate. In some embodiments, the non-naturally occurring microbial organism provided herein includes one or more exogenous nucleic acids encoding an acetate kinase and an acetyl-CoA transferase, synthetase, or ligase, wherein the acetate kinase catalyzes the conversion of acetyl-phosphate to acetate and the acetyl-CoA transferase, synthetase, or ligase catalyzes the conversion of acetate to acetyl-CoA. In some embodiments, the non-naturally occurring microbial organism provided herein includes an exogenous nucleic acid encoding a phosphotransacetylase, wherein the phosphotransacetylase catalyzes the conversion of acetyl-phosphate to acetyl-CoA. In some embodiments, the non-naturally occurring microbial organism provided herein includes one or more exogenous nucleic acids encoding (c) a pyruvate dehydrogenase, a pyruvate ferredoxin oxidoreductase, or a pyruvate:NADP+ oxidoreductase, wherein the pyruvate dehydrogenase, the pyruvate ferredoxin oxidoreductase, or the pyruvate:NADP+ oxidoreductase catalyzes the conversion of pyruvate to acetyl-CoA and carbon dioxide. In some embodiments, the non-naturally occurring microbial organism provided herein includes an exogenous nucleic acid encoding a pyruvate formate lyase, wherein the pyruvate formate lyase catalyzes the conversion of pyruvate to acetyl-CoA and formate. Exemplary genes and gene products (e.g., enzymes or proteins) that can be used to introduce the pathways described above are well known in the art (see, e.g., U.S. Patent Publication 2017/0159075, the disclosure of which is incorporated herein by reference). In some embodiments, the exogenous nucleic acid is heterologous. In some embodiments, the exogenous nucleic acid is homologous.

Optionally, the non-naturally occurring microbial organism can also include an exogenous nucleic acid encoding one or more enzymes for production of pyruvate. Accordingly, in some embodiments, the non-naturally occurring microbial organism can include one or more exogenous nucleic acids encoding a combination of enzymes that catalyze the conversion of glyceraldehyde-3-phosphate to pyruvate. Such a combination of enzymes can include a glyceraldehyde-3-phosphate dehydrogenase, a phosphoglycerate kinase, a phosphoglyceromutase, an enolase, a pyruvate kinase and a PTS-dependent substrate import. Exemplary genes and gene products (e.g., enzymes or proteins) that can be used to for production ofpyruvate are well known in the art (see, e.g., U.S. Patent Publication 2017/0159075, the disclosure of which is incorporated herein by reference). In some embodiments, the exogenous nucleic acid is heterologous. In some embodiments, the exogenous nucleic acid is homologous.

In some embodiments, provided herein is a non-naturally occurring microbial organism as described herein that further includes a pathway capable of producing a bioderived compound as described herein. In some aspects, the bioderived compound is an alcohol, a glycol, an organic acid, an alkene, a diene, an organic amine, an organic aldehyde, a vitamin, a nutraceutical or a pharmaceutical.

In some embodiments, the non-naturally occurring microbial organism described herein includes a pathway for production of an alcohol as described herein. Accordingly, in some embodiments, the alcohol is selected from: (i) a biofuel alcohol, wherein said biofuel is a primary alcohol, a secondary alcohol, a diol or triol comprising C3 to C10 carbon atoms; (ii) n-propanol or isopropanol; and (iii) a fatty alcohol, wherein said fatty alcohol comprises C4 to C27 carbon atoms, C8 to C18 carbon atoms, C12 to C18 carbon atoms, or C12 to C14 carbon atoms. In some aspects, the biofuel alcohol is selected from 1-propanol, isopropanol, 1-butanol, isobutanol, 1-pentanol, isopentenol, 2-methyl-1-butanol, 3-methyl-1-butanol, 1-hexanol, 3-methyl-1-pentanol, 1-heptanol, 4-methyl-1-hexanol, and 5-methyl-1-hexanol.

In some embodiments, the non-naturally occurring microbial organism described herein includes a pathway for production of an diol. Accordingly, in some embodiments, the diol is a propanediol or a butanediol. In some aspects, the butanediol is 1,4 butanediol, 1,3-butanediol or 2,3-butanediol.

In some embodiments, the non-naturally occurring microbial organism described herein includes a pathway for production of a bioderived compound selected from: (i) 1,4-butanediol or an intermediate thereto, wherein said intermediate is optionally 4-hydroxybutanoic acid (4-HB); (ii) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol; (iii) 1,3-butanediol or an intermediate thereto, wherein said intermediate is optionally 3-hydroxybutyrate (3-HB), 2,4-pentadienoate, crotyl alcohol or 3-buten-1-ol; (iv) adipate, 6-aminocaproic acid, caprolactam, hexamethylenediamine, levulinic acid or an intermediate thereto, wherein said intermediate is optionally adipyl-CoA or 4-aminobutyryl-CoA; (v) methacrylic acid or an ester thereof, 3-hydroxyisobutyrate, 2-hydroxyisobutyrate, or an intermediate thereto, wherein said ester is optionally methyl methacrylate or poly(methyl methacrylate); (vi) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto; (vii) succinic acid or an intermediate thereto; and (viii) a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol). Accordingly, in some embodiments, the non-naturally occurring microbial organism described herein includes a pathway for production of 1,4-butanediol or an intermediate thereto, wherein said intermediate is optionally 4-hydroxybutanoic acid (4-HB). In some embodiments, the non-naturally occurring microbial organism described herein includes a pathway for production of butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol. In some embodiments, the non-naturally occurring microbial organism described herein includes a pathway for production of 1,3-butanediol or an intermediate thereto, wherein said intermediate is optionally 3-hydroxybutyrate (3-HB), 2,4-pentadienoate, crotyl alcohol or 3-buten-1-ol. In some embodiments, the non-naturally occurring microbial organism described herein includes a pathway for production of adipate, 6-aminocaproic acid, caprolactam, hexamethylenediamine, levulinic acid or an intermediate thereto, wherein said intermediate is optionally adipyl-CoA or 4-aminobutyryl-CoA. In some embodiments, the non-naturally occurring microbial organism described herein includes a pathway for production of methacrylic acid or an ester thereof, 3-hydroxyisobutyrate, 2-hydroxyisobutyrate, or an intermediate thereto, wherein said ester is optionally methyl methacrylate or poly(methyl methacrylate). In some embodiments, the non-naturally occurring microbial organism described herein includes a pathway for production of 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto. In some embodiments, the non-naturally occurring microbial organism described herein includes a pathway for production of succinic acid or an intermediate thereto. In some embodiments, the non-naturally occurring microbial organism described herein includes a pathway for production of a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

1,4-Butanediol and intermediates thereto, such as 4-hydroxybutanoic acid (4-hydroxybutanoate, 4-hydroxybutyrate, 4-HB), are bioderived compounds that can be made via enzymatic pathways described herein and in the following publications. Suitable bioderived compound pathways and enzymes, methods for screening and methods for isolating are found in: WO2008115840A2 published 25 Sep. 2008 entitled Compositions and Methods for the Biosynthesis of 1,4-Butanediol and Its Precursors; WO2010141780A1 published 9 Dec. 2010 entitled Process of Separating Components of A Fermentation Broth; WO2010141920A2 published 9 Dec. 2010 entitled Microorganisms for the Production of 1,4-Butanediol and Related Methods; WO2010030711A2 published 18 Mar. 2010 entitled Microorganisms for the Production of 1,4-Butanediol; WO2010071697A1 published 24 Jun. 2010 Microorganisms and Methods for Conversion of Syngas and Other Carbon Sources to Useful Products; WO2009094485A1 published 30 Jul. 2009 Methods and Organisms for Utilizing Synthesis Gas or Other Gaseous Carbon Sources and Methanol; WO2009023493A1 published 19 Feb. 2009 entitled Methods and Organisms for the Growth-Coupled Production of 1,4-Butanediol; and WO2008115840A2 published 25 Sep. 2008 entitled Compositions and Methods for the Biosynthesis of 1,4-Butanediol and Its Precursors, which are all incorporated herein by reference.

Butadiene and intermediates thereto, such as 1,4-butanediol, 2,3-butanediol, 1,3-butanediol, crotyl alcohol, 3-buten-2-ol (methyl vinyl carbinol) and 3-buten-1-ol, are bioderived compounds that can be made via enzymatic pathways described herein and in the following publications. In addition to direct fermentation to produce butadiene, 1,3-butanediol, 1,4-butanediol, crotyl alcohol, 3-buten-2-ol (methyl vinyl carbinol) or 3-buten-1-ol can be separated, purified (for any use), and then chemically dehydrated to butadiene by metal-based catalysis. Suitable bioderived compound pathways and enzymes, methods for screening and methods for isolating are found in: WO2011140171A2 published 10 Nov. 2011 entitled Microorganisms and Methods for the Biosynthesis of Butadiene; WO2012018624A2 published 9 Feb. 2012 entitled Microorganisms and Methods for the Biosynthesis of Aromatics, 2,4-Pentadienoate and 1,3-Butadiene; WO2011140171A2 published 10 Nov. 2011 entitled Microorganisms and Methods for the Biosynthesis of Butadiene; WO2013040383A1 published 21 Mar. 2013 entitled Microorganisms and Methods for Producing Alkenes; WO2012177710A1 published 27 Dec. 2012 entitled Microorganisms for Producing Butadiene and Methods Related thereto; WO2012106516A1 published 9 Aug. 2012 entitled Microorganisms and Methods for the Biosynthesis of Butadiene; and WO2013028519A1 published 28 Feb. 2013 entitled Microorganisms and Methods for Producing 2,4-Pentadienoate, Butadiene, Propylene, 1,3-Butanediol and Related Alcohols, which are all incorporated herein by reference.

1,3-Butanediol and intermediates thereto, such as 2,4-pentadienoate, crotyl alcohol or 3-buten-1-ol, are bioderived compounds that can be made via enzymatic pathways described herein and in the following publications. Suitable bioderived compound pathways and enzymes, methods for screening and methods for isolating are found in: WO2011071682A1 published 16 Jun. 2011 entitled Methods and Organisms for Converting Synthesis Gas or Other Gaseous Carbon Sources and Methanol to 1,3-Butanediol; WO2011031897 Å published 17 Mar. 2011 entitled Microorganisms and Methods for the Co-Production of Isopropanol with Primary Alcohols, Diols and Acids; WO2010127319A2 published 4 Nov. 2010 entitled Organisms for the Production of 1,3-Butanediol; WO2013071226A1 published 16 May 2013 entitled Eukaryotic Organisms and Methods for Increasing the Availability of Cytosolic Acetyl-CoA, and for Producing 1,3-Butanediol; WO2013028519A1 published 28 Feb. 2013 entitled Microorganisms and Methods for Producing 2,4-Pentadienoate, Butadiene, Propylene, 1,3-Butanediol and Related Alcohols; WO2013036764A1 published 14 Mar. 2013 entitled Eukaryotic Organisms and Methods for Producing 1,3-Butanediol; WO2013012975A1 published 24 Jan. 2013 entitled Methods for Increasing Product Yields; and WO2012177619A2 published 27 Dec. 2012 entitled Microorganisms for Producing 1,3-Butanediol and Methods Related Thereto, which are all incorporated herein by reference.

Adipate, 6-aminocaproic acid, caprolactam, hexamethylenediamine and levulinic acid, and their intermediates, e.g. 4-aminobutyryl-CoA, are bioderived compounds that can be made via enzymatic pathways described herein and in the following publications. Suitable bioderived compound pathways and enzymes, methods for screening and methods for isolating are found in: WO2010129936A1 published 11 Nov. 2010 entitled Microorganisms and Methods for the Biosynthesis of Adipate, Hexamethylenediamine and 6-Aminocaproic Acid; WO2013012975A1 published 24 Jan. 2013 entitled Methods for Increasing Product Yields; WO2012177721A1 published 27 Dec. 2012 entitled Microorganisms for Producing 6-Aminocaproic Acid; WO2012099621A1 published 26 Jul. 2012 entitled Methods for Increasing Product Yields; and WO2009151728 published 17 Dec. 2009 entitled Microorganisms for the production of adipic acid and other compounds, which are all incorporated herein by reference.

Methacrylic acid (2-methyl-2-propenoic acid) is used in the preparation of its esters, known collectively as methacrylates (e.g. methyl methacrylate, which is used most notably in the manufacture of polymers). Methacrylate esters such as methyl methacrylate, 3-hydroxyisobutyrate and/or 2-hydroxyisobutyrate and their intermediates are bioderived compounds that can be made via enzymatic pathways described herein and in the following publications. Suitable bioderived compound pathways and enzymes, methods for screening and methods for isolating are found in: WO2012135789A2 published 4 Oct. 2012 entitled Microorganisms for Producing Methacrylic Acid and Methacrylate Esters and Methods Related Thereto; and WO2009135074A2 published 5 Nov. 2009 entitled Microorganisms for the Production of Methacrylic Acid, which are all incorporated herein by reference.

1,2-Propanediol (propylene glycol), n-propanol, 1,3-propanediol and glycerol, and their intermediates are bioderived compounds that can be made via enzymatic pathways described herein and in the following publications. Suitable bioderived compound pathways and enzymes, methods for screening and methods for isolating are found in: WO2009111672A1 published 9 Nov. 2009 entitled Primary Alcohol Producing Organisms; WO2011031897A1 17 Mar. 2011 entitled Microorganisms and Methods for the Co-Production of Isopropanol with Primary Alcohols, Diols and Acids; WO2012177599A2 published 27 Dec. 2012 entitled Microorganisms for Producing N-Propanol 1,3-Propanediol, 1,2-Propanediol or Glycerol and Methods Related Thereto, which are all incorporated herein by referenced.

Succinic acid and intermediates thereto, which are useful to produce products including polymers (e.g. PBS), 1,4-butanediol, tetrahydrofuran, pyrrolidone, solvents, paints, deicers, plastics, fuel additives, fabrics, carpets, pigments, and detergents, are bioderived compounds that can be made via enzymatic pathways described herein and in the following publication. Suitable bioderived compound pathways and enzymes, methods for screening and methods for isolating are found in: EP1937821A2 published 2 Jul. 2008 entitled Methods and Organisms for the Growth-Coupled Production of Succinate, which is incorporated herein by reference.

Primary alcohols and fatty alcohols (also known as long chain alcohols), including fatty acids and fatty aldehydes thereof, and intermediates thereto, are bioderived compounds that can be made via enzymatic pathways in the following publications. Suitable bioderived compound pathways and enzymes, methods for screening and methods for isolating are found in: WO2009111672 published 11 Sep. 2009 entitled Primary Alcohol Producing Organisms; WO2012177726 published 27 Dec. 2012 entitled Microorganism for Producing Primary Alcohols and Related Compounds and Methods Related Thereto, which are all incorporated herein by reference.

Further suitable bioderived compounds that the microbial organisms described herein can be used to produce via acetyl-CoA, including optionally further through acetoacetyl-CoA and/or succinyl-CoA, are included as part of the present disclosure. Exemplary well known bioderived compounds, their pathways and enzymes for production, methods for screening and methods for isolating are found in the following patents and publications: succinate (U.S. publication 2007/0111294, WO 2007/030830, WO 2013/003432), 3-hydroxypropionic acid (3-hydroxypropionate) (U.S. publication 2008/0199926, WO 2008/091627, U.S. publication 2010/0021978), 1,4-butanediol (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. Pat. No. 8,129,169, WO 2010/141920, U.S. publication 2011/0201068, WO 2011/031897, U.S. Pat. No. 8,377,666, WO 2011/047101, U.S. publication 2011/0217742, WO 2011/066076, U.S. publication 2013/0034884, WO 2012/177943), 4-hydroxybutanoic acid (4-hydroxybutanoate, 4-hydroxybutyrate, 4-hydroxybutryate) (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. Pat. No. 8,129,155, WO 2010/071697), γ-butyrolactone (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. publication 2011/0217742, WO 2011/066076), 4-hydroxybutyryl-CoA (U.S. publication 2011/0003355, WO 2010/141780, U.S. publication 2013/0034884, WO 2012/177943), 4-hydroxybutanal (U.S. publication 2011/0003355, WO 2010/141780, U.S. publication 2013/0034884, WO 2012/177943), putrescine (U.S. publication 2011/0003355, WO 2010/141780, U.S. publication 2013/0034884, WO 2012/177943), Olefins (such as acrylic acid and acrylate ester) (U.S. Pat. No. 8,026,386, WO 2009/045637), acetyl-CoA (U.S. Pat. No. 8,323,950, WO 2009/094485), methyl tetrahydrofolate (U.S. Pat. No. 8,323,950, WO 2009/094485), ethanol (U.S. Pat. No. 8,129,155, WO 2010/071697), isopropanol (U.S. Pat. No. 8,129,155, WO 2010/071697, U.S. publication 2010/0323418, WO 2010/127303, U.S. publication 2011/0201068, WO 2011/031897), n-butanol (U.S. Pat. No. 8,129,155, WO 2010/071697), isobutanol (U.S. Pat. No. 8,129,155, WO 2010/071697), n-propanol (U.S. publication 2011/0201068, WO 2011/031897), methylacrylic acid (methylacrylate) (U.S. publication 2011/0201068, WO 2011/031897), primary alcohol (U.S. Pat. No. 7,977,084, WO 2009/111672, WO 2012/177726), long chain alcohol (U.S. Pat. No. 7,977,084, WO 2009/111672, WO 2012/177726), adipate (adipic acid) (U.S. Pat. No. 8,062,871, WO 2009/151728, U.S. Pat. No. 8,377,680, WO 2010/129936, WO 2012/177721), 6-aminocaproate (6-aminocaproic acid) (U.S. Pat. No. 8,062,871, WO 2009/151728, U.S. Pat. No. 8,377,680, WO 2010/129936, WO 2012/177721), caprolactam (U.S. Pat. No. 8,062,871, WO 2009/151728, U.S. Pat. No. 8,377,680, WO 2010/129936, WO 2012/177721), hexamethylenediamine (U.S. Pat. No. 8,377,680, WO 2010/129936, WO 2012/177721), levulinic acid (U.S. Pat. No. 8,377,680, WO 2010/129936), 2-hydroxyisobutyric acid (2-hydroxyisobutyrate) (U.S. Pat. No. 8,241,877, WO 2009/135074, U.S. publication 2013/0065279, WO 2012/135789), 3-hydroxyisobutyric acid (3-hydroxyisobutyrate) (U.S. Pat. No. 8,241,877, WO 2009/135074, U.S. publication 2013/0065279, WO 2012/135789), methacrylic acid (methacrylate) (U.S. Pat. No. 8,241,877, WO 2009/135074, U.S. publication 2013/0065279, WO 2012/135789), methacrylate ester (U.S. publication 2013/0065279, WO 2012/135789), fumarate (fumaric acid) (U.S. Pat. No. 8,129,154, WO 2009/155382), malate (malic acid) (U.S. Pat. No. 8,129,154, WO 2009/155382), acrylate (carboxylic acid) (U.S. Pat. No. 8,129,154, WO 2009/155382), methyl ethyl ketone (U.S. publication 2010/0184173, WO 2010/057022, U.S. Pat. No. 8,420,375, WO 2010/144746), 2-butanol (U.S. publication 2010/0184173, WO 2010/057022, U.S. Pat. No. 8,420,375, WO 2010/144746), 1,3-butanediol (U.S. publication 2010/0330635, WO 2010/127319, U.S. publication 2011/0201068, WO 2011/031897, U.S. Pat. No. 8,268,607, WO 2011/071682, U.S. publication 2013/0109064, WO 2013/028519, U.S. publication 2013/0066035, WO 2013/036764), cyclohexanone (U.S. publication 2011/0014668, WO 2010/132845), terephthalate (terephthalic acid) (U.S. publication 2011/0124911, WO 2011/017560, U.S. publication 2011/0207185, WO 2011/094131, U.S. publication 2012/0021478, WO 2012/018624), muconate (muconic acid) (U.S. publication 2011/0124911, WO 2011/017560), aniline (U.S. publication 2011/0097767, WO 2011/050326), p-toluate (p-toluic acid) (U.S. publication 2011/0207185, WO 2011/094131, U.S. publication 2012/0021478, WO 2012/018624), (2-hydroxy-3-methyl-4-oxobutoxy)phosphonate (U.S. publication 2011/0207185, WO 2011/094131, U.S. publication 2012/0021478, WO 2012/018624), ethylene glycol (U.S. publication 2011/0312049, WO 2011/130378, WO 2012/177983), propylene (U.S. publication 2011/0269204, WO 2011/137198, U.S. publication 2012/0329119, U.S. publication 2013/0109064, WO 2013/028519), butadiene (1,3-butadiene) (U.S. publication 2011/0300597, WO 2011/140171, U.S. publication 2012/0021478, WO 2012/018624, U.S. publication 2012/0225466, WO 2012/106516, U.S. publication 2013/0011891, WO 2012/177710, U.S. publication 2013/0109064, WO 2013/028519), toluene (U.S. publication 2012/0021478, WO 2012/018624), benzene (U.S. publication 2012/0021478, WO 2012/018624), (2-hydroxy-4-oxobutoxy)phosphonate (U.S. publication 2012/0021478, WO 2012/018624), benzoate (benzoic acid) (U.S. publication 2012/0021478, WO 2012/018624), styrene (U.S. publication 2012/0021478, WO 2012/018624), 2,4-pentadienoate (U.S. publication 2012/0021478, WO 2012/018624, U.S. publication 2013/0109064, WO 2013/028519), 3-butene-1-ol (U.S. publication 2012/0021478, WO 2012/018624, U.S. publication 2013/0109064, WO 2013/028519), 3-buten-2-ol (U.S. publication 2013/0109064, WO 2013/028519), 1,4-cyclohexanedimethanol (U.S. publication 2012/0156740, WO 2012/082978), crotyl alcohol (U.S. publication 2013/0011891, WO 2012/177710, U.S. publication 2013/0109064, WO 2013/028519), alkene (U.S. publication 2013/0122563, WO 2013/040383, US 2011/0196180), hydroxyacid (WO 2012/109176), ketoacid (WO 2012/109176), wax esters (WO 2007/136762) or caprolactone (U.S. publication 2013/0144029, WO 2013/067432) pathway. The patents and patent application publications listed above that disclose bioderived compound pathways are herein incorporated herein by reference.

One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, in some embodiments, provided herein is a non-naturally occurring microbial organism containing at least one recombinant nucleic acid encoding an engineered phosphoketolase, where the phosphoketolase functions in a pathway to produce a bioderived compound.

In some embodiments, provided herein is a non-naturally occurring microbial organism having a vector described herein comprising a nucleic acid described herein. Also provided anon-naturally occurring microbial organism having a nucleic acid described herein. In some embodiments, the nucleic acid is integrated into a chromosome of the organism. In some embodiments, the integration is site-specific. In an embodiment described herein, the nucleic acid is expressed. In some embodiments, provided herein is a non-naturally occurring microbial organism having a polypeptide described herein.

In some embodiments, the microbial organism is a species of bacteria, yeast or fungus. In some embodiments, the microbial organism is a species of bacteria, yeast or fungus. In some embodiments, the microbial organism is a species of yeast. In some embodiments, the microbial organism is a species of fungus.

In some embodiments, provided herein is a non-naturally occurring microbial organism that is a capable of producing more acetyl-phosphate, acetyl-CoA or a bioderived compound compared to a control microbial organism that does not having a recombinant nucleic acid that encodes an engineered phosphoketolase described herein. Such a microbial organism, in some embodiments, is capable of producing at least 10% more acetyl-phosphate, acetyl-CoA or a bioderived compound compared to the control microbial organism. In some embodiments, the microbial organism is capable of producing at least 20% more acetyl-phosphate, acetyl-CoA or a bioderived compound compared to the control microbial organism. In some embodiments, the microbial organism is capable of producing at least 30% more acetyl-phosphate, acetyl-CoA or a bioderived compound compared to the control microbial organism. In some embodiments, the microbial organism is capable of producing at least 40% more acetyl-phosphate, acetyl-CoA or a bioderived compound compared to the control microbial organism. In some embodiments, the microbial organism is capable of producing at least 50% more acetyl-phosphate, acetyl-CoA or a bioderived compound compared to the control microbial organism. In some embodiments, the microbial organism is capable of producing at least 60% more acetyl-phosphate, acetyl-CoA or a bioderived compound compared to the control microbial organism. In some embodiments, the microbial organism is capable of producing at least 70% more acetyl-phosphate, acetyl-CoA or a bioderived compound compared to the control microbial organism. In some embodiments, the microbial organism is capable of producing at least 80% more acetyl-phosphate, acetyl-CoA or a bioderived compound compared to the control microbial organism. In some embodiments, the microbial organism is capable of producing at least 90% more acetyl-phosphate, acetyl-CoA or a bioderived compound compared to the control microbial organism. In some embodiments, the microbial organism is capable of producing at least 1 fold more acetyl-phosphate, acetyl-CoA or a bioderived compound compared to the control microbial organism. In some embodiments, the microbial organism is capable of producing at least 1.1 fold more acetyl-phosphate, acetyl-CoA or a bioderived compound compared to the control microbial organism. In some embodiments, the microbial organism is capable of producing at least 1.2 fold more acetyl-phosphate, acetyl-CoA or a bioderived compound compared to the control microbial organism. In some embodiments, the microbial organism is capable of producing at least 1.3 fold more acetyl-phosphate, acetyl-CoA or a bioderived compound compared to the control microbial organism. In some embodiments, the microbial organism is capable of producing at least 1.4 fold more acetyl-phosphate, acetyl-CoA or a bioderived compound compared to the control microbial organism. In some embodiments, the microbial organism is capable of producing at least 1.5 fold more acetyl-phosphate, acetyl-CoA or a bioderived compound compared to the control microbial organism. In some embodiments, the microbial organism is capable of producing at least 1.6 fold more acetyl-phosphate, acetyl-CoA or a bioderived compound compared to the control microbial organism. In some embodiments, the microbial organism is capable of producing at least 1.7 fold more acetyl-phosphate, acetyl-CoA or a bioderived compound compared to the control microbial organism. In some embodiments, the microbial organism is capable of producing at least 1.8 fold more acetyl-phosphate, acetyl-CoA or a bioderived compound compared to the control microbial organism. In some embodiments, the microbial organism is capable of producing at least 1.9 fold more acetyl-phosphate, acetyl-CoA or a bioderived compound compared to the control microbial organism. In some embodiments, the microbial organism is capable of producing at least 2 fold more acetyl-phosphate, acetyl-CoA or a bioderived compound compared to the control microbial organism.

The subject matter described herein includes general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The non-naturally occurring microbial organisms described herein can be produced by introducing expressible nucleic acids encoding one or more of the enzymes or proteins participating in one or more acetyl-phosphate, acetyl-CoA or a bioderived compound biosynthetic pathways. Depending on the host microbial organism chosen for biosynthesis, nucleic acids for some or all of a particular acetyl-phosphate, acetyl-CoA or a bioderived compound biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve acetyl-phosphate, acetyl-CoA or a bioderived compound biosynthesis. Thus, a non-naturally occurring microbial organism described herein can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as acetyl-phosphate, acetyl-CoA or a bioderived compound.

Host microbial organisms can be selected from, and the non-naturally occurring microbial organisms generated in, for example, bacteria, yeast, fungus or any of a variety of other microorganisms applicable or suitable to fermentation processes. Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary bacterial methylotrophs include, for example, Bacillus, Methylobacterium, Methyloversatilis, Methylococcus, Methylocystis and Hyphomicrobium.

Similarly, exemplary species of yeast or fungi species include any species selected from the order Saccharomycetales, family Saccaromycetaceae, including the genera Saccharomyces, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. E. coli is a particularly useful host organism since it is a well characterized microbial organism suitable for genetic engineering. Other particularly useful host organisms include yeast such as Saccharomyces cerevisiae and yeasts or fungi selected from the genera Saccharomyces, Schizosaccharomyces, Schizochytrium, Rhodotorula, Thraustochytrium, Aspergillus, Kluyveromyces, Issatchenkia, Yarrowia, Candida, Pichia, Ogataea, Kuraishia, Hansenula and Komagataella. Useful host organisms include Saccharomyces cerevisiae, Schizosaccharomyces pombe, Hansenula polymorpha, Pichia methanolica, Candida boidinii, Kluyveromyces lactis, Kluyveromyces marxianus, Aspergillus terreus, Aspergillus niger, Pichia pastoris, Rhizopus arrhizus, Rhizopus oryzae, Yarrowia lipolytica, Issatchenkia orientalis and the like. It is understood that any suitable microbial host organism can be used to introduce metabolic and/or genetic modifications to produce a desired product.

Depending on the acetyl-phosphate, acetyl-CoA or bioderived compound biosynthetic pathway constituents of a selected host microbial organism, the non-naturally occurring microbial organisms described herein can include at least one exogenously expressed acetyl-phosphate, acetyl-CoA or a bioderived compound pathway-encoding nucleic acid and up to all encoding nucleic acids for one or more acetyl-phosphate, acetyl-CoA or a bioderived compound biosynthetic pathways. For example, acetyl-phosphate, acetyl-CoA or a bioderived compound biosynthesis can be established in a host deficient in a pathway enzyme or protein through exogenous expression of the corresponding encoding nucleic acid. In a host deficient in all enzymes or proteins of an acetyl-phosphate, acetyl-CoA or a bioderived compound pathway, exogenous expression of all enzyme or proteins in the pathway can be included, although it is understood that all enzymes or proteins of a pathway can be expressed even if the host contains at least one of the pathway enzymes or proteins.

Given the teachings and guidance provided herein, those skilled in the art will understand that the number of encoding nucleic acids to introduce in an expressible form will, at least, parallel the acetyl-phosphate, acetyl-CoA or bioderived compound pathway deficiencies of the selected host microbial organism. Therefore, a non-naturally occurring microbial organism described herein can have one, two, three, four, five, six, seven, eight, nine, ten, eleven, or twelve up to all nucleic acids encoding the enzymes or proteins constituting an acetyl-phosphate, acetyl-CoA or a bioderived compound biosynthetic pathway disclosed herein. In some embodiments, the non-naturally occurring microbial organisms also can include other genetic modifications that facilitate or optimize acetyl-phosphate, acetyl-CoA or a bioderived compound biosynthesis or that confer other useful functions onto the host microbial organism. One such other functionality can include, for example, augmentation of the synthesis of one or more of the acetyl-phosphate, acetyl-CoA or bioderived compound pathway precursors such as fructose-6-phosphate and xylulose-5-phosphate.

Generally, a host microbial organism is selected such that it produces the precursor of an acetyl-phosphate, acetyl-CoA or a bioderived compound pathway, either as a naturally produced molecule or as an engineered product that either provides de novo production of a desired precursor or increased production of a precursor naturally produced by the host microbial organism. For example, malonyl-CoA, acetoacetyl-CoA and pyruvate are produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of an acetyl-phosphate, acetyl-CoA or a bioderived compound pathway.

In some embodiments, a non-naturally occurring microbial organism described herein is generated from a host that contains the enzymatic capability to synthesize acetyl-phosphate, acetyl-CoA or a bioderived compound. In this specific embodiment it can be useful to increase the synthesis or accumulation of an acetyl-phosphate, acetyl-CoA or a bioderived compound pathway product to, for example, drive acetyl-phosphate, acetyl-CoA or a bioderived compound pathway reactions toward acetyl-phosphate, acetyl-CoA or a bioderived compound production. Increased synthesis or accumulation can be accomplished by, for example, expression (e.g., overexpression) of nucleic acids encoding an engineered phosphoketolase described herein and expression (e.g., overexpression) of an enzyme or enzymes and/or protein or proteins of the acetyl-phosphate, acetyl-CoA or bioderived compound pathway. Expression of the enzyme or enzymes and/or protein or proteins of the acetyl-phosphate, acetyl-CoA or bioderived compound pathway can occur, for example, through exogenous expression of the endogenous gene or genes, or through exogenous expression of the heterologous gene or genes. Therefore, naturally occurring organisms can be readily generated to be non-naturally occurring microbial organisms described herein, for example, producing acetyl-phosphate, acetyl-CoA or a bioderived compound, through overexpression of one, two, three, four, five, six, seven, eight, nine, ten, eleven, twelve, that is, up to all nucleic acids encoding acetyl-phosphate, acetyl-CoA or a bioderived compound biosynthetic pathway enzymes or proteins. In addition, a non-naturally occurring organism can be generated by mutagenesis of an endogenous gene that results in an increase in activity of an enzyme in the acetyl-phosphate, acetyl-CoA or bioderived compound biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. In some embodiments, the expression of an endogenous gene is manipulated, such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods described herein, any of the one or more recombinant and/or exogenous nucleic acids can be introduced into a microbial organism to produce a non-naturally occurring microbial organism described herein. The nucleic acids can be introduced so as to confer, for example, an acetyl-phosphate, acetyl-CoA or a bioderived compound biosynthetic pathway onto the microbial organism. Alternatively, encoding nucleic acids can be introduced to produce an intermediate microbial organism having the biosynthetic capability to catalyze some of the required reactions to confer acetyl-phosphate, acetyl-CoA or a bioderived compound biosynthetic capability. For example, a non-naturally occurring microbial organism having an acetyl-phosphate, acetyl-CoA or a bioderived compound biosynthetic pathway can comprise at least two exogenous nucleic acids encoding desired enzymes or proteins, such as the combination of an engineered phosphoketolase provided herein and a phosphotransacetylase, or alternatively an engineered phosphoketolase provided herein and a pyruvate formate lyase, or alternatively an engineered phosphoketolase provided herein and a pyruvate dehydrogenase, and the like. Thus, it is understood that any combination of two or more enzymes or proteins of a biosynthetic pathway can be included in a non-naturally occurring microbial organism described herein. Similarly, it is understood that any combination of three or more enzymes or proteins of a biosynthetic pathway can be included in a non-naturally occurring microbial organism described herein, for example, an engineered phosphoketolase provided herein, an acetate kinase and an acetyl-CoA transferase, synthetase, or ligase, and so forth, as desired, so long as the combination of enzymes and/or proteins of the desired biosynthetic pathway results in production of the corresponding desired product. Similarly, any combination of four, five, six, seven, eight, nine, ten, eleven, twelve or more enzymes or proteins of a biosynthetic pathway as disclosed herein can be included in a non-naturally occurring microbial organism described herein, as desired, so long as the combination of enzymes and/or proteins of the desired biosynthetic pathway results in production of the corresponding desired product.

In addition to the biosynthesis of acetyl-phosphate, acetyl-CoA or a bioderived compound as described herein, the non-naturally occurring microbial organisms and methods described herein also can be utilized in various combinations with each other and/or with other microbial organisms and methods well known in the art to achieve product biosynthesis by other routes. For example, one alternative to produce acetyl-phosphate, acetyl-CoA or a bioderived compound other than use of the acetyl-phosphate, acetyl-CoA or bioderived compound producers is through addition of another microbial organism capable of converting an acetyl-phosphate, acetyl-CoA or a bioderived compound pathway intermediate to acetyl-phosphate, acetyl-CoA or a bioderived compound. One such procedure includes, for example, the fermentation of a microbial organism that produces an acetyl-phosphate, acetyl-CoA or a bioderived compound pathway intermediate. The acetyl-CoA or the bioderived compound pathway intermediate can then be used as a substrate for a second microbial organism that converts the acetyl-phosphate, acetyl-CoA or bioderived compound pathway intermediate to acetyl-phosphate, acetyl-CoA or a bioderived compound. The acetyl-phosphate, acetyl-CoA or bioderived compound pathway intermediate can be added directly to another culture of the second organism or the original culture of the acetyl-phosphate, acetyl-CoA or bioderived compound pathway intermediate producers can be depleted of these microbial organisms by, for example, cell separation, and then subsequent addition of the second organism to the fermentation broth can be utilized to produce the final product without intermediate purification steps.

Given the teachings and guidance provided herein, those skilled in the art will understand that a wide variety of combinations and permutations exist for the non-naturally occurring microbial organisms and methods described herein together with other microbial organisms, with the co-culture of other non-naturally occurring microbial organisms having subpathways and with combinations of other chemical and/or biochemical procedures well known in the art to produce acetyl-phosphate, acetyl-CoA or a bioderived compound.

Similarly, it is understood by those skilled in the art that a host organism can be selected based on desired characteristics for introduction of one or more gene disruptions to increase synthesis or production of acetyl-phosphate, acetyl-CoA or a bioderived compound. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods described herein can be applied to any suitable host microorganism to identify the cognate metabolic alterations needed to construct an organism in a species of interest that will increase acetyl-phosphate, acetyl-CoA or a bioderived compound biosynthesis. In a particular embodiment, the increased production couples biosynthesis of acetyl-phosphate, acetyl-CoA or a bioderived compound to growth of the organism, and can obligatorily couple production of acetyl-phosphate, acetyl-CoA or a bioderived compound to growth of the organism if desired and as disclosed herein.

Sources of encoding nucleic acids for an acetyl-phosphate, acetyl-CoA or a bioderived compound pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Abies grandis, Acetobacter aceti, Acetobacter pasteurians, Achromobacter denitrificans, Acidaminococcus fermentans, Acinetobacter baumannii Naval-82, Acinetobacter baylyi, Acinetobacter calcoaceticus, Acinetobacter sp. ADPI, Acinetobacter sp. Strain M-1, Actinobacillus succinogenes, Actinobacillus succinogenes 130Z, Aeropyrum pernix, Agrobacterium tumefaciens, Alkaliphilus metalliredigenes QYF, Allochromatium vinosum DSM180, Aminomonas aminovorus, Amycolicicoccus subflavus DQS3-9A1, Anaerobiospirillum succiniciproducens, Anaerotruncus colihominis, Aquifex aeolicus VF5, Arabidopsis thaliana, Arabidopsis thaliana col, Archaeglubus fulgidus, Archaeoglobus fulgidus, Archaeoglobus fulgidus DSM 4304, Arthrobacter globiformis, Ascaris suum, Aspergillus fumigatus, Aspergillus nidulans, Aspergillus niger, Aspergillus niger CBS 513.88, Aspergillus terreus NIH2624, Atopobium parvulum DSM 20469, Azotobacter vinelandii DJ, Bacillus alcalophilus ATCC 27647, Bacillus azotoformans LMG 9581, Bacillus cereus, Bacillus cereus ATCC 14579, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus sphaericus, Bacillus subtilis, Bacteroides capillosus, Bifidobacterium animalis lactis, Bifidobacterium breve, Bifidobacterium dentium ATCC 27678, Bifidobacterium pseudolongum subsp. globosum, Bos taurus, Burkholderia cenocepacia, Burkholderia cepacia, Burkholderia multivorans, Burkholderia pyrrocinia, Burkholderia stabilis, Burkholderia thailandensis E264, Burkholderia xenovorans, butyrate-producing bacterium L2-50, Caenorhabditis elegans, Campylobacter curvus 525.92, Campylobacter jejuni, Candida albicans, Candida boidinii, Candida methylica, Candida parapsilosis, Candida tropicalis, Candida tropicalis MYA-3404, Carboxydothermus hydrogenoformans, Carboxydothermus hydrogenoformans Z-2901, Castellaniella defragrans, Caulobacter sp. AP07, Chlamydomonas reinhardtii, Chlorobium phaeobacteroides DSM266, Chlorobium limicola, Chlorobium tepidum, Chloroflexus aggregans DSM 9485, Chloroflexus aurantiacus, Chloroflexus aurantiacus J-10-fl, Citrobacter koseri ATCC BAA-895, Citrobacter youngae, Citrobacter youngae ATCC 29220, Clostridium acetobutylicum, Clostridium acetobutylicum ATCC 824, Clostridium acidurici, Clostridium aminobutyricum, Clostridium asparagiforme DSM15981, Clostridium beijerinckii, Clostridium beijerinckii NCIMB 8052, Clostridium beijerinckii NRRL B593, Clostridium beijerinckii, Clostridium bolteae ATCC BAA-613, Clostridium botulinum C str. Eklund, Clostridium carboxidivorans P7, Clostridium cellulolyticum H10, Clostridium cellulovorans 743B, Clostridium difficile, Clostridium difficile 630, Clostridium hiranonis DSM13275, Clostridium hylemonae DSM15053, Clostridium kluyveri, Clostridium kluyveri DSM 555, Clostridium ljungdahli, Clostridium ljungdahlii DSM, Clostridium ljungdahlii DSM13528, Clostridium methylpentosum DSM 5476, Clostridium novyi NT, Clostridium pasteurianum, Clostridium pasteurianum DSM525, Clostridium perfringens, Clostridium perfringens ATCC 13124, Clostridium perfringens str. 13, Clostridium phytofermentans ISDg, Clostridium propionicum, Clostridium saccharobutylicum, Clostridium saccharoperbutylacetonicum, Clostridium saccharoperbutylacetonicum N1-4, Clostridium tetani, Comamonas sp. CNB-1, Comamonas sp. CNB-1, Corynebacterium glutamicum, Corynebacterium glutamicum ATCC 13032, Corynebacterium glutamicum ATCC 14067, Corynebacterium glutamicum R, Corynebacterium sp., Corynebacterium sp. U-96, Corynebacterium variabile, Cryptosporidium parvum Iowa II, Cucumis sativus, Cupriavidus necator N-1, Cyanobium PCC7001, Deinococcus radiodurans RI, Desulfatibacillum alkenivorans AK-01, Desulfitobacterium hafniense, Desulfitobacterium metallireducens DSM15288, Desulfotomaculum reducens MI-1, Desulfovibrio africanus, Desulfovibrio africanus str. Walvis Bay, DesulfoVibrio desulfuricans G20, Desulfovibrio desulfuricans subsp. desulfuricans str. ATCC 27774, Desulfovibrio fructosovorans JJ, Desulfovibrio vulgaris str. Hildenborough, Dictyostelium discoideum AX4, Elizabethkingia meningoseptica, Enterococcus faecalis, Erythrobacter sp. NAP1, Escherichia coli C, Escherichia coli K12, Escherichia coli K-12 MG1655, Escherichia coli W, Eubacterium barkeri, Eubacterium hallii DSM 3353, Eubacterium rectale ATCC 33656, Euglena gracilis, Flavobacterium frigoris, Fusobacterium nucleatum, Fusobacterium nucleatum subsp. polymorphum ATCC 10953, Geobacillus sp. GHH01, Geobacillus sp. M10EXG, Geobacillus sp. Y4.1MC1, Geobacillus stearothermophilus, Geobacillus themodenitrificans NG80-2, Geobacillus thermoglucosidasius, Geobacter bemidjiensis Bem, Geobacter metallireducens GS-15, Geobacter sulfurreducens, Geobacter sulfurreducens PCA, Haemophilus influenza, Haemophilus influenzae, Haloarcula marismortui, Haloarcula marismortui ATCC 43049, Halobacterium salinarum, Hansenula polymorpha DL-1, Helicobacter pylori, Helicobacter pylori 26695, Heliobacter pylori, Homo sapiens, human gut metagenome, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Kluyveromyces lactis, Kluyveromyces lactis NRRL Y-1140, Lactobacillus acidophilus, Lactobacillus brevis ATCC 367, Lactobacillus paraplantarum, Lactococcus lactis, Leuconostoc mesenteroides, Lysinibacillus fusiformis, Lysinibacillus sphaericus, Malus×domestica, Mannheimia succiniciproducens, marine gamma proteobacterium HTCC2080, Marine metagenome JCVI_SCAF_1096627185304, Mesorhizobium loti MAFF303099, Metallosphaera sedula, Metarhizium acridum CQMa 102, Methanocaldococcus janaschii, Methanocaldococcus jannaschii, Methanosarcina acetivorans, Methanosarcina acetivorans C2A, Methanosarcina barkeri, Methanosarcina mazei, Methanosarcina mazei Tuc01, Methanosarcina thermophila, Methanothermobacter thermautotrophicus, Methylibium petroleiphilum PM1, Methylobacillus fagellatus, Methylobacillus fagellatus KT, Methylobacter marinus, Methylobacterium extorquens, Methylobacterium extorquens AM1, Methylococcus capsulatas, Methylomicrobium album BG8, Methylomonas aminofaciens, Methylovorus glucosetrophus SIP3-4, Methylovorus sp. MP688, Moorella thermoacetica, Mus musculus, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Mycoplasma pneumoniae M129, Natranaerobius thermophilus, Nectria haematococca mpVJ 77-13-4, Neurospora crassa, Nitrososphaera gargensis Ga9.2, Nocardia brasiliensis, Nocardia farcinica IFM 10152, Nocardia iowensis, Nocardia iowensis (sp. NRRL 5646), Nostoc sp. PCC 7120, Ogataea parapolymorpha DL-1 (Hansenula polymorpha DL-1), Organism, Oryctolagus cuniculus, Oxalobacter formigenes, Paenibacillus peoriae KCTC 3763, Paracoccus denitrificans, Pelobacter carbinolicus DSM2380, Pelotomaculum thermopropionicum, Penicillium chrysogenum, Perkinsus marinus ATCC 50983, Photobacterium profundum 3TCK, Picea abies, Pichia pastoris, Picrophilus torridus DSM9790, Pinus sabiniana, Plasmodium falciparum, Populus alba, Populus tremula×Populus alba, Porphyromonas gingivalis, Porphyromonas gingivalis W83, Propionibacterium acnes, Propionibacterium fredenreichii sp. shermanii, Pseudomonas aeruginosa, Pseudomonas aeruginosa PA01, Pseudomonas chlororaphis, Pseudomonas fluorescens, Pseudomonas knackmussii, Pseudomonas knackmussii (B13), Pseudomonas mendocina, Pseudomonas putida, Pseudomonas sp, Pseudomonas syringae pv. syringae B728a, Psychroflexus torquis ATCC 700755, Pueraria montana, Pyrobaculum aerophilum str. IM2, Pyrobaculum islandicum DSM 4184, Pyrococcus abyssi, Pyrococcus furiosus, Pyrococcus horikoshii OT3, Ralstonia eutropha, Ralstonia eutropha H16, Rattus norvegicus, Rhizobium leguminosarum, Rhodobacter capsulatus, Rhodobacter sphaeroides, Rhodobacter sphaeroides ATCC 17025, Rhodococcus opacus B4, Rhodococcus ruber, Rhodopseudomonas palustris, Rhodopseudomonas palustris CGA009, Rhodospirillum rubrum, Rhodospirillum rubrum ATCC 11170, Roseburia intestinalis L1-82, Roseburia inulinivorans, Roseburia sp. A2-183, Roseiflexus castenholzii, Rubrivivax gelatinosus, Ruminococcus obeum ATCC 29174, Saccharomyces cerevisiae, Saccharomyces cerevisiae s288c, Saccharomyces kluyveri, Saccharomyces serevisiae, Sachharomyces cerevisiae, Salmonella enteric, Salmonella enterica, Salmonella enterica subsp. Arizonae serovar, Salmonella enterica subsp. Enterica serovar Typhimurium str. LT2, Salmonella enterica Typhimurium, Salmonella typhimurium, Salmonella typhimurium LT2, Schizosaccharomyces pombe, Sebaldella termitidis ATCC 33386, Serratia proteamaculans, Shewanella oneidensis MR-1, Shigella flexneri, Sinorhizobium meliloti 1021, Solanum lycopersicum, Staphylococcus aureus, Stereum hirsutum FP-91666 SSI, Streptococcus mutans, Streptococcus pneumonia, Streptococcus pneumoniae, Streptococcus pyogenes ATCC 10782, Streptomyces anulatus, Streptomyces avermitilis, Streptomyces cinnamonensis, Streptomyces clavuligerus, Streptomyces coelicolor, Streptomyces coelicolor A3(2), Streptomyces griseus, Streptomyces griseus subsp. griseus NBRC 13350, Streptomyces sp CL190, Streptomyces sp. 2065, Streptomyces sp. ACT-1, Streptomyces sp. KO-3988, Sulfolobus acidocalarius, Sulfolobus acidocaldarius, Sulfolobus solfataricus, Sulfolobus solfataricus P-2, Sulfolobus sp. strain 7, Sulfolobus tokodaii, Sulfurimonas denitrificans, Sus scrofa, Synechococcus elongatus PCC 7942, Synechococcus sp. PCC 7002, Synechocystis str. PCC 6803, Syntrophobacter fumaroxidans, Thauera aromatica, Thermoanaerobacter brockii HTD4, Thermoanaerobacter sp. X514, Thermoanaerobacter tengcongensis MB4, Thermococcus kodakaraensis, Thermococcus litoralis, Thermoplasma acidophilum, Thermoproteus neutrophilus, Thermotoga maritima, Thermotoga maritime, Thermotoga maritime MSB8, Thermus thermophilus, Thiocapsa roseopersicina, Tolumonas auensis DSM9187, Treponema denticola, Trichomonas vaginalis G3, Triticum aestivum, Trypanosoma brucei, Tsukamurella paurometabola DSM 20162, Uncultured bacterium, uncultured organism, Vibrio cholera, Vibrio harveyi ATCC BAA-1116, Xanthobacter autotrophicus Py2, Yarrowia lipolytica, Yersinia frederiksenii, Yersinia intermedia, Yersinia intermedia ATCC 29909, Yersinia pestis, Zea mays, Zoogloea ramigera, Zymomonas mobilus, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite acetyl-phosphate, acetyl-CoA or a bioderived compound biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of acetyl-phosphate, acetyl-CoA or a bioderived compound described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative acetyl-phosphate, acetyl-CoA or a bioderived compound biosynthetic pathway exists in an unrelated species, acetyl-phosphate, acetyl-CoA or a bioderived compound biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods described herein can be applied to all microbial organisms using the cognate metabolic alterations to those exemplified herein to construct a microbial organism in a species of interest that will synthesize acetyl-phosphate, acetyl-CoA or a bioderived compound.

Methods for constructing and testing the expression levels of a non-naturally occurring acetyl-phosphate, acetyl-CoA or a bioderived compound-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered phosphoketolase as described herein and/or an exogenous nucleic acid encoding one or more enzymes or proteins involved in a pathway for production of acetyl-phosphate, acetyl-CoA or a bioderived compound as described herein can be introduced stably or transiently into a microbial organism using techniques well known in the art including, but not limited to, conjugation, electroporation, chemical transformation, transduction, transfection, and ultrasound transformation. For exogenous expression in E. coli or other prokaryotic cells, some nucleotide sequences in the genes or cDNAs of eukaryotic nucleic acids can encode targeting signals such as an N-terminal mitochondrial or other targeting signal, which can be removed before transformation into prokaryotic microbial organisms, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the microbial organisms. Thus, it is understood that appropriate modifications to a nucleotide sequence to remove or include a targeting sequence can be incorporated into a recombinant nucleic acid or an exogenous nucleic acid to impart desirable properties. Furthermore, genes can be subjected to codon optimization with techniques well known in the art to achieve optimized expression of the proteins.

An expression vector or vectors can be constructed to include a recombinant nucleic acid encoding an engineered phosphoketolase as described herein and/or an exogenous nucleic acid encoding one or more enzymes or proteins of an acetyl-phosphate, acetyl-CoA or a bioderived compound biosynthetic pathway as described herein operably linked to expression control sequences functional in the host organism. Expression vectors applicable for use in the microbial host organisms described herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more recombinant and/or exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of a recombinant or exogenous nucleic acid involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid or its corresponding gene product. It is understood by those skilled in the art that the recombinant and/or exogenous nucleic acid is expressed in a sufficient amount to produce the desired product, and it is further understood that expression levels can be optimized to obtain sufficient expression using methods well known in the art and as disclosed herein.

In some embodiments, provided herein is a method for producing a bioderived compound described herein. Such a method can comprise culturing the non-naturally occurring microbial organism as described herein under conditions and for a sufficient period of time to produce the bioderived compound. Thus, in some embodiments, provided herein is a method for producing a bioderived compound described herein comprising culturing a host cell described herein for a sufficient period of time to produce the bioderived compound. In another embodiment, method further includes separating the bioderived compound from other components in the culture. In this aspect, separating can include extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, depending on the bioderived compound, the method described herein may further include chemically converting a bioderived compound to the directed final compound. For example, in some embodiments, wherein the bioderived compound is butadiene, the method described herein can further include chemically dehydrating 1,3-butanediol, crotyl alcohol, or 3-buten-2-ol to produce the butadiene.

Suitable purification and/or assays to test for the production of acetyl-phosphate, acetyl-CoA or a bioderived compound can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the recombinant and/or exogenous nucleic acids can also be assayed using methods well known in the art.

The bioderived compound can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products described herein. For example, the bioderived compound producers can be cultured for the biosynthetic production of a bioderived compound disclosed herein. Accordingly, in some embodiments, provided herein is a culture medium having the bioderived compound or bioderived compound pathway intermediate described herein. In some aspects, the culture medium can also be separated from the non-naturally occurring microbial organisms described herein that produced the bioderived compound or bioderived compound pathway intermediate. Methods for separating a microbial organism from culture medium are well known in the art. Exemplary methods include filtration, flocculation, precipitation, centrifugation, sedimentation, and the like.

For the production of acetyl-phosphate, acetyl-CoA or a bioderived compound, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high acetyl-phosphate, acetyl-CoA or a bioderived compound yields.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium, can include, for example, any carbohydrate source which can supply a source of carbon to the non-naturally occurring microbial organism described herein. Such sources include, for example, sugars such as glucose, xylose, arabinose, galactose, mannose, fructose, sucrose and starch; or glycerol, alone as the sole source of carbon or in combination with other carbon sources described herein or known in the art. In one embodiment, the carbon source is a sugar. In one embodiment, the carbon source is a sugar-containing biomass. In some embodiments, the sugar is glucose. In one embodiment, the sugar is xylose. In another embodiment, the sugar is arabinose. In one embodiment, the sugar is galactose. In another embodiment, the sugar is fructose. In other embodiments, the sugar is sucrose. In one embodiment, the sugar is starch. In certain embodiments, the carbon source is glycerol. In some embodiments, the carbon source is crude glycerol. In one embodiment, the carbon source is crude glycerol without treatment. In other embodiments, the carbon source is glycerol and glucose. In another embodiment, the carbon source is methanol and glycerol. In one embodiment, the carbon source is carbon dioxide. In one embodiment, the carbon source is formate. In one embodiment, the carbon source is methane. In one embodiment, the carbon source is methanol. In certain embodiments, methanol is used alone as the sole source of carbon or in combination with other carbon sources described herein or known in the art. In a specific embodiment, the methanol is the only (sole) carbon source. In one embodiment, the carbon source is chemoelectro-generated carbon (see, e.g., Liao et al. (2012) Science 335:1596). In one embodiment, the chemoelectro-generated carbon is methanol. In one embodiment, the chemoelectro-generated carbon is formate. In one embodiment, the chemoelectro-generated carbon is formate and methanol. In one embodiment, the carbon source is a carbohydrate and methanol. In one embodiment, the carbon source is a sugar and methanol. In another embodiment, the carbon source is a sugar and glycerol. In other embodiments, the carbon source is a sugar and crude glycerol. In yet other embodiments, the carbon source is a sugar and crude glycerol without treatment. In one embodiment, the carbon source is a sugar-containing biomass and methanol. In another embodiment, the carbon source is a sugar-containing biomass and glycerol. In other embodiments, the carbon source is a sugar-containing biomass and crude glycerol. In yet other embodiments, the carbon source is a sugar-containing biomass and crude glycerol without treatment. In some embodiments, the carbon source is a sugar-containing biomass, methanol and a carbohydrate. Other sources of carbohydrate include, for example, renewable feedstocks and biomass. Exemplary types of biomasses that can be used as feedstocks in the methods provided herein include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms provided herein for the production of succinate and other pathway intermediates.

The non-naturally occurring microbial organisms described herein are constructed using methods well known in the art as exemplified herein to express a recombinant nucleic acid and/or one or more nucleic acids encoding an acetyl-phosphate, acetyl-CoA or a bioderived compound pathway enzyme or protein in sufficient amounts to produce acetyl-phosphate, acetyl-CoA or a bioderived compound. It is understood that the microbial organisms described herein are cultured under conditions sufficient to produce acetyl-phosphate, acetyl-CoA or a bioderived compound. Following the teachings and guidance provided herein, the non-naturally occurring microbial organisms described herein can achieve biosynthesis of acetyl-phosphate, acetyl-CoA or a bioderived compound compound resulting in intracellular concentrations between about 0.1-200 mM or more. Generally, the intracellular concentration of acetyl-phosphate, acetyl-CoA or a bioderived compound compound is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms described herein.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the acetyl-phosphate, acetyl-CoA or the bioderived compound producers can synthesize acetyl-phosphate, acetyl-CoA, or a bioderived compound at intracellular concentrations of 5-10 mM or more as well as all other concentrations exemplified herein. It is understood that, even though the above description refers to intracellular concentrations, acetyl-phosphate, acetyl-CoA or a bioderived compound producing microbial organisms can produce acetyl-phosphate, acetyl-CoA or a bioderived compound intracellularly and/or secrete the product into the culture medium.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

The culture conditions can include, for example, liquid culture procedures as well as fermentation and other large scale culture procedures. As described herein, particularly useful yields of the biosynthetic products described herein can be obtained under anaerobic or substantially anaerobic culture conditions.

As described herein, one exemplary growth condition for achieving biosynthesis of acetyl-phosphate, acetyl-CoA or a bioderived compound includes anaerobic culture or fermentation conditions. In certain embodiments, the non-naturally occurring microbial organisms described herein can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of acetyl-phosphate, acetyl-CoA or a bioderived compound. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of acetyl-phosphate, acetyl-CoA or a bioderived compound. Generally, and as with non-continuous culture procedures, the continuous and/or near-continuous production of acetyl-phosphate, acetyl-CoA or a bioderived compound will include culturing a non-naturally occurring acetyl-phosphate, acetyl-CoA or a bioderived compound producing organism described herein in sufficient nutrients and medium to sustain and/or nearly sustain growth in an exponential phase. Continuous culture under such conditions can include, for example, growth or culturing for 1 day, 2, 3, 4, 5, 6 or 7 days or more. Additionally, continuous culture can include longer time periods of 1 week, 2, 3, 4 or 5 or more weeks and up to several months. Alternatively, organisms described herein can be cultured for hours, if suitable for a particular application. It is to be understood that the continuous and/or near-continuous culture conditions also can include all time intervals in between these exemplary periods. It is further understood that the time of culturing the microbial organism described herein is for a sufficient period of time to produce a sufficient amount of product for a desired purpose.

Fermentation procedures are well known in the art. Briefly, fermentation for the biosynthetic production of acetyl-phosphate, acetyl-CoA or a bioderived compound can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the acetyl-phosphate, acetyl-CoA or bioderived compound producers described herein for continuous production of substantial quantities of acetyl-phosphate, acetyl-CoA or a bioderived compound, the acetyl-phosphate, acetyl-CoA or bioderived compound producers also can be, for example, simultaneously subjected to chemical synthesis and/or enzymatic procedures to convert the product to other compounds or the product can be separated from the fermentation culture and sequentially subjected to chemical and/or enzymatic conversion to convert the product to other compounds, if desired.

In some embodiments, the carbon feedstock and other cellular uptake sources such as phosphate, ammonia, sulfate, chloride and other halogens can be chosen to alter the isotopic distribution of the atoms present in acetyl-phosphate, acetyl-CoA or a bioderived compound or any acetyl-phosphate, acetyl-CoA or a bioderived compound pathway intermediate. The various carbon feedstock and other uptake sources enumerated above will be referred to herein, collectively, as “uptake sources.” Uptake sources can provide isotopic enrichment for any atom present in the acetyl-phosphate, acetyl-CoA, bioderived compound or pathway intermediate, or for side products generated in reactions diverging away from an acetyl-phosphate, acetyl-CoA or a bioderived compound pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

Accordingly, in some embodiments, provided herein is an acetyl-phosphate, acetyl-CoA or a bioderived compound or an acetyl-phosphate, acetyl-CoA or a bioderived compound pathway intermediate that has a carbon-12, carbon-13, and carbon-14 ratio that reflects an atmospheric carbon, also referred to as environmental carbon, uptake source. For example, in some aspects the acetyl-phosphate, acetyl-CoA or bioderived compound or an acetyl-phosphate, acetyl-CoA or a bioderived compound pathway intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, provided herein is an acetyl-phosphate, acetyl-CoA or a bioderived compound or an acetyl-phosphate, acetyl-CoA or a bioderived compound pathway intermediate that has a carbon-12, carbon-13, and carbon-14 ratio that reflects petroleum-based carbon uptake source. In this aspect, the acetyl-phosphate, acetyl-CoA or bioderived compound or an acetyl-phosphate, acetyl-CoA or a bioderived compound pathway intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, provided herein is an acetyl-phosphate, acetyl-CoA or a bioderived compound or an acetyl-phosphate, acetyl-CoA or a bioderived compound pathway intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present disclosure relates to the biologically produced acetyl-phosphate, acetyl-CoA, a bioderived compound or pathway intermediate as disclosed herein, and to the products derived therefrom, wherein the acetyl-phosphate, acetyl-CoA or bioderived compound or an acetyl-phosphate, acetyl-CoA or a bioderived compound pathway intermediate has a carbon-12, carbon-13, and carbon-14 isotope ratio of about the same value as the CO₂ that occurs in the environment. For example, in some aspects provided herein is a bioderived acetyl-phosphate, acetyl-CoA or a bioderived compound or a bioderived acetyl-phosphate, acetyl-CoA or a bioderived compound intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from a bioderived compound or a bioderived compound pathway intermediate as disclosed herein, wherein the bioderived product is chemically modified to generate a final product. Methods of chemically modifying a bioderived product of a bioderived compound, or an intermediate thereof, to generate a desired product are well known to those skilled in the art, as described herein. The disclosure further provides biobased products having a carbon-12 versus carbon-13 versus carbon-14 isotope ratio of about the same value as the CO₂ that occurs in the environment, wherein the biobased products are generated directly from or in combination with bioderived compound or a bioderived compound pathway intermediate as disclosed herein.

The disclosure further provides a composition comprising bioderived compound described herein and a compound other than the bioderived compound. The compound other than the bioderived product can be a cellular portion, for example, a trace amount of a cellular portion of, or can be fermentation broth or culture medium, or a purified or partially purified fraction thereof produced in the presence of, a non-naturally occurring microbial organism described herein. The composition can comprise, for example, a reduced level of a byproduct when produced by an organism having reduced byproduct formation, as disclosed herein. The composition can comprise, for example, bioderived compound, or a cell lysate or culture supernatant of a microbial organism described herein.

In certain embodiments, provided herein is a composition comprising a bioderived compound provided herein produced by culturing a non-naturally occurring microbial organism described herein. In some embodiments, the composition further comprises a compound other than said bioderived compound. In certain embodiments, the compound other than said bioderived compound is a trace amount of a cellular portion of a non-naturally occurring microbial organism described herein.

### SEQUENCES

The sequences in the following Table illustrate amino acid sequences and nucleotide sequences that can be used to generate the compositions and perform the methods described herein. As needed, an RNA sequence can be readily deduced from the DNA sequence.

| SEQ | | |
|---|---|---|
| ID | | |
| NO: | Description | Amino Acid or Nucleotide Sequence |
| 1. | fructose-6- | MMTSPVIGTPWKKLNAPVSEEAIEGVDKYWRAANYLSIG |
| | phosphate | QIYLRSNPLMKEPFTREDVKHRLVGHWGTTPGLNFLIGH |
| | phosphoketolase | INRLIADHQQNTVIIMGPGHGGPAGTAQSYLDGTYTEYF |
| | from | PNITKDEAGLQKFFRQFSYPGGIPSHYAPETPGSIHEGG |
| | Bifidobacterium | ELGYALSHAYGAVMNNPSLFVPAIVGDGEAETGPLATGW |
| | adolescentis | QSNKLINPRTDGIVLPILHLNGYKIANPTILSRISDEEL |
| | | HEFFHGMGYEPYEFVAGEDNEDHLSIHRRFAELFETVED |
| | | EICDIKAAAQTDDMTRPFYPMIIFRTPKGWTCPKFIDGK |
| | | KTEGSWRSHQVPLASARDTEAHFEVLKNWLESYKPEELF |
| | | DENGAVKPEVTAFMPTGELRIGENPNANGGRIREELKLP |
| | | KLEDYEVKEVAEYGHGWGQLEATRRLGVYTRDIIKNNPD |
| | | SFRIFGPDETASNRLQAAYDVINKQWDAGYLSAQVDEHM |
| | | AVTGQVTEQLSEHQMEGFLEGYLLTGRHGIWSSYESFVH |
| | | VIDSMLNQHAKWLEATVREIPWRKPISSMNLLVSSHVWR |
| | | QDHNGFSHQDPGVTSVLLNKCENNDHVIGIYFPVDSNML |
| | | LAVAEKCYKSTNKINAIIAGKQPAATWLTLDEARAELEK |
| | | GAAEWKWASNVKSNDEAQIVLAATGDVPTQEIMAAADKL |
| | | DAMGIKFKVVNVVDLVKLQSAKENNEALSDEEFAELFTE |
| | | DKPVLFAYHSYARDVRGLIYDRPNHDNENVHGYEEQGST |
| | | TTPYDMVRVNNIDRYELQAEALRMIDADKYADKINELEA |
| | | FRQEAFQFAVDNGYDHPDYTDWVYSGVNTNKQGAISATA |
| | | ATAGDNE |
| 2. | xylulose-5- | MAKTLGTPWQKLGHEVPASELEGVDLYWRASNYLSVGQI |
| | phosphate/ | YLRSNPLMRADFVDDKTGEARDEGRPDVKHRLVGHWGTT |
| | fructose-6- | PGINFLFGHVNRLIADHNQNAIFLMGPGHGGPAGTAQSL |
| | phosphate | LDGTYREIRPDITNDEAGLQKFFRQFSYPGGIPSHFAPE |
| | phosphoketolase | TPGSIHEGGELGYTLSHAYGAVMDNPSLLAVAVVGDGES |
| | from Collinsella | ETGPLATSWQSNKLVNPATDGIVLPILHLNGYKIANPTI |
| | aerofaciens | LARVSDEELTKFFEGMGYKPHFFIAGEDDESHASIHERE |
| | | AALFEQVEDEICDIKATAQAQAAAGETVVRPAYPMIVER |
| | | TPKGWTCPKQIDGKKTEDSWRAHQVPLASAKDTHEHERV |
| | | LREWLRSYKPEELFTPEGQVRPEVTAFMPTGELRIGANP |
| | | NANGGKVRRELELPDIHAHEIPVAEKGHGWGSTEAARVE |
| | | GEYTADVLAKNMDDFRIFGPDETASNRLQAAYRVTKKOW |
| | | DAGFYEDEANDELLAGSGKVVEQLSEHQCEGFLEAYVLT |
| | | GRSGVWSSYESFVHVVDSMVNQHCKWLEATKREIPWRAP |
| | | ISGLNILLSSHVWRQDHNGFSHQDPGFIDLLLNKANDTH |
| | | IVNAYYPADANMALAVAERVYQSTDCVNAIFCGKOPAPT |
| | | FQTVDEAKAELAEGVANWEWASTADSLGEADVVVATCGD |
| | | VPTLEALAATDMLRELGIKVWFVNVVDLLKIQNVCENDQ |
| | | AISDERWAELFGCGEKPVLFAFHAYAGTIRRLIWNRPGH |
| | | DAFRVHGYEEKGSTTTPFDMLRLNNMDRWALAADVLRMV |
| | | DAVKFAEQIDEWEAFRTEAFEFACDEGEDHPAFTDWVWP |
| | | DAAAATAADGALSATQLTAGDNE |
| 3. | fructose-6- | atgatgacgagcccagttattgggaccccttggaaaaag |
| | phosphate | ttaaacgccccggtaagtgaggaagctatagaaggagtg |
| | phosphoketolase | gataaatactggagggcggcaaattatttgtcgatcggc |
| | from | caaatttatctccgatctaacccgcttatgaaagagccc |
| | Bifidobacterium | tttacacgtgaagacgtcaaacatcgcctggtgggtcac |
| | adolescentis | tggggcactaccccgggtctgaatttcctgatcggccat |
| | | attaatcggctaatcgcggatcaccagcagaacacggtt |
| | | attatcatggggccgggtcatggcgggccagcaggtacc |
| | | gcccaatcatacctggatggaacgtataccgaatacttt |
| | | cccaatataactaaagacgaagcgggcttgcagaagttc |
| | | tttcgccagttcagctatccgggtggcattccgtcccac |
| | | tatgcccctgaaactccaggatctatccatgagggcggt |
| | | gaactgggatacgcattgtcccacgcttatggcgcggta |
| | | atgaacaacccaagcctgtttgtgcctgcgattgtcggt |
| | | gatggtgaagccgaaaccggcccgctcgctacaggctgg |
| | | caaagtaacaaactgattaatccgcgtaccgatggtatc |
| | | gttctcccgattctgcatttaaacggctataaaatcgcg |
| | | aacccaacgattttatcgcgcatctcagacgaggagctg |
| | | catgagttttttcatgggatgggctacgaaccctatgaa |
| | | tttgtggctgggttcgataatgaggatcacttaagtata |
| | | caccgtcgtttcgcagaacttttcgaaaccgtgtttgac |
| | | gagatttgcgacattaaagccgccgcgcagactgacgat |
| | | atgacgcgcccgttttatccgatgatcatcttccgaacc |
| | | ccgaagggctggacatgtccgaaatttatcgatggtaaa |
| | | aagactgagggttcttggcgctcgcaccaagttcctctg |
| | | gcaagcgcccgtgataccgaagcgcatttcgaagtcctg |
| | | aaaaattggcttgaaagctacaaacctgaggaattgttt |
| | | gacgaaaatggcgcagtcaaacctgaagtgacggcattc |
| | | atgccaactggcgagttacgaattggtgaaaacccgaat |
| | | gccaatgggggccgcattagggaagagctaaaattaccg |
| | | aagctggaagattacgaggtaaaggaagtcgcggaatat |
| | | ggacatggttgggggcagctggaggcgacccgtcgcctg |
| | | ggagtgtacacacgtgatatcattaaaaacaacccagac |
| | | agctttcgcatctttggcccagatgaaaccgcttcaaac |
| | | cgtttacaggccgcatatgatgttaccaacaaacaatgg |
| | | gacgctggctatttgagtgctcaggtggatgaacacatg |
| | | gcggtgacgggtcaggtgaccgaacagctgagtgaacat |
| | | caaatggaaggctttcttgagggttatctgttgaccggt |
| | | cggcacggtatctggtcctcgtatgagtctttcgtacac |
| | | gtcattgatagcatgctcaatcagcatgccaagtggctg |
| | | gaagctactgttcgggaaatcccgtggcgcaaaccgatt |
| | | tcatctatgaacctgctggttagtagccatgtttggagg |
| | | caggaccataatggattctcgcaccaagatcccggtgta |
| | | acgtccgtacttctgaacaaatgctttaacaatgaccat |
| | | gtaataggaatttacttccccgtggatagcaacatgctc |
| | | ttggcggttgcggaaaaatgttacaaatccactaacaaa |
| | | atcaatgctatcatcgcaggaaagcagccagccgcgaca |
| | | tggctgacgttagatgaagctagagcggaactggagaaa |
| | | ggcgcagcagaatggaaatgggcctccaatgttaagtct |
| | | aacgatgaagctcagattgtgcttgcggccaccggtgac |
| | | gtcccgacccaagaaattatggccgcagccgataagtta |
| | | gacgccatgggcatcaaatttaaagtcgtaaatgtggtt |
| | | gatctggtgaaactccaatctgcgaaggaaaataacgag |
| | | gctttgtcagacgaagagtttgcggaactgttcaccgaa |
| | | gataaaccggtcctgttcgcctaccattcgtacgcgcgc |
| | | gatgttcgtgggctgatttatgatcgtccaaaccatgac |
| | | aattttaatgtccacgggtatgaagaacagggtagtacg |
| | | acaacgccgtacgatatggtgcgggtaaataatatagat |
| | | cgttatgagctgcaagcagaggcgctacgcatgatcgac |
| | | gcggataaatacgccgacaaaattaatgaattagaagct |
| | | tttcgccaggaagctttccagtttgcagtcgataacggc |
| | | tatgatcatccagactacactgactgggtgtatagcggg |
| | | gtgaatactaacaaacagggtgcgattagtgcaaccgcc |
| | | gccaccgccggcgacaatgaa |
| 4. | xylulose-5- | atggcaaaaaccttagggactccgtggcagaagttgggt |
| | phosphate/ | cacgaagtacccgcgagcgagcttgaaggcgtcgatctg |
| | fructose-6- | tactggcgtgcctccaactatctgtcagttggacaaata |
| | phosphate | tatctgaggtctaatccactgatgcgcgctgactttgtg |
| | phosphoketolase | gatgataaaacaggcgaagcccgcgacttcggtcgtccg |
| | from Collinsella | gatgtgaaacatcggctcgtcggccattggggtacgacc |
| | aerofaciens | cctggcatcaactttctgttcgggcacgtgaatcgattg |
| | | attgcggaccataaccagaacgcaatctttctaatggga |
| | | ccgggccacggtggcccagcgggtacggcacagtcgctc |
| | | ctggacgggacctaccgcgagattagaccggatattact |
| | | aatgatgaagccggccttcaaaaattctttcgtcagttt |
| | | agctatccgggtggtatcccgagtcatttcgctccagag |
| | | acacctggatcgattcatgaagggggagaactaggctac |
| | | accttaagccacgcgtatggcgcggttatggacaatccg |
| | | tctttgctggcggtcgccgtagtgggtgatggtgagagt |
| | | gaaacgggacctcttgcgacctcctggcagagcaacaaa |
| | | ctggttaatccggccacggatggcattgtgctgcccatc |
| | | cttcatctgaacggctacaagattgctaacccgaccatc |
| | | ctcgcccgtgtgtccgatgaagaactgactaaatttttc |
| | | gagggcatgggttataaaccccatttctttattgcaggg |
| | | ttcgatgacgaatcacacgcttcgatccacgagcgcttt |
| | | gcggcgctattcgaacaagtctttgacgagatttgcgat |
| | | ataaaggccacggcacaggcccaagcggcggcaggggaa |
| | | acagtagtacgccctgcatatccgatgattgtttttcgt |
| | | acaccaaaaggctggacctgtccgaaacagatcgacggg |
| | | aaaaaaaccgaagattcttggcgcgcacaccaggttccg |
| | | ctggcttccgccaaagatactcatgagcatttccgcgtg |
| | | ctgcgtgaatggttacggagctacaaacccgaagaattg |
| | | tttacccccgagggtcaagtgcgtccggaggtcaccgct |
| | | ttcatgcctacgggtgaactgcgtataggtgccaatccg |
| | | aacgcaaatggcggcaaagtgcgccgagaattagaactc |
| | | ccggatatacacgcgcatgaaatcccagttgcggaaaag |
| | | ggccatgggtggggctcgaccgaggctgctcgggtgttt |
| | | ggagaatataccgctgatgtactggccaagaacatggac |
| | | gattttcgcatttttggtccagatgaaacagccagtaac |
| | | cgactgcaagcggcttatagggtcacgaaaaaacagtgg |
| | | gatgcgggtttctacgaagatgaggcaaatgacgagttg |
| | | ttagccgggagcgggaaggtggtggagcagttatcagaa |
| | | catcaatgcgaaggctttttagaagcatacgtcctgacg |
| | | ggtcgcagtggtgtttggagcagttacgaaagcttcgtc |
| | | cacgtcgtggactccatggttaaccagcactgtaagtgg |
| | | ctggaagcgaccaaacgagaaattccgtggagagcccct |
| | | atcagcggcttgaatattcttctgtcgagtcacgtctgg |
| | | cgccaggaccataacggattttcgcaccaagatccaggc |
| | | tttatcgatcttttattaaataaggcaaatgatacccat |
| | | atcgtaaacgcctattacccggcggacgcgaacatggct |
| | | ctggccgtggcggagcgtgtctatcagtctacggactgc |
| | | gttaatgcgattttttgcggaaaacagcccgcaccgact |
| | | ttccaaacggtggatgaagccaaagccgaactggccgag |
| | | ggcgtagcgaactgggaatgggcatcaactgcggactca |
| | | ctgggggaagcagacgtcgtggttgcgacctgtggggat |
| | | gtacctaccctggaagccctcgcggctactgacatgcta |
| | | cgtgaactgggcatcaaagtctggttcgtaaatgttgtt |
| | | gacctgttgaaaatccagaatgtctgtgaaaatgatcaa |
| | | gcgatttcagatgagcggtgggccgagctgtttgggtgc |
| | | ggcgaaaaaccggtgcttttcgcgtttcatgcgtatgcg |
| | | ggtacaattcgtcggctgatctggaaccgtccgggacat |
| | | gatgcgtttagagtacacggttatgaggaaaaaggttct |
| | | accacaaccccgtttgatatgctacgcttgaacaacatg |
| | | gatcgctgggccctcgcagccgatgtgctgcgcatggtt |
| | | gatgctgttaaattcgcggagcaaattgacgaatgggaa |
| | | gcattcagaacggaagcattcgaatttgcctgcgatgaa |
| | | ggctttgatcacccggccttcactgattgggtgtggccc |
| | | gacgccgccgcggcaaccgctgccgacggtgcgctgagc |
| | | gcgacacagctcacggctggcgataatgaa |

It is understood that modifications which do not substantially affect the activity of the various embodiments of this disclosure are also provided within the definition described herein provided herein. Accordingly, the following examples are intended to illustrate but not limit the present disclosure.

### Example 1

This example describes generation of engineered phosphoketolases with desirable properties, including increased activity in: catalyzing the conversion of fructose-6-phosphate to acetyl-phosphate; catalyzing the conversion of xylulose-5-phosphate to acetyl-phosphate; or catalyzing the conversion of fructose-6-phosphate to acetyl-phosphate and xylulose-5-phosphate to acetyl-phosphate.

Mutagenesis techniques were used to generate engineered phosphoketolases based on one of two template phosphoketolases (the fructose-6-phosphate phosphoketolase from Bifidobacterium adolescentis (strain ATCC 15703 DSM 20083 NCTC 11814 E194a) (“H2”; UniProtKB Accession No. A1A185_BIFAA; SEQ ID NOs: 1 and 3) orthe xylulose-5-phosphate/fructose-6-phosphate phosphoketolase from Collinsella aerofaciens (“D3”; UniProt KB Accession No. A0A174LGR4_9ACTN; SEQ ID NOs: 2 and 4)). Engineered phosphoketolase were generated using rational protein engineering. Structural homology models were constructed for H2 and D3. The substrate docking with Xu5P and F6P were also performed. In total, 2000 variants were designed. There are 1031 constructs designed based on H2, and 969 based on D3. 1999 constructs were received from DNA synthesis. The variants were then tested for XPK and/or FPK activity in in vitro primary and secondary screens as described below.

An in vitro primary screen was used to assess the FPK activity of the library variants. The library variants were split into two batches and subjected to screening using the D-erythrose-4-phosphate dehydrogenase (E4PDH)-based colorimetric assay for FPK activity described below. In total, 259 unique variants were selected with the standardized rate cutoff value of 0.6 for further confirmation in a secondary screening. The rate cutoff value was determined by a ratio of the average normalized enzyme rate:average normalized positive control rate. The normalized average reaction rate unit is mAbs/min/OD600. Thus, in order to identify the rate, the normalized reaction rate for the positive control (normalized by culture OD) and then the average normalized rate for positive control were determined, and the normalized rate for each enzyme candidate (normalized by culture OD) and the average normalized rate with replicates were determined.

An in vitro primary screen was also used to assess the XPK activity of library variants. The library variants were split into two batches and subjected to screening using the α-glycerophosphate-3-phosphate dehydrogenase (α-GDH) based colorimetric assay for XPK activity described below. In total, 414 unique variants were selected with the standardized rate cutoff value of 1.1 for further confirmation in a secondary screening.

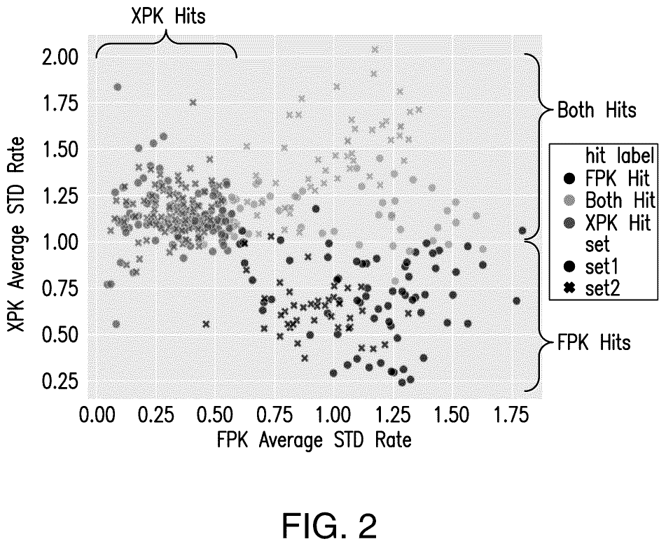

Further data analysis on hits from the primary screening for both FPK and XPK activities showed that the variants can be grouped into three main buckets as illustrated in FIG. 2. These three buckets were variants with: 1) good XPK activity, but poor in FPK (left side, denoted with “XPK hits”), 2) good FPK activity, but poor in XPK (lower right quadrant, denoted with “FPK hits”); or 3) both good XPK and FPK activities (upper right quadrant, denoted with “Both hits”).

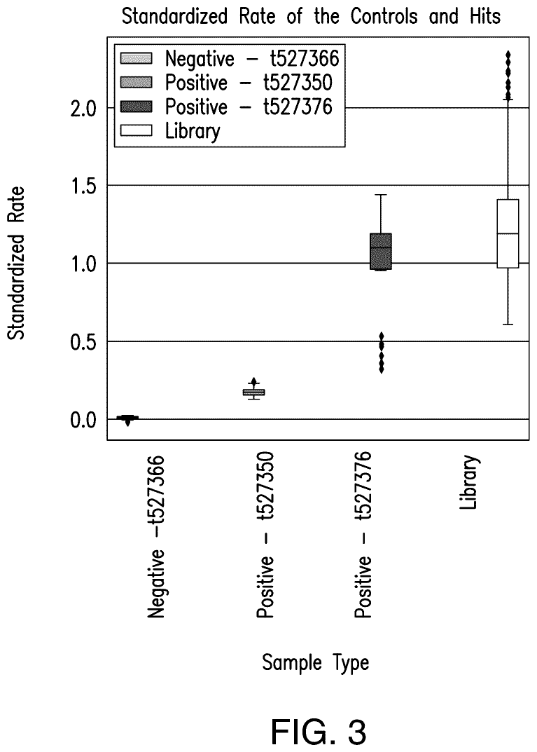

Following the primary screen, 259 unique variants from the primary screening for FPK activity were subjected to secondary screening for hit confirmation. In the secondary screening, the FPK assay was altered by lowering F6P concentration from 5 mM to 2 mM for challenging the Kₘ of the hit enzymes. The library performance was illustrated in FIG. 3.

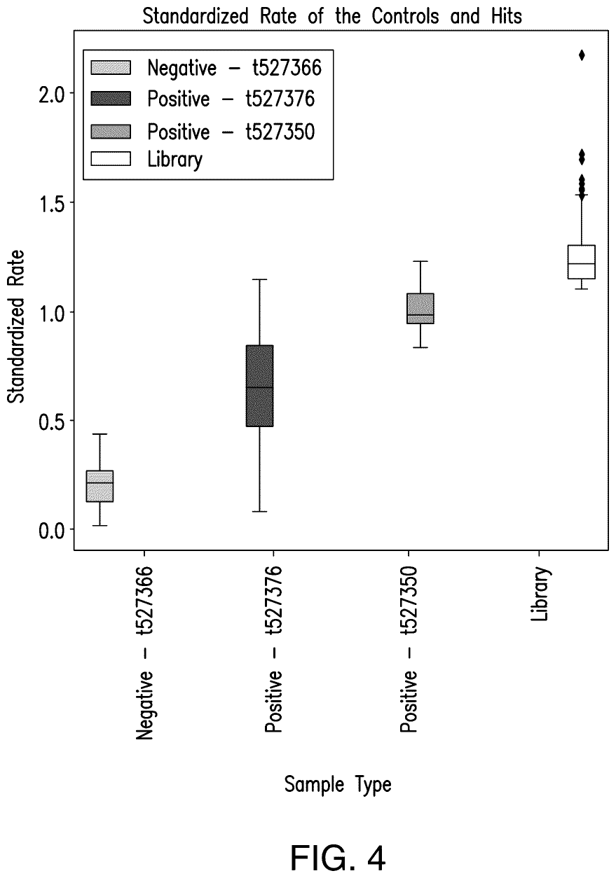

414 unique variants from the primary screening for XPK activity were also subjected to secondary screening for hit confirmation. In the secondary screening, XPK assay was run in the same condition as in primary screening. The library performance is illustrated in FIG. 4.

It was found that alterations around the active site are more permissible for XPK activity as compared to FPK activity. It was also found that alterations in the active site tended to reduce FPK activity.

The results of these primary and secondary screens, including identifying the activity of select variants, are showing Table 1 and Table 2.

TABLE 1
Exemplary Engineered Phosphoketolases Using H2 (SEQ ID NO: 1)
as the Template with Corresponding Catalytic FPK or XPK Activity
and Positional Relationship of Alternation to Active Site

| Variant | | | | | | Active |
|---|---|---|---|---|---|---|
| # | Residue | Position | Alteration | PK Assay | Activity | Site |
| 24 | M | 49 | L | FPK | + | |
| 9 | H | 60 | P | FPK | ++ | |
| 40 | V | 63 | L | FPK | + | Yes |
| 82 | V | 63 | L | XPK | ++ | Yes |
| 64 | G | 64 | A | XPK | ++ | Yes |
| 66 | G | 64 | S | XPK | ++ | Yes |
| 89 | H | 65 | Y | XPK | ++ | Yes |
| 10 | G | 77 | A | FPK | ++ | |
| 5 | T | 104 | L | FPK | ++ | |
| 33 | T | 104 | V | FPK | + | |
| 30 | Y | 136 | F | FPK | + | |
| 112 | Y | 136 | F | XPK | + | |
| 74 | G | 156 | A | XPK | ++ | Yes |
| 63 | L | 158 | I | XPK | ++ | Yes |
| 71 | L | 158 | V | XPK | ++ | Yes |
| 101 | L | 158 | M | XPK | ++ | Yes |
| 124 | L | 158 | A | XPK | + | Yes |
| 21 | S | 174 | D | FPK | + | |
| 6 | L | 200 | F | FPK | ++ | |
| 13 | I | 208 | A | FPK | ++ | |
| 60 | K | 219 | C | XPK | ++ | Yes |
| 65 | K | 219 | N | XPK | ++ | Yes |
| 76 | K | 219 | D | XPK | ++ | Yes |
| 87 | K | 219 | S | XPK | ++ | Yes |
| 90 | K | 219 | M | XPK | ++ | Yes |
| 98 | K | 219 | E | XPK | ++ | Yes |
| 72 | I | 220 | M | XPK | ++ | Yes |
| 19 | A | 221 | Q | FPK | + | Yes |
| 32 | A | 221 | C | FPK | + | Yes |
| 42 | A | 221 | T | FPK | + | Yes |
| 43 | A | 221 | V | XPK | ++ | Yes |
| 46 | A | 221 | R | XPK | ++ | Yes |
| 47 | A | 221 | N | XPK | ++ | Yes |
| 49 | A | 221 | M | XPK | ++ | Yes |
| 58 | A | 221 | H | XPK | ++ | Yes |
| 75 | A | 221 | E | XPK | ++ | Yes |
| 80 | A | 221 | G | XPK | ++ | Yes |
| 99 | A | 221 | C | XPK | ++ | Yes |
| 102 | A | 221 | T | XPK | ++ | Yes |
| 18 | L | 267 | A | FPK | ++ | |
| 116 | L | 267 | A | XPK | + | |
| 41 | C | 276 | R | FPK | + | |
| 67 | C | 276 | K | XPK | ++ | |
| 97 | C | 276 | R | XPK | ++ | |
| 4 | C | 305 | G | FPK | ++ | |
| 119 | C | 305 | G | XPK | + | |
| 115 | F | 335 | L | XPK | + | |
| 1 | F | 364 | L | FPK | ++ | |
| 121 | F | 364 | L | XPK | + | |
| 22 | M | 365 | A | FPK | + | |
| 28 | I | 372 | M | FPK | + | |
| 104 | I | 372 | M | XPK | ++ | |
| 27 | N | 377 | H | FPK | + | |
| 45 | W | 407 | R | XPK | ++ | |
| 31 | T | 420 | L | FPK | + | |
| 114 | T | 420 | L | XPK | + | |
| 50 | P | 436 | A | XPK | ++ | Yes |
| 51 | P | 436 | V | XPK | ++ | Yes |
| 77 | P | 436 | S | XPK | ++ | Yes |
| 79 | P | 436 | C | XPK | ++ | Yes |
| 93 | P | 436 | E | XPK | ++ | Yes |
| 84 | E | 438 | D | XPK | ++ | Yes |
| 88 | E | 438 | T | XPK | ++ | Yes |
| 54 | A | 440 | I | XPK | ++ | Yes |
| 55 | A | 440 | K | XPK | ++ | Yes |
| 20 | N | 442 | Q | FPK | + | Yes |
| 53 | N | 442 | A | XPK | ++ | Yes |
| 57 | N | 442 | D | XPK | ++ | Yes |
| 69 | N | 442 | T | XPK | ++ | Yes |
| 73 | N | 442 | H | XPK | ++ | Yes |
| 78 | N | 442 | M | XPK | ++ | Yes |
| 83 | N | 442 | V | XPK | ++ | Yes |
| 85 | N | 442 | C | XPK | ++ | Yes |
| 94 | N | 442 | S | XPK | ++ | Yes |
| 100 | N | 442 | I | XPK | ++ | Yes |
| 11 | A | 447 | V | FPK | ++ | |
| 48 | L | 478 | I | XPK | ++ | Yes |
| 62 | L | 478 | M | XPK | ++ | Yes |
| 17 | W | 499 | F | FPK | ++ | Yes |
| 16 | S | 504 | A | FPK | ++ | Yes |
| 81 | S | 504 | A | XPK | ++ | Yes |
| 86 | F | 505 | M | XPK | ++ | Yes |
| 91 | S | 542 | A | XPK | ++ | Yes |
| 92 | S | 542 | G | XPK | ++ | Yes |
| 123 | S | 542 | W | XPK | + | Yes |
| 125 | S | 542 | I | XPK | + | Yes |
| 52 | Q | 547 | E | XPK | ++ | Yes |
| 56 | Q | 547 | A | XPK | ++ | Yes |
| 44 | D | 548 | T | XPK | ++ | Yes |
| 61 | D | 548 | I | XPK | ++ | Yes |
| 68 | D | 548 | E | XPK | ++ | Yes |
| 70 | D | 548 | V | XPK | ++ | Yes |
| 95 | D | 548 | M | XPK | ++ | Yes |
| 96 | D | 548 | N | XPK | ++ | Yes |
| 105 | D | 548 | P | XPK | ++ | Yes |
| 120 | D | 548 | G | XPK | + | Yes |
| 118 | H | 549 | Y | XPK | + | Yes |
| 7 | H | 572 | D | FPK | ++ | |
| 12 | H | 572 | E | FPK | ++ | |
| 113 | H | 572 | E | XPK | + | |
| 15 | M | 584 | T | FPK | ++ | |
| 23 | M | 584 | S | FPK | + | |
| 25 | M | 584 | C | FPK | + | |
| 106 | M | 584 | S | XPK | ++ | |
| 122 | M | 584 | T | XPK | + | |
| 26 | Y | 593 | L | FPK | + | |
| 117 | Y | 593 | L | XPK | + | |
| 29 | T | 611 | Q | FPK | + | |
| 38 | E | 621 | H | FPK | + | |
| 35 | A | 627 | G | FPK | + | |
| 110 | A | 627 | G | XPK | ++ | |
| 39 | A | 641 | P | FPK | + | |
| 111 | A | 641 | P | XPK | ++ | |
| 2 | N | 687 | H | FPK | ++ | |
| 109 | N | 687 | H | XPK | ++ | |
| 8 | M | 765 | R | FPK | ++ | |
| 59 | D | 767 | P | XPK | ++ | |
| 14 | F | 781 | Q | FPK | ++ | |
| 36 | F | 781 | K | FPK | + | |
| 103 | F | 781 | R | XPK | ++ | |
| 107 | F | 781 | K | XPK | ++ | |
| 108 | F | 781 | Q | XPK | ++ | |
| 3 | F | 786 | K | FPK | ++ | |
| 34 | F | 786 | R | FPK | + | |
| 37 | F | 788 | Y | FPK | + | |

“+” = greater than 0.5 to 1 fold increase in activity relative to control
“++” = greater than 1 fold increase in activity relative to control
“Yes” = position is located within 12 angstroms from a docked fructose 6-phosphate substrate

TABLE 2
Exemplary Engineered Phosphoketolases Using D3 (SEQ ID NO: 2)
as the Template with Corresponding Catalytic FPK or XPK Activity
and Positional Relationship of Alternation to Active Site

| Variant | | | | | | Active |
|---|---|---|---|---|---|---|
| # | Residue | Position | Alteration | PK Assay | Activity | Site |
| 126 | S | 43 | D | FPK | ++ | |
| 138 | S | 43 | D | XPK | ++ | |
| 177 | H | 69 | P | XPK | + | |
| 131 | G | 73 | A | XPK | ++ | Yes |
| 154 | T | 113 | V | XPK | ++ | |
| 167 | T | 113 | L | XPK | + | |
| 149 | Y | 145 | F | XPK | ++ | |
| 153 | L | 167 | I | XPK | ++ | Yes |
| 134 | S | 183 | D | XPK | ++ | |
| 169 | A | 230 | Q | XPK | + | Yes |
| 170 | A | 230 | H | XPK | + | Yes |
| 155 | L | 276 | A | XPK | ++ | |
| 148 | Y | 306 | W | XPK | ++ | |
| 139 | Q | 336 | S | XPK | ++ | Yes |
| 129 | F | 349 | L | FPK | ++ | |
| 157 | F | 349 | L | XPK | ++ | |
| 135 | M | 379 | A | XPK | ++ | |
| 127 | W | 420 | R | FPK | ++ | |
| 160 | W | 420 | R | XPK | ++ | |
| 140 | P | 449 | A | XPK | ++ | Yes |
| 156 | E | 451 | I | XPK | ++ | Yes |
| 174 | A | 460 | V | XPK | + | |
| 143 | W | 513 | F | XPK | ++ | Yes |
| 132 | R | 560 | K | XPK | ++ | Yes |
| 146 | R | 560 | L | XPK | ++ | Yes |
| 162 | R | 560 | T | XPK | ++ | Yes |
| 165 | R | 560 | M | XPK | ++ | Yes |
| 172 | R | 560 | Y | XPK | + | Yes |
| 175 | R | 560 | Q | XPK | + | Yes |
| 176 | R | 560 | G | XPK | + | Yes |
| 178 | R | 560 | E | XPK | + | Yes |
| 130 | D | 562 | H | XPK | ++ | Yes |
| 144 | D | 562 | G | XPK | ++ | Yes |
| 145 | D | 562 | F | XPK | ++ | Yes |
| 152 | D | 562 | W | XPK | ++ | Yes |
| 161 | D | 562 | C | XPK | ++ | Yes |
| 163 | D | 562 | N | XPK | ++ | Yes |
| 168 | D | 562 | T | XPK | + | Yes |
| 171 | D | 562 | I | XPK | + | Yes |
| 166 | H | 585 | D | XPK | ++ | |
| 137 | Y | 606 | L | XPK | ++ | |
| 159 | A | 614 | V | XPK | ++ | |
| 150 | Q | 620 | K | XPK | ++ | Yes |
| 141 | E | 634 | H | XPK | ++ | |
| 164 | N | 700 | H | XPK | ++ | |
| 142 | M | 779 | R | XPK | ++ | |
| 133 | D | 781 | P | XPK | ++ | |
| 128 | F | 795 | R | FPK | ++ | |
| 136 | F | 795 | R | XPK | ++ | |
| 151 | F | 795 | Q | XPK | ++ | |
| 158 | F | 800 | K | XPK | ++ | |
| 173 | F | 800 | R | XPK | + | |
| 147 | F | 802 | Y | XPK | ++ | |

“+” = greater than 0.5 to 1 fold increase in activity relative to control
“++” = greater than 1 fold increase in activity relative to control
“Yes” = position is located within 12 angstroms from a docked fructose 6-phosphate substrate

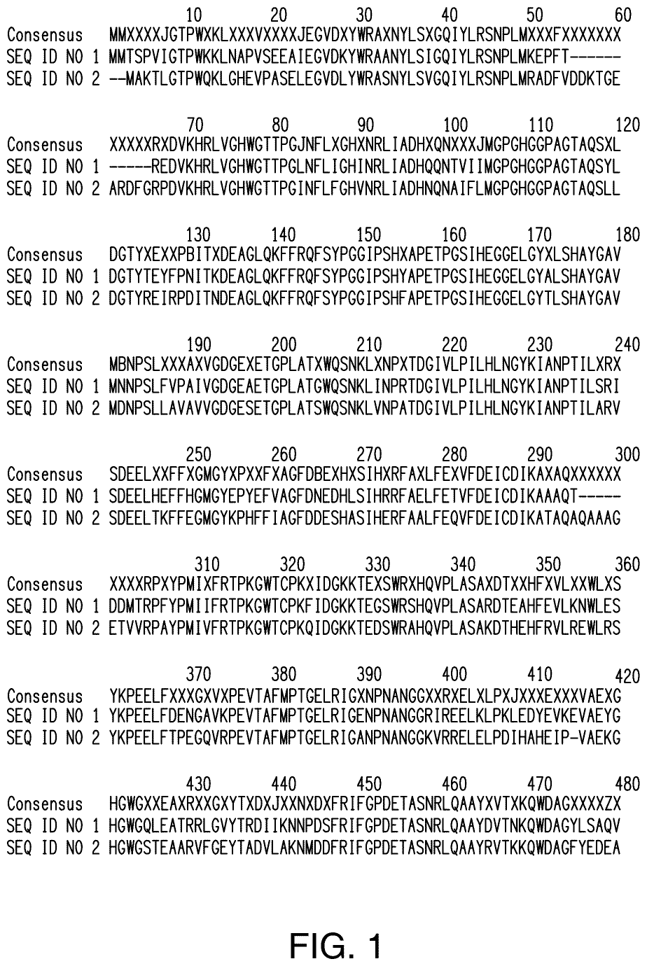

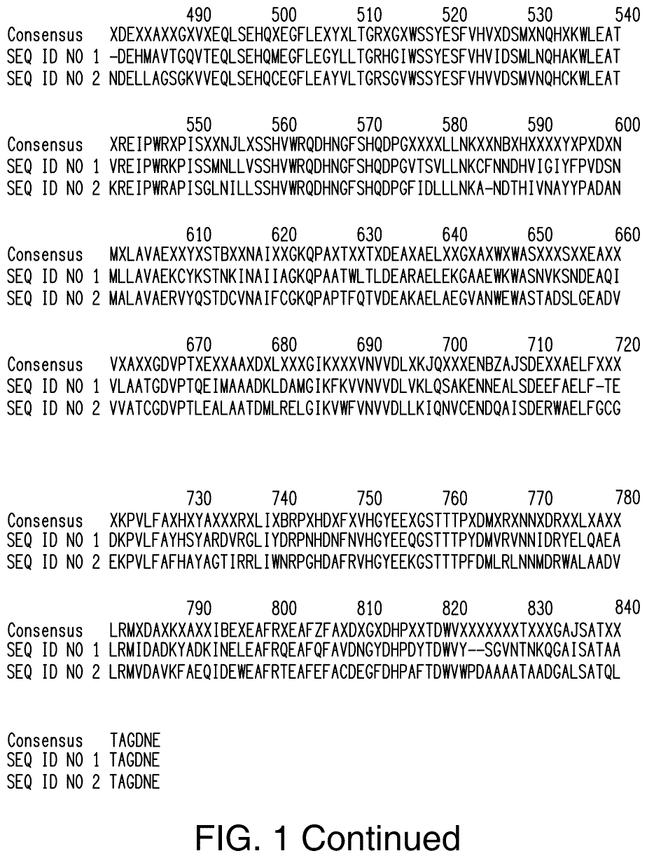

An amino acid alignment was performed using Geneious prime, a sequence viewer that permits amino acid viewing of multiple sequences. FIG. 1 provides an exemplary alignment between H2 and D3. Based on this and/or similar alignments, additional exemplary alternations were identified. Table 3 provides additional exemplary alternations for D3 using H2 as the template, and Table 4 provides addition exemplary alternations for H2 using D3 as the template.

TABLE 3
Residue Engineering Starting from H2 (SEQ ID NO: 1).

| H2 Template | | | D3 Alteration | | |
|---|---|---|---|---|---|
| Variant | | | Corresponding | Corresponding | |
| # | Position | Residue | Position | Residue | Alteration |
| 24 | 49 | M | 47 | M | L |
| 9 | 60 | H | 69 | H | P |
| 40 | 63 | V | 72 | V | L |
| 82 | 63 | V | 72 | V | L |
| 64 | 64 | G | 73 | G | A |
| 66 | 64 | G | 73 | G | S |
| 89 | 65 | H | 74 | H | Y |
| 10 | 77 | G | 86 | G | A |
| 5 | 104 | T | 113 | T | L |
| 33 | 104 | T | 113 | T | V |
| 30 | 136 | Y | 145 | Y | F |
| 112 | 136 | Y | 145 | Y | F |
| 74 | 156 | G | 165 | G | A |
| 63 | 158 | L | 167 | L | I |
| 71 | 158 | L | 167 | L | V |
| 101 | 158 | L | 167 | L | M |
| 124 | 158 | L | 167 | L | A |
| 21 | 174 | S | 183 | S | D |
| 6 | 200 | L | 209 | L | F |
| 13 | 208 | I | 217 | I | A |
| 60 | 219 | K | 228 | K | C |
| 65 | 219 | K | 228 | K | N |
| 76 | 219 | K | 228 | K | D |
| 87 | 219 | K | 228 | K | S |
| 90 | 219 | K | 228 | K | M |
| 98 | 219 | K | 228 | K | E |
| 72 | 220 | I | 229 | I | M |
| 19 | 221 | A | 230 | A | Q |
| 32 | 221 | A | 230 | A | C |
| 42 | 221 | A | 230 | A | T |
| 43 | 221 | A | 230 | A | V |
| 46 | 221 | A | 230 | A | R |
| 47 | 221 | A | 230 | A | N |
| 49 | 221 | A | 230 | A | M |
| 58 | 221 | A | 230 | A | H |
| 75 | 221 | A | 230 | A | E |
| 80 | 221 | A | 230 | A | G |
| 99 | 221 | A | 230 | A | C |
| 102 | 221 | A | 230 | A | T |
| 18 | 267 | L | 276 | L | A |
| 116 | 267 | L | 276 | L | A |
| 41 | 276 | C | 285 | C | R |
| 67 | 276 | C | 285 | C | K |
| 97 | 276 | C | 285 | C | R |
| 4 | 305 | C | 319 | C | G |
| 119 | 305 | C | 319 | C | G |
| 115 | 335 | F | 349 | F | L |
| 1 | 364 | F | 378 | F | L |
| 121 | 364 | F | 378 | F | L |
| 22 | 365 | M | 379 | M | A |
| 28 | 372 | I | 386 | I | M |
| 104 | 372 | I | 386 | I | M |
| 27 | 377 | N | 391 | N | H |
| 45 | 407 | W | 420 | W | R |
| 31 | 420 | T | 433 | T | L |
| 114 | 420 | T | 433 | T | L |
| 50 | 436 | P | 449 | P | A |
| 51 | 436 | P | 449 | P | V |
| 77 | 436 | P | 449 | P | S |
| 79 | 436 | P | 449 | P | C |
| 93 | 436 | P | 449 | P | E |
| 84 | 438 | E | 451 | E | D |
| 88 | 438 | E | 451 | E | T |
| 54 | 440 | A | 453 | A | I |
| 55 | 440 | A | 453 | A | K |
| 20 | 442 | N | 455 | N | Q |
| 53 | 442 | N | 455 | N | A |
| 57 | 442 | N | 455 | N | D |
| 69 | 442 | N | 455 | N | T |
| 73 | 442 | N | 455 | N | H |
| 78 | 442 | N | 455 | N | M |
| 83 | 442 | N | 455 | N | V |
| 85 | 442 | N | 455 | N | C |
| 94 | 442 | N | 455 | N | S |
| 100 | 442 | N | 455 | N | I |
| 11 | 447 | A | 460 | A | V |
| 48 | 478 | L | 492 | L | I |
| 62 | 478 | L | 492 | L | M |
| 17 | 499 | W | 513 | W | F |
| 16 | 504 | S | 518 | S | A |
| 81 | 504 | S | 518 | S | A |
| 86 | 505 | F | 519 | F | M |
| 91 | 542 | S | 556 | S | A |
| 92 | 542 | S | 556 | S | G |
| 123 | 542 | S | 556 | S | W |
| 125 | 542 | S | 556 | S | I |
| 52 | 547 | Q | 561 | Q | E |
| 56 | 547 | Q | 561 | Q | A |
| 44 | 548 | D | 562 | D | T |
| 61 | 548 | D | 562 | D | I |
| 68 | 548 | D | 562 | D | E |
| 70 | 548 | D | 562 | D | V |
| 95 | 548 | D | 562 | D | M |
| 96 | 548 | D | 562 | D | N |
| 105 | 548 | D | 562 | D | P |
| 120 | 548 | D | 562 | D | G |
| 118 | 549 | H | 563 | H | Y |
| 7 | 572 | H | 585 | H | D |
| 12 | 572 | H | 585 | H | E |
| 113 | 572 | H | 585 | H | E |
| 15 | 584 | M | 597 | M | T |
| 23 | 584 | M | 597 | M | S |
| 25 | 584 | M | 597 | M | C |
| 106 | 584 | M | 597 | M | S |
| 122 | 584 | M | 597 | M | T |
| 26 | 593 | Y | 606 | Y | L |
| 117 | 593 | Y | 606 | Y | L |
| 29 | 611 | T | 624 | T | Q |
| 38 | 621 | E | 634 | E | H |
| 35 | 627 | A | 640 | A | G |
| 110 | 627 | A | 640 | A | G |
| 39 | 641 | A | 654 | A | P |
| 111 | 641 | A | 654 | A | P |
| 2 | 687 | N | 700 | N | H |
| 109 | 687 | N | 700 | N | H |
| 8 | 765 | M | 779 | M | R |
| 59 | 767 | D | 781 | D | P |
| 14 | 781 | F | 795 | F | Q |
| 36 | 781 | F | 795 | F | K |
| 103 | 781 | F | 795 | F | R |
| 107 | 781 | F | 795 | F | K |
| 108 | 781 | F | 795 | F | Q |
| 3 | 786 | F | 800 | F | K |
| 34 | 786 | F | 800 | F | R |
| 37 | 788 | F | 802 | F | Y |

TABLE 4
Residue Engineering Starting from D3 (SEQ ID NO: 2).

| D3 Template | | | H2 Alteration | | |
|---|---|---|---|---|---|
| Variant | | | Corresponding | Corresponding | |
| # | Position | Residue | Position | Residue | Alteration |
| 126 | 43 | S | 45 | S | D |
| 138 | 43 | S | 45 | S | D |
| 177 | 69 | H | 60 | H | P |
| 131 | 73 | G | 64 | G | A |
| 154 | 113 | T | 104 | T | V |
| 167 | 113 | T | 104 | T | L |
| 149 | 145 | Y | 136 | Y | F |
| 153 | 167 | L | 158 | L | I |
| 134 | 183 | S | 174 | S | D |
| 169 | 230 | A | 221 | A | Q |
| 170 | 230 | A | 221 | A | H |
| 155 | 276 | L | 267 | L | A |
| 148 | 306 | Y | 292 | Y | W |
| 139 | 336 | Q | 322 | Q | S |
| 129 | 349 | F | 335 | F | L |
| 157 | 349 | F | 335 | F | L |
| 135 | 379 | M | 365 | M | A |
| 127 | 420 | W | 407 | W | R |
| 160 | 420 | W | 407 | W | R |
| 140 | 449 | P | 436 | P | A |
| 156 | 451 | E | 438 | E | I |
| 174 | 460 | A | 447 | A | V |
| 143 | 513 | W | 499 | W | F |
| 132 | 560 | R | 546 | R | K |
| 146 | 560 | R | 546 | R | L |
| 162 | 560 | R | 546 | R | T |
| 165 | 560 | R | 546 | R | M |
| 172 | 560 | R | 546 | R | Y |
| 175 | 560 | R | 546 | R | Q |
| 176 | 560 | R | 546 | R | G |
| 178 | 560 | R | 546 | R | E |
| 130 | 562 | D | 548 | D | H |
| 144 | 562 | D | 548 | D | G |
| 145 | 562 | D | 548 | D | F |
| 152 | 562 | D | 548 | D | W |
| 161 | 562 | D | 548 | D | C |
| 163 | 562 | D | 548 | D | N |
| 168 | 562 | D | 548 | D | T |
| 171 | 562 | D | 548 | D | I |
| 166 | 585 | H | 572 | H | D |
| 137 | 606 | Y | 593 | Y | L |
| 159 | 614 | A | 601 | A | V |
| 150 | 620 | Q | 607 | Q | K |
| 141 | 634 | E | 621 | E | H |
| 164 | 700 | N | 687 | N | H |
| 142 | 779 | M | 765 | M | R |
| 133 | 781 | D | 767 | D | P |
| 128 | 795 | F | 781 | F | R |
| 136 | 795 | F | 781 | F | R |
| 151 | 795 | F | 781 | F | Q |
| 158 | 800 | F | 786 | F | K |
| 173 | 800 | F | 786 | F | R |
| 147 | 802 | F | 788 | F | Y |

Further biochemical characterization of selected variants was conducted and the FPK activity of these variants were assessed. 12 hits were selected from the secondary screening results of the phosphoketolase library screen for in vitro biochemical characterization. All 12 of the hits selected are variants based on SEQ ID NO: 1. The kinetics parameters measured during biochemical characterization are summarized below in Table 5. The FPK kinetics assay was based on the E4PDH based Colorimetric Assay described herein for the 12 hits and the two controls (SEQ ID NO: 1 and SEQ ID NO: 2). Results with the Hydroxymate based Colorimetric Assay described herein are shown in Table 5 for comparison.

TABLE 5
Kinetics parameters from biochemical characterization studies

| SEQ ID NO or | | | | | |
|---|---|---|---|---|---|
| Alteration in | kcat, | Km, | kcat/ | | Active |
| SEQ ID NO: 1 | s\hat{ }(⁻¹) | mM | Km | Assay | Site? |
| SEQ ID NO: 2 | 19.57 | 9.312 | 2.101589 | Hydroxymate | |
| | | | | assay | |
| SEQ ID NO: 2 | 7.824 | 27.91 | 0.28033 | E4PDH assay | |
| SEQ ID NO: 1 | 5.933 | 1.041 | 5.699328 | Hydroxymate | |
| | | | | assay | |
| SEQ ID NO: 1 | 30.07 | 4.892 | 6.14677 | E4PDH assay | |
| H239R | 57.94 | 2.432 | 23.82401 | E4PDH assay | |
| M483C | 34.01 | 2.299 | 14.79339 | E4PDH assay | |
| Y804W | 30.76 | 2.86 | 10.75524 | E4PDH assay | |
| H257P | 25.01 | 2.422 | 10.32618 | E4PDH assay | |
| Q759L | 35.57 | 3.596 | 9.891546 | E4PDH assay | |
| C567S | 26.37 | 3.046 | 8.657255 | E4PDH assay | |
| S504A | 26.27 | 3.347 | 7.84882 | E4PDH assay | Yes |
| Y799I | 33.64 | 4.53 | 7.426049 | E4PDH assay | |
| H60P | 36.72 | 6.685 | 5.492895 | E4PDH assay | |
| D548T | 3.94 | 0.7512 | 5.244941 | E4PDH assay | Yes |
| A221N | 5.764 | 1.791 | 3.218314 | E4PDH assay | Yes |
| V323I | 24.64 | 9.262 | 2.660333 | E4PDH assay | Yes |

“Yes” = alteration position in SEQ ID NO: 1 is located within 12 angstroms from a docked fructose 6-phosphate substrate

### Materials and Methods

### α-GDH Based Colorimetric Assay for XPK Activity

5 μL/well of thawed glycerol stocks of PK transformants was stamped into 500 μL/well of LB media with 50 mg/L Kanamycin in half height deepwell plates and sealed with AeraSeals. Samples were incubated at 37° C. and shaken at 1000 revolutions per minute (RPM) in 80% humidity for overnight. 50 μL/well of the resulting cultures was stamped into 450 μL/well of autoinduction medium (ZYM-5052) in half-height deepwell plates and sealed with AeraSeals. Cultures were incubated at 37° C. and shaken at 1000 RPM in 80% humidity for 4 hours. Then, the shaker temperature was decreased to 28° C. for overnight culturing. 10 μL/well of the resulting production cultures was stamped into 190 μL/well Phosphate Buffered Saline (PBS) in 96-well flat bottom plates. Optical measurements were taken on a plate reader, with absorbance measured at 600 nm. 125 μL/well cultures were retrieved and spinned down. The cell pellets were stored at −80° C. freezer until further use.

For screening, the frozen samples were fully thawed at room temperature for about one hour. The cells were lysed in a 125 μL/well lysis buffer. The resultant lysate was used for in vitro assays. The product of XPK is coupled to NADH consumption via a two-step enzymatic reaction of its product with triosephosphate isomerase and glycerophosphate dehydrogenase. NADH consumption over time was measured by absorbance at 340 nm. The reactions are listed below.

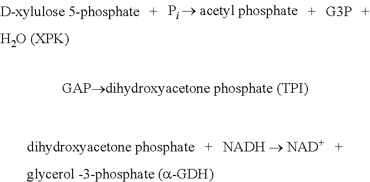

5 μL of cell lysate was added into the 45 μL of the reaction buffer in a well of a 96 clear bottom, half area black plate. The reaction buffer contained appropriate amounts of TPI and α-GDH, and 0.2 mM Xu5P substrate. For each reaction, a 10-min kinetics reading at 340 nm was performed.

### Hydroxymate Based Colorimetric Assay for FPK Activity

The product of FPK is coupled to ferric-hydroxamate complex production via hydroxamate reactions of its product with hydroxylamine and ferric chloride. Ferric-hydroxamate complex production was measured by absorbance at 520 nM. This is an end-point assay. The reactions are listed below.

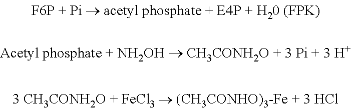

Cell lysate preparation was the same described as above. One typical reaction contained 7.5 uL lysate and 67.5 μL reaction buffer. Reaction buffer contained 1 mM of NADH and 5 mM of F6P substrate for primary screening. 2 mM of F6P was used in secondary screening in reaction buffer 1. The reaction in a plate was incubated at room temperature for 30 minutes. After the 30-min incubation, 25 μL of 2 M hydroxylamine in Tris-HCl solution was added into one reaction to initiate the hydroxamate reaction. The reaction was incubated at room temperature for 10 minutes and then sent to the plate reader for absorption at 520 nm.

### E4PDH Based Colorimetric Assay for FPK Activity

The product of FPK is coupled to NADH production via a one-step enzymatic reaction of its product with E4PDH. NADH production overtime was measured by absorbance at 340 nm. The reactions are listed below.

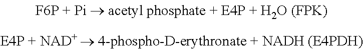

5 μL of half diluted cell lysate was added into 45 μL of reaction buffer. The reaction buffer contained an approximate amount of E4PDH enzyme, 1 mM NADH and 5 mM F6P as substrate for primary screening. 2 mM F6P was used for secondary screening. The reaction was recorded for a 10-min kinetics reading at 340 nm at room temperature.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit described herein.

What is claimed is:

1. An engineered phosphoketolase comprising a variant of amino acid sequence SEQ ID NO: 1 or 2 or a functional fragment thereof, wherein the engineered phosphoketolase comprises one or more alterations at a position described in Tables 1, 2, 3, and/or 4.

2. The engineered phosphoketolase of claim 1, wherein the engineered phosphoketolase is capable of:
(a) catalyzing the conversion of fructose-6-phosphate to acetyl-phosphate;
(b) catalyzing the conversion of xylulose-5-phosphate to acetyl-phosphate; or
(c) catalyzing the conversion of fructose-6-phosphate to acetyl-phosphate and xylulose-5-phosphate to acetyl-phosphate.

3. The engineered phosphoketolase of claim 2, wherein the engineered phosphoketolase is capable of forming erythrose-4-phosphate and/or glyceraldehyde-3-phosphate.

4. The engineered phosphoketolase of any one of claims 1 to 3, wherein the engineered phosphoketolase comprises an activity that is at least 0.5, at least 1.0, at least 1.5, or at least 2.0 fold higher than the activity of a phosphoketolase consisting of the amino acid sequence of SEQ ID NO: 1 or 2.

5. The engineered phosphoketolase of any one of claims 1 to 4, wherein the engineered phosphoketolase comprises one or more amino acid substitutions at a residue corresponding to position 49, 60, 63, 64, 65, 77, 104, 136, 156, 158, 174, 200, 208, 219, 220, 221, 239, 257, 267, 276, 305, 323, 335, 364, 365, 372, 377, 407, 420, 436, 438, 440, 442, 447, 478, 483, 499, 504, 505, 542, 547, 548, 549, 567, 572, 584, 593, 611, 621, 627, 641, 687, 759, 765, 767, 781, 786, 788, 799, and/or 804 in SEQ ID NO: 1.

6. The engineered phosphoketolase of any one of claims 1 to 4, wherein the engineered phosphoketolase comprises one or more amino acid substitutions at a residue corresponding to position 49, 60, 63, 77, 104, 136, 174, 200, 208, 221, 239, 257, 267, 276, 305, 323, 364, 365, 372, 377, 420, 442, 447, 483, 499, 504, 548, 567, 572, 584, 593, 611, 621, 627, 641, 687, 759, 765, 781, 786, 788, 799, and/or 804 in SEQ ID NO: 1.

7. The engineered phosphoketolase of any one of claims 1 to 4, wherein the engineered phosphoketolase comprises one or more amino acid substitutions at a residue corresponding to position 63, 64, 65, 136, 156, 158, 219, 220, 221, 267, 276, 305, 335, 364, 372, 407, 420, 436, 438, 440, 442, 478, 504, 505, 542, 547, 548, 549, 572, 584, 593, 627, 641, 687, 767, and/or 781 in SEQ ID NO: 1.

8. The engineered phosphoketolase of any one of claims 1 to 4, wherein the engineered phosphoketolase comprises one or more amino acid substitutions at a residue corresponding to position 63, 136, 221, 267, 276, 305, 364, 372, 420, 504, 572, 584, 593, 627, 641, 687, and/or 781 in SEQ ID NO: 1.

9. The engineered phosphoketolase of any one of claims 1 to 4, wherein the engineered phosphoketolase comprises one or more amino acid substitutions at a residue corresponding to position 60, 64, 104, 136, 158, 174, 221, 267, 335, 365, 407, 436, 447, 499, 548, 572, 593, 621, 687, 765, 767, 781, 786, and/or 788 in SEQ ID NO: 1.

10. The engineered phosphoketolase of any one of claims 1 to 4, wherein the engineered phosphoketolase comprises one or more amino acid substitutions at a residue corresponding to position 63, 221, 323, 442, 499, 504, and/or 548 in SEQ ID NO: 1.

11. The engineered phosphoketolase of any one of claims 1 to 4, wherein the engineered phosphoketolase comprises one or more amino acid substitutions at a residue corresponding to position 63, 64, 65, 156, 158, 219, 220, 221, 436, 438, 440, 442, 478, 504, 505, 542, 547, 548, and/or 549 in SEQ ID NO: 1.

12. The engineered phosphoketolase of any one of claims 1 to 4, wherein the engineered phosphoketolase comprises one or more amino acid substitutions at a residue corresponding to position 43, 69, 73, 113, 145, 167, 183, 230, 248, 266, 276, 306, 336, 337, 349, 379, 420, 449, 451, 460, 513, 518, 560, 562, 581, 585, 606, 614, 620, 634, 700, 773, 779, 781, 795, 800, 802, and/or 813 in SEQ ID NO: 2.

13. The engineered phosphoketolase of any one of claims 1 to 4, wherein the engineered phosphoketolase comprises one or more amino acid substitutions at a residue corresponding to position 43, 349, 420, and/or 795 in SEQ ID NO: 2.

14. The engineered phosphoketolase of any one of claims 1 to 4, wherein the engineered phosphoketolase comprises one or more amino acid substitutions at a residue corresponding to position 43, 69, 73, 113, 145, 167, 183, 230, 276, 306, 336, 349, 379, 420, 449, 451, 460, 513, 560, 562, 585, 606, 614, 620, 634, 700, 779, 781, 795, 800, and/or 802 in SEQ ID NO: 2.

15. The engineered phosphoketolase of any one of claims 1 to 4, wherein the engineered phosphoketolase comprises one or more amino acid substitutions at a residue corresponding to position 69, 73, 113, 145, 167, 183, 230, 276, 349, 379, 420, 449, 460, 513, 562, 585, 606, 634, 700, 779, 795, 800, and/or 802 in SEQ ID NO: 2.

16. The engineered phosphoketolase of any one of claims 1 to 4, wherein the engineered phosphoketolase comprises one or more amino acid substitutions at a residue corresponding to position 73, 167, 230, 336, 449, 451, 513, 560, 562, and/or 620 in SEQ ID NO: 2.

17. The engineered phosphoketolase of any one of claims 1 to 16, wherein the one or more amino acid alterations are conservative amino acid substitutions.

18. The engineered phosphoketolase of any one of claims 1 to 16, wherein the one or more amino acid alterations are non-conservative amino acid substitutions.

19. The engineered phosphoketolase of any one of claims 1 to 4, wherein the one or more amino acid alterations of the engineered phosphoketolase is an alteration described in Table 1.

20. The engineered phosphoketolase of claim 19, wherein the one or more amino acid alternations result in an engineered phosphoketolase comprising:
a) L at a residue corresponding to position 49 in SEQ ID NO: 1;
b) P at a residue corresponding to position 60 in SEQ ID NO: 1;
c) L at a residue corresponding to position 63 in SEQ ID NO: 1;
d) A or S at a residue corresponding to position 64 in SEQ ID NO: 1;
e) Y at a residue corresponding to position 65 in SEQ ID NO: 1;
f) A at a residue corresponding to position 77 in SEQ ID NO: 1;
g) L or V at a residue corresponding to position 104 in SEQ ID NO: 1;
h) F at a residue corresponding to position 136 in SEQ ID NO: 1;
i) A at a residue corresponding to position 156 in SEQ ID NO: 1;
j) I, V, M, or A at a residue corresponding to position 158 in SEQ ID NO: 1;
k) D at a residue corresponding to position 174 in SEQ ID NO: 1;
l) F at a residue corresponding to position 200 in SEQ ID NO: 1;
m) A at a residue corresponding to position 208 in SEQ ID NO: 1;
n) C, N, D, S, M, or E at a residue corresponding to position 219 in SEQ ID NO:1;
o) M at a residue corresponding to position 220 in SEQ ID NO: 1;
p) Q, C, T, V, R, N, M, H, E, or G at a residue corresponding to position 221 in SEQ ID NO:1;
q) R at a residue corresponding to position 239 in SEQ ID NO: 1;
r) P at a residue corresponding to position 257 in SEQ ID NO: 1;
s) A at a residue corresponding to position 267 in SEQ ID NO: 1;
t) R or K at a residue corresponding to position 276 in SEQ ID NO: 1;
u) G at a residue corresponding to position 305 in SEQ ID NO: 1;
v) I at a residue corresponding to position 323 in SEQ ID NO: 1;
w) L at a residue corresponding to position 335 in SEQ ID NO: 1;
x) L at a residue corresponding to position 364 in SEQ ID NO: 1;
y) A at a residue corresponding to position 365 in SEQ ID NO: 1;
z) M at a residue corresponding to position 372 in SEQ ID NO: 1;
aa) H at a residue corresponding to position 377 in SEQ ID NO: 1;
bb) R at a residue corresponding to position 407 in SEQ ID NO: 1;
cc) L at a residue corresponding to position 420 in SEQ ID NO: 1;
dd) A, V, S, C, or E at a residue corresponding to position 436 in SEQ ID NO: 1;
ee) D or T at a residue corresponding to position 438 in SEQ ID NO: 1;
ff) I or K at a residue corresponding to position 440 in SEQ ID NO: 1;
gg) Q, A, D, T, H, M, V, C, S, or I at a residue corresponding to position 442 in SEQ ID NO:1;
hh) V at a residue corresponding to position 447 in SEQ ID NO: 1;
ii) I or M at a residue corresponding to position 478 in SEQ ID NO: 1;
jj) C at a residue corresponding to position 483 in SEQ ID NO: 1;
kk) F at a residue corresponding to position 499 in SEQ ID NO: 1;
ll) A at a residue corresponding to position 504 in SEQ ID NO: 1;
mm) M at a residue corresponding to position 505 in SEQ ID NO: 1;
nn) A, G, W, or I at a residue corresponding to position 542 in SEQ ID NO: 1;
oo) E or A at a residue corresponding to position 547 in SEQ ID NO: 1;
pp) T, I, E, V, M, N, P, or G at a residue corresponding to position 548 in SEQ ID NO:1;
qq) Y at a residue corresponding to position 549 in SEQ ID NO: 1;
rr) S at a residue corresponding to position 567 in SEQ ID NO: 1;
ss) D or E at a residue corresponding to position 572 in SEQ ID NO: 1;
tt) T, S, or C at a residue corresponding to position 584 in SEQ ID NO: 1;
uu) L at a residue corresponding to position 593 in SEQ ID NO: 1;
vv) Q at a residue corresponding to position 611 in SEQ ID NO: 1;
ww) H at a residue corresponding to position 621 in SEQ ID NO: 1;
xx) G at a residue corresponding to position 627 in SEQ ID NO: 1;
yy) P at a residue corresponding to position 641 in SEQ ID NO: 1;
zz) H at a residue corresponding to position 687 in SEQ ID NO: 1;
aaa) L at a residue corresponding to position 759 in SEQ ID NO: 1;
bbb) R at a residue corresponding to position 765 in SEQ ID NO: 1;
ccc) P at a residue corresponding to position 767 in SEQ ID NO: 1;
ddd) Q, K, or R at a residue corresponding to position 781 in SEQ ID NO: 1;
eee) K or R at a residue corresponding to position 786 in SEQ ID NO: 1;
fff) Y at a residue corresponding to position 788 in SEQ ID NO: 1;
ggg) I at a residue corresponding to position 799 in SEQ ID NO: 1; and/or
hhh) W at a residue corresponding to position 804 in SEQ ID NO: 1.

21. The engineered phosphoketolase of claim 19, wherein the one or more amino acid alternations result in an engineered phosphoketolase comprising:
a) L at a residue corresponding to position 49 in SEQ ID NO: 1;
b) P at a residue corresponding to position 60 in SEQ ID NO: 1;
c) L at a residue corresponding to position 63 in SEQ ID NO: 1;
d) A at a residue corresponding to position 77 in SEQ ID NO: 1;
e) L or V at a residue corresponding to position 104 in SEQ ID NO: 1;
f) F at a residue corresponding to position 136 in SEQ ID NO: 1;
g) D at a residue corresponding to position 174 in SEQ ID NO: 1;
h) F at a residue corresponding to position 200 in SEQ ID NO: 1;
i) A at a residue corresponding to position 208 in SEQ ID NO: 1;
j) Q, C, N, or T at a residue corresponding to position 221 in SEQ ID NO: 1;
k) R at a residue corresponding to position 239 in SEQ ID NO: 1;
l) P at a residue corresponding to position 257 in SEQ ID NO: 1;
m) A at a residue corresponding to position 267 in SEQ ID NO: 1;
n) R at a residue corresponding to position 276 in SEQ ID NO: 1;
o) G at a residue corresponding to position 305 in SEQ ID NO: 1;
p) I at a residue corresponding to position 323 in SEQ ID NO: 1;
q) L at a residue corresponding to position 364 in SEQ ID NO: 1;
r) A at a residue corresponding to position 365 in SEQ ID NO: 1;
s) M at a residue corresponding to position 372 in SEQ ID NO: 1;
t) H at a residue corresponding to position 377 in SEQ ID NO: 1;
u) L at a residue corresponding to position 420 in SEQ ID NO: 1;
v) Q at a residue corresponding to position 442 in SEQ ID NO: 1;
w) V at a residue corresponding to position 447 in SEQ ID NO: 1;
x) F at a residue corresponding to position 499 in SEQ ID NO: 1;
y) C at a residue corresponding to position 483 in SEQ ID NO: 1;
z) A at a residue corresponding to position 504 in SEQ ID NO: 1;
aa) T at a residue corresponding to position 548 in SEQ ID NO: 1;
bb) S at a residue corresponding to position 567 in SEQ ID NO: 1;
cc) D or E at a residue corresponding to position 572 in SEQ ID NO: 1;
dd) T, S, or C at a residue corresponding to position 584 in SEQ ID NO: 1;
ee) L at a residue corresponding to position 593 in SEQ ID NO: 1;
ff) Q at a residue corresponding to position 611 in SEQ ID NO: 1;
gg) H at a residue corresponding to position 621 in SEQ ID NO: 1;
hh) G at a residue corresponding to position 627 in SEQ ID NO: 1;
ii) P at a residue corresponding to position 641 in SEQ ID NO: 1;
jj) H at a residue corresponding to position 687 in SEQ ID NO: 1;
kk) L at a residue corresponding to position 759 in SEQ ID NO: 1;
ll) R at a residue corresponding to position 765 in SEQ ID NO: 1;
mm) Q or K at a residue corresponding to position 781 in SEQ ID NO: 1;
nn) K or R at a residue corresponding to position 786 in SEQ ID NO: 1;
oo) Y at a residue corresponding to position 788 in SEQ ID NO: 1;
pp) I at a residue corresponding to position 799 in SEQ ID NO: 1; and/or
qq) W at a residue corresponding to position 804 in SEQ ID NO: 1.

22. The engineered phosphoketolase of claim 19, wherein the one or more amino acid alternations result in an engineered phosphoketolase comprising:
a) L at a residue corresponding to position 63 in SEQ ID NO: 1;
b) A or S at a residue corresponding to position 64 in SEQ ID NO: 1;
c) Y at a residue corresponding to position 65 in SEQ ID NO: 1;
d) F at a residue corresponding to position 136 in SEQ ID NO: 1;
e) A at a residue corresponding to position 156 in SEQ ID NO: 1;
f) I, V, M, or A at a residue corresponding to position 158 in SEQ ID NO: 1;
g) C, N, D, S, M, or E at a residue corresponding to position 219 in SEQ ID NO:1;
h) M at a residue corresponding to position 220 in SEQ ID NO: 1;
i) V, R, N, M, H, E, G, C, or T at a residue corresponding to position 221 in SEQ ID NO:1;
j) A at a residue corresponding to position 267 in SEQ ID NO: 1;
k) K, R at a residue corresponding to position 276 in SEQ ID NO: 1;
l) G at a residue corresponding to position 305 in SEQ ID NO: 1;
m) L at a residue corresponding to position 335 in SEQ ID NO: 1;
n) L at a residue corresponding to position 364 in SEQ ID NO: 1;
o) M at a residue corresponding to position 372 in SEQ ID NO: 1;
p) R at a residue corresponding to position 407 in SEQ ID NO: 1;
q) L at a residue corresponding to position 420 in SEQ ID NO: 1;
r) A, V, S, C, or E at a residue corresponding to position 436 in SEQ ID NO: 1;
s) D, T at a residue corresponding to position 438 in SEQ ID NO: 1;
t) I, K at a residue corresponding to position 440 in SEQ ID NO: 1;
u) A, D, T, H, M, V, C, S, or I at a residue corresponding to position 442 in SEQ ID NO:1;
v) I, M at a residue corresponding to position 478 in SEQ ID NO: 1;
w) A at a residue corresponding to position 504 in SEQ ID NO: 1;
x) M at a residue corresponding to position 505 in SEQ ID NO: 1;
y) A, G, W, or I at a residue corresponding to position 542 in SEQ ID NO: 1;
z) E, A at a residue corresponding to position 547 in SEQ ID NO: 1;
aa) T, I, E, V, M, N, P, or G, at a residue corresponding to position 548 in SEQ ID NO:1;
bb) Y at a residue corresponding to position 549 in SEQ ID NO: 1;
cc) E at a residue corresponding to position 572 in SEQ ID NO: 1;
dd) S or T at a residue corresponding to position 584 in SEQ ID NO: 1;
ee) L at a residue corresponding to position 593 in SEQ ID NO: 1;
ff) G at a residue corresponding to position 627 in SEQ ID NO: 1;
gg) P at a residue corresponding to position 641 in SEQ ID NO: 1;
hh) H at a residue corresponding to position 687 in SEQ ID NO: 1;
ii) P at a residue corresponding to position 767 in SEQ ID NO: 1; and/or
jj) R, K, or Q at a residue corresponding to position 781 in SEQ ID NO: 1.

23. The engineered phosphoketolase of claim 19, wherein the one or more amino acid alternations result in an engineered phosphoketolase comprising:
a) L at a residue corresponding to position 63 in SEQ ID NO: 1;
b) F at a residue corresponding to position 136 in SEQ ID NO: 1;
c) C or T at a residue corresponding to position 221 in SEQ ID NO: 1;
d) A at a residue corresponding to position 267 in SEQ ID NO: 1;
e) R at a residue corresponding to position 276 in SEQ ID NO: 1;
f) G at a residue corresponding to position 305 in SEQ ID NO: 1;
g) L at a residue corresponding to position 364 in SEQ ID NO: 1;
h) M at a residue corresponding to position 372 in SEQ ID NO: 1;
i) L at a residue corresponding to position 420 in SEQ ID NO: 1;
j) A at a residue corresponding to position 504 in SEQ ID NO: 1;
k) D or E at a residue corresponding to position 572 in SEQ ID NO: 1;
l) T, S, or C at a residue corresponding to position 584 in SEQ ID NO: 1;
m) L at a residue corresponding to position 593 in SEQ ID NO: 1;
n) G at a residue corresponding to position 627 in SEQ ID NO: 1;
o) P at a residue corresponding to position 641 in SEQ ID NO: 1;
p) H at a residue corresponding to position 687 in SEQ ID NO: 1; and/or
q) Q or K at a residue corresponding to position 781 in SEQ ID NO: 1.

24. The engineered phosphoketolase of claim 19, wherein the one or more amino acid alternations result in an engineered phosphoketolase comprising:
a) P at a residue corresponding to position 60 in SEQ ID NO: 1;
b) A at a residue corresponding to position 64 in SEQ ID NO: 1;
c) V or L at a residue corresponding to position 104 in SEQ ID NO: 1;
d) F at a residue corresponding to position 136 in SEQ ID NO: 1;
e) I at a residue corresponding to position 158 in SEQ ID NO: 1;
f) D at a residue corresponding to position 174 in SEQ ID NO: 1;
g) Q or H at a residue corresponding to position 221 in SEQ ID NO: 1;
h) A at a residue corresponding to position 267 in SEQ ID NO: 1;
i) L at a residue corresponding to position 335 in SEQ ID NO: 1;
j) A at a residue corresponding to position 365 in SEQ ID NO: 1;
k) R at a residue corresponding to position 407 in SEQ ID NO: 1;
l) A at a residue corresponding to position 436 in SEQ ID NO: 1;
m) V at a residue corresponding to position 447 in SEQ ID NO: 1;
n) F at a residue corresponding to position 499 in SEQ ID NO: 1;
o) G, N, T, or I at a residue corresponding to position 548 in SEQ ID NO: 1;
p) D at a residue corresponding to position 572 in SEQ ID NO: 1;
q) L at a residue corresponding to position 593 in SEQ ID NO: 1;
r) H at a residue corresponding to position 621 in SEQ ID NO: 1;
s) H at a residue corresponding to position 687 in SEQ ID NO: 1;
t) R at a residue corresponding to position 765 in SEQ ID NO: 1;
u) P at a residue corresponding to position 767 in SEQ ID NO: 1;
v) R or Q at a residue corresponding to position 781 in SEQ ID NO: 1;
w) K or R at a residue corresponding to position 786 in SEQ ID NO: 1; and/or
x) Y at a residue corresponding to position 788 in SEQ ID NO: 1.

25. The engineered phosphoketolase of claim 19, wherein the one or more amino acid alternations result in an engineered phosphoketolase comprising:
a) L at a residue corresponding to position 63 in SEQ ID NO: 1;
b) C, T, N, or Q at a residue corresponding to position 221 in SEQ ID NO: 1;
c) I at a residue corresponding to position 323 in SEQ ID NO: 1;
d) Q at a residue corresponding to position 442 in SEQ ID NO: 1;
e) F at a residue corresponding to position 499 in SEQ ID NO: 1;
f) A at a residue corresponding to position 504 in SEQ ID NO: 1; and/or
g) T at a residue corresponding to position 548 in SEQ ID NO: 1.

26. The engineered phosphoketolase of claim 19, wherein the one or more amino acid alternations result in an engineered phosphoketolase comprising:
a) L at position 63 in SEQ ID NO: 1;
b) A or S at position 64 in SEQ ID NO: 1;
c) Y at position 65 in SEQ ID NO: 1;
d) Y at position 156 in SEQ ID NO: 1;
e) I, V, M, or A at position 158 in SEQ ID NO: 1;
f) C, N, D, S, M, or E at position 219 in SEQ ID NO: 1;
g) M at position 220 in SEQ ID NO: 1;
h) V, R, N, M, H, E, G, C, or T at position 221 in SEQ ID NO: 1;
i) A, V, S, C, or E at position 436 in SEQ ID NO: 1;
j) D or T at position 438 in SEQ ID NO: 1;
k) I or K at position 440 in SEQ ID NO: 1;
l) A, D, T, H, M, V, C, S, or I at position 442 in SEQ ID NO: 1;
m) I or M at position 478 in SEQ ID NO: 1;
n) A at position 504 in SEQ ID NO: 1;
o) M at position 505 in SEQ ID NO: 1;
p) A, G, W, or I at position 542 in SEQ ID NO: 1;
q) E or A at position 547 in SEQ ID NO: 1;
r) T, I, E, V, M, N, P, or G at position 548 in SEQ ID NO: 1; and/or
s) Y at position 549 in SEQ ID NO: 1.

27. The engineered phosphoketolase of any one of claims 1 to 4, wherein the one or more amino acid alterations of the engineered phosphoketolase is an alteration described in Table 2.

28. The engineered phosphoketolase of claim 27, wherein the one or more amino acid alternations result in an engineered phosphoketolase comprising:
a) D at a residue corresponding to position 43 in SEQ ID NO: 2;
b) P at a residue corresponding to position 69 in SEQ ID NO: 2;
c) A at a residue corresponding to position 73 in SEQ ID NO: 2;
d) V or L at a residue corresponding to position 113 in SEQ ID NO: 2;
e) F at a residue corresponding to position 145 in SEQ ID NO: 2;
f) I at a residue corresponding to position 167 in SEQ ID NO: 2;
g) D at a residue corresponding to position 183 in SEQ ID NO: 2;
h) N, Q or H at a residue corresponding to position 230 in SEQ ID NO: 2;
i) R at a residue corresponding to position 248 in SEQ ID NO: 2;
j) P at a residue corresponding to position 266 in SEQ ID NO: 2;
k) A at a residue corresponding to position 276 in SEQ ID NO: 2;
l) W at a residue corresponding to position 306 in SEQ ID NO: 2;
m) S at a residue corresponding to position 336 in SEQ ID NO: 2;
n) I at a residue corresponding to position 337 in SEQ ID NO: 2;
o) L at a residue corresponding to position 349 in SEQ ID NO: 2;
p) A at a residue corresponding to position 379 in SEQ ID NO: 2;
q) R at a residue corresponding to position 420 in SEQ ID NO: 2;
r) A at a residue corresponding to position 449 in SEQ ID NO: 2;
s) I at a residue corresponding to position 451 in SEQ ID NO: 2;
t) V at a residue corresponding to position 460 in SEQ ID NO: 2;
u) F at a residue corresponding to position 513 in SEQ ID NO: 2;
v) A at a residue corresponding to position 518 in SEQ ID NO: 2;
w) K, L, T, M, Y, Q, G, or E at a residue corresponding to position 560 in SEQ ID NO: 2;
x) H, G, F, W, C, N, T, or I at a residue corresponding to position 562 in SEQ ID NO: 2;
y) S at a residue corresponding to position 581 in SEQ ID NO: 2;
z) D at a residue corresponding to position 585 in SEQ ID NO: 2;
aa) L at a residue corresponding to position 606 in SEQ ID NO: 2;
bb) V at a residue corresponding to position 614 in SEQ ID NO: 2;
cc) K at a residue corresponding to position 620 in SEQ ID NO: 2;
dd) H at a residue corresponding to position 634 in SEQ ID NO: 2;
ee) H at a residue corresponding to position 700 in SEQ ID NO: 2;
ff) L at a residue corresponding to position 773 in SEQ ID NO: 2;
gg) R at a residue corresponding to position 779 in SEQ ID NO: 2;
hh) P at a residue corresponding to position 781 in SEQ ID NO: 2;
ii) R or Q at a residue corresponding to position 795 in SEQ ID NO: 2;
jj) K or R at a residue corresponding to position 800 in SEQ ID NO: 2;
kk) Y at a residue corresponding to position 802 in SEQ ID NO: 2; and/or
ll) I at a residue corresponding to position 813 in SEQ ID NO: 2.

29. The engineered phosphoketolase of claim 27, wherein the one or more amino acid alternations result in an engineered phosphoketolase comprising:
a) D at a residue corresponding to position 43 in SEQ ID NO: 2;
b) L at a residue corresponding to position 349 in SEQ ID NO: 2;
c) R at a residue corresponding to position 420 in SEQ ID NO: 2; and/or
d) R at a residue corresponding to position 795 in SEQ ID ON: 2.

30. The engineered phosphoketolase of claim 27, wherein the one or more amino acid alternations result in an engineered phosphoketolase comprising:
a) D at a residue corresponding to position 43 in SEQ ID NO: 2;
b) P at a residue corresponding to position 69 in SEQ ID NO: 2;
c) A at a residue corresponding to position 73 in SEQ ID NO: 2;
d) V or L at a residue corresponding to position 113 in SEQ ID NO: 2;
e) F at a residue corresponding to position 145 in SEQ ID NO: 2;
f) I at a residue corresponding to position 167 in SEQ ID NO: 2;
g) D at a residue corresponding to position 183 in SEQ ID NO: 2;
h) Q or H at a residue corresponding to position 230 in SEQ ID NO: 2;
i) A at a residue corresponding to position 276 in SEQ ID NO: 2;
j) W at a residue corresponding to position 306 in SEQ ID NO: 2;
k) S at a residue corresponding to position 336 in SEQ ID NO: 2;
l) L at a residue corresponding to position 349 in SEQ ID NO: 2;
m) A at a residue corresponding to position 379 in SEQ ID NO: 2;
n) R at a residue corresponding to position 420 in SEQ ID NO: 2;
o) A at a residue corresponding to position 449 in SEQ ID NO: 2;
p) I at a residue corresponding to position 451 in SEQ ID NO: 2;
q) V at a residue corresponding to position 460 in SEQ ID NO: 2;
r) F at a residue corresponding to position 513 in SEQ ID NO: 2;
s) K, L, T, M, Y, Q, G, or E at a residue corresponding to position 560 in SEQ ID NO: 2;
t) H, G, F, W, C, N, T, or I at a residue corresponding to position 562 in SEQ ID NO: 2;
u) D at a residue corresponding to position 585 in SEQ ID NO: 2;
v) L at a residue corresponding to position 606 in SEQ ID NO: 2;
w) V at a residue corresponding to position 614 in SEQ ID NO: 2;
x) K at a residue corresponding to position 620 in SEQ ID NO: 2;
y) H at a residue corresponding to position 634 in SEQ ID NO: 2;
z) H at a residue corresponding to position 700 in SEQ ID NO: 2;
aa) R at a residue corresponding to position 779 in SEQ ID NO: 2;
bb) P at a residue corresponding to position 781 in SEQ ID NO: 2;
cc) R or Q at a residue corresponding to position 795 in SEQ ID NO: 2;
dd) K or R at a residue corresponding to position 800 in SEQ ID NO: 2; and/or
ee) Y at a residue corresponding to position 802 in SEQ ID NO: 2.

31. The engineered phosphoketolase of claim 27, wherein the one or more amino acid alternations result in an engineered phosphoketolase comprising:
a) P at a residue corresponding to position 69 in SEQ ID NO: 2;
b) A at a residue corresponding to position 73 in SEQ ID NO: 2;
c) L or V at a residue corresponding to position 113 in SEQ ID NO: 2;
d) F at a residue corresponding to position 145 in SEQ ID NO: 2;
e) I at a residue corresponding to position 167 in SEQ ID NO: 2;
f) D at a residue corresponding to position 183 in SEQ ID NO: 2;
g) Q or H at a residue corresponding to position 230 in SEQ ID NO: 2;
h) A at a residue corresponding to position 276 in SEQ ID NO: 2;
i) L at a residue corresponding to position 349 in SEQ ID NO: 2;
j) A at a residue corresponding to position 379 in SEQ ID NO: 2;
k) R at a residue corresponding to position 420 in SEQ ID NO: 2;
l) A at a residue corresponding to position 449 in SEQ ID NO: 2;
m) V at a residue corresponding to position 460 in SEQ ID NO: 2;
n) F at a residue corresponding to position 513 in SEQ ID NO: 2;
o) T, N, I, or G at a residue corresponding to position 562 in SEQ ID NO: 2;
p) D at a residue corresponding to position 585 in SEQ ID NO: 2;
q) L at a residue corresponding to position 606 in SEQ ID NO: 2;
r) H at a residue corresponding to position 634 in SEQ ID NO: 2;
s) H at a residue corresponding to position 700 in SEQ ID NO: 2;
t) R at a residue corresponding to position 779 in SEQ ID NO: 2;
u) Q or R at a residue corresponding to position 795 in SEQ ID NO: 2;
v) K or R at a residue corresponding to position 800 in SEQ ID NO: 2; and/or
w) Y at a residue corresponding to position 802 in SEQ ID NO: 2.

32. The engineered phosphoketolase of claim 27, wherein the one or more amino acid alternations result in an engineered phosphoketolase comprising:
a) A at a residue corresponding to position 73 in SEQ ID NO: 2;
b) I at a residue corresponding to position 167 in SEQ ID NO: 2;
c) Q or H at a residue corresponding to position 230 in SEQ ID NO: 2;
d) S at a residue corresponding to position 336 in SEQ ID NO: 2;
e) A at a residue corresponding to position 449 in SEQ ID NO: 2;
f) I at a residue corresponding to position 451 in SEQ ID NO: 2;
g) F at a residue corresponding to position 513 in SEQ ID NO: 2;
h) K, L, T, M, Y, Q, G, or E at a residue corresponding to position 560 in SEQ ID NO: 2;
i) H, G, F, W, C, N, T, or I at a residue corresponding to position 562 in SEQ ID NO: 2; and/or
j) K at a residue corresponding to position 620 in SEQ ID NO: 2.

33. The engineered phosphoketolase of any one of claims 1 to 32, wherein the one or more amino acid alterations comprises at least two, three, four, five, six, seven, eight, nine or ten amino acid alterations.

34. A recombinant nucleic acid encoding the engineered phosphoketolase of any one of claims 1 to 33.

35. The recombinant nucleic acid of claim 34, wherein the nucleic acid comprises a nucleotide sequence encoding the engineered phosphoketolase is operatively linked to a promoter.

36. A vector comprising the nucleic acid of claim 35.

37. A non-naturally occurring microbial organism comprising a recombinant nucleic acid encoding an engineered phosphoketolase selected from any one of claims 1 to 33.

38. The non-naturally occurring microbial organism of claim 37, further comprising an exogenous nucleic acid encoding:
(a) an acetate kinase and an acetyl-CoA transferase, synthetase, or ligase, wherein the acetate kinase catalyzes the conversion of acetyl-phosphate to acetate and the acetyl-CoA transferase, synthetase, or ligase catalyzes the conversion of acetate to acetyl-CoA;
(b) a phosphotransacetylase, wherein the phosphotransacetylase catalyzes the conversion of acetyl-phosphate to acetyl-CoA;
(c) a pyruvate dehydrogenase, a pyruvate ferredoxin oxidoreductase, or a pyruvate:NADP+ oxidoreductase, wherein the pyruvate dehydrogenase, the pyruvate ferredoxin oxidoreductase, or the pyruvate:NADP+ oxidoreductase catalyzes the conversion of pyruvate to acetyl-CoA and carbon dioxide; or
(d) a pyruvate formate lyase, wherein the pyruvate formate lyase catalyzes the conversion of pyruvate to acetyl-CoA and formate.

39. The non-naturally occurring microbial organism of claim 37 or 38, further comprising exogenous nucleic acids encoding a combination of enzymes that catalyze the conversion of glyceraldehyde-3-phosphate to pyruvate, wherein the combination of enzymes comprise a glyceraldehyde-3-phosphate dehydrogenase, a phosphoglycerate kinase, a phosphoglyceromutase, an enolase, a pyruvate kinase and a PTS-dependent substrate import.

40. The non-naturally occurring microbial organism of any one of claims 37 to 39, wherein the exogenous nucleic acid is heterologous.

41. The non-naturally occurring microbial organism of any one of claims 37 to 39, wherein the exogenous nucleic acid is homologous.

42. The non-naturally occurring microbial organism of any one of claims 37 to 41, wherein said non-naturally occurring microbial organism further comprises a pathway capable of producing a bioderived compound from acetyl-CoA.

43. The non-naturally occurring microbial organism of claim 42, wherein said bioderived compound is an alcohol, a glycol, an organic acid, an alkene, a diene, an organic amine, an organic aldehyde, a vitamin, a nutraceutical or a pharmaceutical.

44. The non-naturally occurring microbial organism of claim 43, wherein said alcohol is selected from the group consisting of:
(a) a biofuel alcohol, wherein said biofuel is a primary alcohol, a secondary alcohol, a diol or triol comprising C3 to C10 carbon atoms;
(b) n-propanol or isopropanol; and
(c) a fatty alcohol, wherein said fatty alcohol comprises C4 to C27 carbon atoms, C8 to C18 carbon atoms, C12 to C18 carbon atoms, or C12 to C14 carbon atoms.

45. The non-naturally occurring microbial organism of claim 44, wherein said biofuel alcohol is 1-propanol, isopropanol, 1-butanol, isobutanol, 1-pentanol, isopentenol, 2-methyl-1-butanol, 3-methyl-1-butanol, 1-hexanol, 3-methyl-1-pentanol, 1-heptanol, 4-methyl-1-hexanol, and 5-methyl-1-hexanol.

46. The non-naturally occurring microbial organism of claim 44, wherein said diol is a propanediol or a butanediol.

47. The non-naturally occurring microbial organism of claim 46, wherein said butanediol is 1,4 butanediol, 1,3-butanediol or 2,3-butanediol.

48. The non-naturally occurring microbial organism of claim 42, wherein said bioderived compounds is selected from the group consisting of:
(a) 1,4-butanediol or an intermediate thereto, wherein said intermediate is optionally 4-hydroxybutanoic acid (4-HB);
(b) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol;
(c) 1,3-butanediol or an intermediate thereto, wherein said intermediate is optionally 3-hydroxybutyrate (3-HB), 2,4-pentadienoate, crotyl alcohol or 3-buten-1-ol;
(d) adipate, 6-aminocaproic acid, caprolactam, hexamethylenediamine, levulinic acid or an intermediate thereto, wherein said intermediate is optionally adipyl-CoA or 4-aminobutyryl-CoA;
(e) methacrylic acid or an ester thereof, 3-hydroxyisobutyrate, 2-hydroxyisobutyrate, or an intermediate thereto, wherein said ester is optionally methyl methacrylate or poly(methyl methacrylate);
(f) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto;
(g) succinic acid or an intermediate thereto; and
(h) a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

49. The non-naturally occurring microbial organism of any one of claims 37 to 48, wherein the non-naturally occurring microbial organism is in a substantially anaerobic culture medium.

50. The non-naturally occurring microbial organism of any one of claims 37 to 49, wherein the microbial organism is a species of bacteria, yeast, or fungus.

51. The non-naturally occurring microbial organism of any one of claims 37 to 50, wherein the non-naturally occurring microbial organism is capable of producing at least 10% more acetyl-phosphate, acetyl-CoA or a bioderived compound compared to a control microbial organism that does not comprise the nucleic acid of claim 34 or 35.

52. A method for producing a bioderived compound, comprising culturing the non-naturally occurring microbial organism of any one of claims 42 to 48 under conditions and for a sufficient period of time to produce the bioderived compound.

53. The method of claim 52, wherein said method further comprises separating the bioderived from other components in the culture.

54. The method of claim 53, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

55. A culture medium comprising said bioderived compound produced by the method of any one of claims 52 to 54, wherein said bioderived compound has a carbon-12, carbon-13 and carbon-14 isotope ratio that reflects an atmospheric carbon dioxide uptake source.

56. A bioderived compound produced according to the method of any one of claims 52 to 54.

57. The bioderived compound of claim 56, wherein said bioderived compound has an Fm value of at least 80%, at least 85%, at least 90%, at least 95% or at least 98%.

58. A composition comprising said bioderived compound of claim 56 or 57 and a compound other than said bioderived compound.

59. The composition of claim 58, wherein said compound other than said bioderived compound is a trace amount of a cellular portion of a non-naturally occurring microbial organism having a bioderived compound pathway.

60. A composition comprising the bioderived compound of claim 56 or 57, or a cell lysate or culture supernatant thereof.


SEQUENCE LISTING (5 sequences)

SEQ ID NO: 1 - protein, 826 aa, Bifidobacterium adolescentis
MMTSPVIGTPWKKLNAPVSEEAIEGVDKYWRAANYLSIGQIYLRSNPLMKEPFTREDVKHRLVGHWGTTPGLNFLIGHINRLIADHQQNTVIIMGPGHGGPAGTAQSYLDGTYTEYFPNITKDEAGLQKFFRQFSYPGGIPSHYAPETPGSIHEGGELGYALSHAYGAVMNNPSLFVPAIVGDGEAETGPLATGWQSNKLINPRTDGIVLPILHLNGYKIANPTILSRISDEELHEFFHGMGYEPYEFVAGFDNEDHLSIHRRFAELFETVFDEICDIKAAAQTDDMTRPFYPMIIFRTPKGWTCPKFIDGKKTEGSWRSHQVPLASARDTEAHFEVLKNWLESYKPEELFDENGAVKPEVTAFMPTGELRIGENPNANGGRIREELKLPKLEDYEVKEVAEYGHGWGQLEATRRLGVYTRDIIKNNPDSFRIFGPDETASNRLQAAYDVTNKQWDAGYLSAQVDEHMAVTGQVTEQLSEHQMEGFLEGYLLTGRHGIWSSYESFVHVIDSMLNQHAKWLEATVREIPWRKPISSMNLLVSSHVWRQDHNGFSHQDPGVTSVLLNKCFNNDHVIGIYFPVDSNMLLAVAEKCYKSTNKINAIIAGKQPAATWLTLDEARAELEKGAAEWKWASNVKSNDEAQIVLAATGDVPTQEIMAAADKLDAMGIKFKVVNVVDLVKLQSAKENNEALSDEEFAELFTEDKPVLFAYHSYARDVRGLIYDRPNHDNFNVHGYEEQGSTTTPYDMVRVNNIDRYELQAEALRMIDADKYADKINELEAFRQEAFQFAVDNGYDHPDYTDWVYSGVNTNKQGAISATAATAGDNE

SEQ ID NO: 2 - protein, 842 aa, Collinsella aerofaciens
MAKTLGTPWQKLGHEVPASELEGVDLYWRASNYLSVGQIYLRSNPLMRADFVDDKTGEARDFGRPDVKHRLVGHWGTTPGINFLFGHVNRLIADHNQNAIFLMGPGHGGPAGTAQSLLDGTYREIRPDITNDEAGLQKFFRQFSYPGGIPSHFAPETPGSIHEGGELGYTLSHAYGAVMDNPSLLAVAVVGDGESETGPLATSWQSNKLVNPATDGIVLPILHLNGYKIANPTILARVSDEELTKFFEGMGYKPHFFIAGFDDESHASIHERFAALFEQVFDEICDIKATAQAQAAAGETVVRPAYPMIVFRTPKGWTCPKQIDGKKTEDSWRAHQVPLASAKDTHEHFRVLREWLRSYKPEELFTPEGQVRPEVTAFMPTGELRIGANPNANGGKVRRELELPDIHAHEIPVAEKGHGWGSTEAARVFGEYTADVLAKNMDDFRIFGPDETASNRLQAAYRVTKKQWDAGFYEDEANDELLAGSGKVVEQLSEHQCEGFLEAYVLTGRSGVWSSYESFVHVVDSMVNQHCKWLEATKREIPWRAPISGLNILLSSHVWRQDHNGFSHQDPGFIDLLLNKANDTHIVNAYYPADANMALAVAERVYQSTDCVNAIFCGKQPAPTFQTVDEAKAELAEGVANWEWASTADSLGEADVVVATCGDVPTLEALAATDMLRELGIKVWFVNVVDLLKIQNVCENDQAISDERWAELFGCGEKPVLFAFHAYAGTIRRLIWNRPGHDAFRVHGYEEKGSTTTPFDMLRLNNMDRWALAADVLRMVDAVKFAEQIDEWEAFRTEAFEFACDEGFDHPAFTDWVWPDAAAATAADGALSATQLTAGDNE

SEQ ID NO: 3 - DNA, 2478 bp, Bifidobacterium adolescentis
atgatgacgagcccagttattgggaccccttggaaaaagttaaacgccccggtaagtgaggaagctatagaaggagtggataaatactggagggcggcaaattatttgtcgatcggccaaatttatctccgatctaacccgcttatgaaagagccctttacacgtgaagacgtcaaacatcgcctggtgggtcactggggcactaccccgggtctgaatttcctgatcggccatattaatcggctaatcgcggatcaccagcagaacacggttattatcatggggccgggtcatggcgggccagcaggtaccgcccaatcatacctggatggaacgtataccgaatactttcccaatataactaaagacgaagcgggcttgcagaagttctttcgccagttcagctatccgggtggcattccgtcccactatgcccctgaaactccaggatctatccatgagggcggtgaactgggatacgcattgtcccacgcttatggcgcggtaatgaacaacccaagcctgtttgtgcctgcgattgtcggtgatggtgaagccgaaaccggcccgctcgctacaggctggcaaagtaacaaactgattaatccgcgtaccgatggtatcgttctcccgattctgcatttaaacggctataaaatcgcgaacccaacgattttatcgcgcatctcagacgaggagctgcatgagttttttcatgggatgggctacgaaccctatgaatttgtggctgggttcgataatgaggatcacttaagtatacaccgtcgtttcgcagaacttttcgaaaccgtgtttgacgagatttgcgacattaaagccgccgcgcagactgacgatatgacgcgcccgttttatccgatgatcatcttccgaaccccgaagggctggacatgtccgaaatttatcgatggtaaaaagactgagggttcttggcgctcgcaccaagttcctctggcaagcgcccgtgataccgaagcgcatttcgaagtcctgaaaaattggcttgaaagctacaaacctgaggaattgtttgacgaaaatggcgcagtcaaacctgaagtgacggcattcatgccaactggcgagttacgaattggtgaaaacccgaatgccaatgggggccgcattagggaagagctaaaattaccgaagctggaagattacgaggtaaaggaagtcgcggaatatggacatggttgggggcagctggaggcgacccgtcgcctgggagtgtacacacgtgatatcattaaaaacaacccagacagctttcgcatctttggcccagatgaaaccgcttcaaaccgtttacaggccgcatatgatgttaccaacaaacaatgggacgctggctatttgagtgctcaggtggatgaacacatggcggtgacgggtcaggtgaccgaacagctgagtgaacatcaaatggaaggctttcttgagggttatctgttgaccggtcggcacggtatctggtcctcgtatgagtctttcgtacacgtcattgatagcatgctcaatcagcatgccaagtggctggaagctactgttcgggaaatcccgtggcgcaaaccgatttcatctatgaacctgctggttagtagccatgtttggaggcaggaccataatggattctcgcaccaagatcccggtgtaacgtccgtacttctgaacaaatgctttaacaatgaccatgtaataggaatttacttccccgtggatagcaacatgctcttggcggttgcggaaaaatgttacaaatccactaacaaaatcaatgctatcatcgcaggaaagcagccagccgcgacatggctgacgttagatgaagctagagcggaactggagaaaggcgcagcagaatggaaatgggcctccaatgttaagtctaacgatgaagctcagattgtgcttgcggccaccggtgacgtcccgacccaagaaattatggccgcagccgataagttagacgccatgggcatcaaatttaaagtcgtaaatgtggttgatctggtgaaactccaatctgcgaaggaaaataacgaggctttgtcagacgaagagtttgcggaactgttcaccgaagataaaccggtcctgttcgcctaccattcgtacgcgcgcgatgttcgtgggctgatttatgatcgtccaaaccatgacaattttaatgtccacgggtatgaagaacagggtagtacgacaacgccgtacgatatggtgcgggtaaataatatagatcgttatgagctgcaagcagaggcgctacgcatgatcgacgcggataaatacgccgacaaaattaatgaattagaagcttttcgccaggaagctttccagtttgcagtcgataacggctatgatcatccagactacactgactgggtgtatagcggggtgaatactaacaaacagggtgcgattagtgcaaccgccgccaccgccggcgacaatgaa

SEQ ID NO: 4 - DNA, 2526 bp, Collinsella aerofaciens
atggcaaaaaccttagggactccgtggcagaagttgggtcacgaagtacccgcgagcgagcttgaaggcgtcgatctgtactggcgtgcctccaactatctgtcagttggacaaatatatctgaggtctaatccactgatgcgcgctgactttgtggatgataaaacaggcgaagcccgcgacttcggtcgtccggatgtgaaacatcggctcgtcggccattggggtacgacccctggcatcaactttctgttcgggcacgtgaatcgattgattgcggaccataaccagaacgcaatctttctaatgggaccgggccacggtggcccagcgggtacggcacagtcgctcctggacgggacctaccgcgagattagaccggatattactaatgatgaagccggccttcaaaaattctttcgtcagtttagctatccgggtggtatcccgagtcatttcgctccagagacacctggatcgattcatgaagggggagaactaggctacaccttaagccacgcgtatggcgcggttatggacaatccgtctttgctggcggtcgccgtagtgggtgatggtgagagtgaaacgggacctcttgcgacctcctggcagagcaacaaactggttaatccggccacggatggcattgtgctgcccatccttcatctgaacggctacaagattgctaacccgaccatcctcgcccgtgtgtccgatgaagaactgactaaatttttcgagggcatgggttataaaccccatttctttattgcagggttcgatgacgaatcacacgcttcgatccacgagcgctttgcggcgctattcgaacaagtctttgacgagatttgcgatataaaggccacggcacaggcccaagcggcggcaggggaaacagtagtacgccctgcatatccgatgattgtttttcgtacaccaaaaggctggacctgtccgaaacagatcgacgggaaaaaaaccgaagattcttggcgcgcacaccaggttccgctggcttccgccaaagatactcatgagcatttccgcgtgctgcgtgaatggttacggagctacaaacccgaagaattgtttacccccgagggtcaagtgcgtccggaggtcaccgctttcatgcctacgggtgaactgcgtataggtgccaatccgaacgcaaatggcggcaaagtgcgccgagaattagaactcccggatatacacgcgcatgaaatcccagttgcggaaaagggccatgggtggggctcgaccgaggctgctcgggtgtttggagaatataccgctgatgtactggccaagaacatggacgattttcgcatttttggtccagatgaaacagccagtaaccgactgcaagcggcttatagggtcacgaaaaaacagtgggatgcgggtttctacgaagatgaggcaaatgacgagttgttagccgggagcgggaaggtggtggagcagttatcagaacatcaatgcgaaggctttttagaagcatacgtcctgacgggtcgcagtggtgtttggagcagttacgaaagcttcgtccacgtcgtggactccatggttaaccagcactgtaagtggctggaagcgaccaaacgagaaattccgtggagagcccctatcagcggcttgaatattcttctgtcgagtcacgtctggcgccaggaccataacggattttcgcaccaagatccaggctttatcgatcttttattaaataaggcaaatgatacccatatcgtaaacgcctattacccggcggacgcgaacatggctctggccgtggcggagcgtgtctatcagtctacggactgcgttaatgcgattttttgcggaaaacagcccgcaccgactttccaaacggtggatgaagccaaagccgaactggccgagggcgtagcgaactgggaatgggcatcaactgcggactcactgggggaagcagacgtcgtggttgcgacctgtggggatgtacctaccctggaagccctcgcggctactgacatgctacgtgaactgggcatcaaagtctggttcgtaaatgttgttgacctgttgaaaatccagaatgtctgtgaaaatgatcaagcgatttcagatgagcggtgggccgagctgtttgggtgcggcgaaaaaccggtgcttttcgcgtttcatgcgtatgcgggtacaattcgtcggctgatctggaaccgtccgggacatgatgcgtttagagtacacggttatgaggaaaaaggttctaccacaaccccgtttgatatgctacgcttgaacaacatggatcgctgggccctcgcagccgatgtgctgcgcatggttgatgctgttaaattcgcggagcaaattgacgaatgggaagcattcagaacggaagcattcgaatttgcctgcgatgaaggctttgatcacccggccttcactgattgggtgtggcccgacgccgccgcggcaaccgctgccgacggtgcgctgagcgcgacacagctcacggctggcgataatgaa

SEQ ID NO: 5 - protein, 846 aa, Artificial Sequence
  Description of Artificial Sequence Synthetic polypeptide
  MOD_RES (3)..(6): Any amino acid
  MOD_RES (7)..(7): Leucine or Isoleucine
  MOD_RES (12)..(12): Any amino acid
  MOD_RES (15)..(17): Any amino acid
  MOD_RES (19)..(22): Any amino acid
  MOD_RES (23)..(23): Leucine or Isoleucine
  MOD_RES (28)..(28): Any amino acid
  MOD_RES (33)..(33): Any amino acid
  MOD_RES (38)..(38): Any amino acid
  MOD_RES (50)..(52): Any amino acid
  MOD_RES (54)..(65): Any amino acid
  MOD_RES (67)..(67): Any amino acid
  MOD_RES (83)..(83): Leucine or Isoleucine
  MOD_RES (87)..(87): Any amino acid
  MOD_RES (90)..(90): Any amino acid
  MOD_RES (98)..(98): Any amino acid
  MOD_RES (101)..(103): Any amino acid
  MOD_RES (104)..(104): Leucine or Isoleucine
  MOD_RES (119)..(119): Any amino acid
  MOD_RES (125)..(125): Any amino acid
  MOD_RES (127)..(128): Any amino acid
  MOD_RES (133)..(133): Any amino acid
  MOD_RES (155)..(155): Any amino acid
  MOD_RES (172)..(172): Any amino acid
  MOD_RES (187)..(189): Any amino acid
  MOD_RES (191)..(191): Any amino acid
  MOD_RES (197)..(197): Any amino acid
  MOD_RES (205)..(205): Any amino acid
  MOD_RES (212)..(212): Any amino acid
  MOD_RES (215)..(215): Any amino acid
  MOD_RES (238)..(238): Any amino acid
  MOD_RES (240)..(240): Any amino acid
  MOD_RES (246)..(247): Any amino acid
  MOD_RES (250)..(250): Any amino acid
  MOD_RES (255)..(255): Any amino acid
  MOD_RES (257)..(258): Any amino acid
  MOD_RES (260)..(260): Any amino acid
  MOD_RES (267)..(267): Any amino acid
  MOD_RES (269)..(269): Any amino acid
  MOD_RES (273)..(273): Any amino acid
  MOD_RES (277)..(277): Any amino acid
  MOD_RES (281)..(281): Any amino acid
  MOD_RES (292)..(292): Any amino acid
  MOD_RES (295)..(304): Any amino acid
  MOD_RES (307)..(307): Any amino acid
  MOD_RES (312)..(312): Any amino acid
  MOD_RES (324)..(324): Any amino acid
  MOD_RES (332)..(332): Any amino acid
  MOD_RES (336)..(336): Any amino acid
  MOD_RES (345)..(345): Any amino acid
  MOD_RES (348)..(349): Any amino acid
  MOD_RES (352)..(352): Any amino acid
  MOD_RES (355)..(356): Any amino acid
  MOD_RES (359)..(359): Any amino acid
  MOD_RES (368)..(370): Any amino acid
  MOD_RES (372)..(372): Any amino acid
  MOD_RES (374)..(374): Any amino acid
  MOD_RES (390)..(390): Any amino acid
  MOD_RES (398)..(399): Any amino acid
  MOD_RES (401)..(401): Any amino acid
  MOD_RES (404)..(404): Any amino acid
  MOD_RES (407)..(407): Any amino acid
  MOD_RES (408)..(408): Leucine or Isoleucine
  MOD_RES (409)..(411): Any amino acid
  MOD_RES (413)..(415): Any amino acid
  MOD_RES (419)..(419): Any amino acid
  MOD_RES (425)..(426): Any amino acid
  MOD_RES (429)..(429): Any amino acid
  MOD_RES (431)..(432): Any amino acid
  MOD_RES (434)..(434): Any amino acid
  MOD_RES (437)..(437): Any amino acid
  MOD_RES (439)..(439): Any amino acid
  MOD_RES (440)..(440): Leucine or Isoleucine
  MOD_RES (441)..(442): Any amino acid
  MOD_RES (444)..(444): Any amino acid
  MOD_RES (446)..(446): Any amino acid
  MOD_RES (465)..(465): Any amino acid
  MOD_RES (468)..(468): Any amino acid
  MOD_RES (475)..(478): Any amino acid
  MOD_RES (480)..(481): Any amino acid
  MOD_RES (484)..(485): Any amino acid
  MOD_RES (487)..(488): Any amino acid
  MOD_RES (490)..(490): Any amino acid
  MOD_RES (492)..(492): Any amino acid
  MOD_RES (500)..(500): Any amino acid
  MOD_RES (506)..(506): Any amino acid
  MOD_RES (508)..(508): Any amino acid
  MOD_RES (513)..(513): Any amino acid
  MOD_RES (515)..(515): Any amino acid
  MOD_RES (526)..(526): Any amino acid
  MOD_RES (530)..(530): Any amino acid
  MOD_RES (534)..(534): Any amino acid
  MOD_RES (541)..(541): Any amino acid
  MOD_RES (548)..(548): Any amino acid
  MOD_RES (552)..(553): Any amino acid
  MOD_RES (555)..(555): Leucine or Isoleucine
  MOD_RES (557)..(557): Any amino acid
  MOD_RES (576)..(579): Any amino acid
  MOD_RES (584)..(585): Any amino acid
  MOD_RES (588)..(588): Any amino acid
  MOD_RES (590)..(593): Any amino acid
  MOD_RES (595)..(595): Any amino acid
  MOD_RES (597)..(597): Any amino acid
  MOD_RES (599)..(599): Any amino acid
  MOD_RES (602)..(602): Any amino acid
  MOD_RES (608)..(609): Any amino acid
  MOD_RES (611)..(611): Any amino acid
  MOD_RES (615)..(616): Any amino acid
  MOD_RES (620)..(621): Any amino acid
  MOD_RES (627)..(627): Any amino acid
  MOD_RES (629)..(630): Any amino acid
  MOD_RES (632)..(632): Any amino acid
  MOD_RES (636)..(636): Any amino acid
  MOD_RES (640)..(641): Any amino acid
  MOD_RES (643)..(643): Any amino acid
  MOD_RES (645)..(645): Any amino acid
  MOD_RES (647)..(647): Any amino acid
  MOD_RES (651)..(653): Any amino acid
  MOD_RES (655)..(656): Any amino acid
  MOD_RES (659)..(660): Any amino acid
  MOD_RES (662)..(662): Any amino acid
  MOD_RES (664)..(665): Any amino acid
  MOD_RES (671)..(671): Any amino acid
  MOD_RES (673)..(674): Any amino acid
  MOD_RES (677)..(677): Any amino acid
  MOD_RES (679)..(679): Any amino acid
  MOD_RES (681)..(683): Any amino acid
  MOD_RES (687)..(689): Any amino acid
  MOD_RES (696)..(696): Any amino acid
  MOD_RES (698)..(698): Leucine or Isoleucine
  MOD_RES (700)..(702): Any amino acid
  MOD_RES (708)..(708): Leucine or Isoleucine
  MOD_RES (712)..(713): Any amino acid
  MOD_RES (718)..(721): Any amino acid
  MOD_RES (728)..(728): Any amino acid
  MOD_RES (730)..(730): Any amino acid
  MOD_RES (733)..(735): Any amino acid
  MOD_RES (737)..(737): Any amino acid
  MOD_RES (740)..(740): Any amino acid
  MOD_RES (744)..(744): Any amino acid
  MOD_RES (747)..(747): Any amino acid
  MOD_RES (749)..(749): Any amino acid
  MOD_RES (756)..(756): Any amino acid
  MOD_RES (763)..(763): Any amino acid
  MOD_RES (766)..(766): Any amino acid
  MOD_RES (768)..(768): Any amino acid
  MOD_RES (771)..(771): Any amino acid
  MOD_RES (774)..(775): Any amino acid
  MOD_RES (777)..(777): Any amino acid
  MOD_RES (779)..(780): Any amino acid
  MOD_RES (784)..(784): Any amino acid
  MOD_RES (787)..(787): Any amino acid
  MOD_RES (789)..(789): Any amino acid
  MOD_RES (791)..(792): Any amino acid
  MOD_RES (796)..(796): Any amino acid
  MOD_RES (801)..(801): Any amino acid
  MOD_RES (808)..(808): Any amino acid
  MOD_RES (810)..(810): Any amino acid
  MOD_RES (812)..(812): Any amino acid
  MOD_RES (816)..(817): Any amino acid
  MOD_RES (822)..(828): Any amino acid
  MOD_RES (830)..(832): Any amino acid
  MOD_RES (835)..(835): Leucine or Isoleucine
  MOD_RES (839)..(840): Any amino acid
MMXXXXXGTPWXKLXXXVXXXXXEGVDXYWRAXNYLSXGQIYLRSNPLMXXXFXXXXXXXXXXXXRXDVKHRLVGHWGTTPGXNFLXGHXNRLIADHXQNXXXXMGPGHGGPAGTAQSXLDGTYXEXXPBITXDEAGLQKFFRQFSYPGGIPSHXAPETPGSIHEGGELGYXLSHAYGAVMBNPSLXXXAXVGDGEXETGPLATXWQSNKLXNPXTDGIVLPILHLNGYKIANPTILXRXSDEELXXFFXGMGYXPXXFXAGFDBEXHXSIHXRFAXLFEXVFDEICDIKAXAQXXXXXXXXXXRPXYPMIXFRTPKGWTCPKXIDGKKTEXSWRXHQVPLASAXDTXXHFXVLXXWLXSYKPEELFXXXGXVXPEVTAFMPTGELRIGXNPNANGGXXRXELXLPXXXXXEXXXVAEXGHGWGXXEAXRXXGXYTXDXXXXNXDXFRIFGPDETASNRLQAAYXVTXKQWDAGXXXXZXXDEXXAXXGXVXEQLSEHQXEGFLEXYXLTGRXGXWSSYESFVHVXDSMXNQHXKWLEATXREIPWRXPISXXNXLXSSHVWRQDHNGFSHQDPGXXXXLLNKXXNBXHXXXXYXPXDXNMXLAVAEXXYXSTBXXNAIXXGKQPAXTXXTXDEAXAELXXGXAXWXWASXXXSXXEAXXVXAXXGDVPTXEXXAAXDXLXXXGIKXXXVNVVDLXKXQXXXENBZAXSDEXXAELFXXXXKPVLFAXHXYAXXXRXLIXBRPXHDXFXVHGYEEXGSTTTPXDMXRXNNXDRXXLXAXXLRMXDAXKXAXXIBEXEAFRXEAFZFAXDXGXDHPXXTDWVXXXXXXXTXXXGAXSATXXTAGDNE